(19) United States
(12) Patent Application Publication
(10) Pub. No.: US 2026/0242833 A1
(43) Pub. Date: Aug. 20, 2026

(54) ENGINEERED TRANS-ENOYL COA REDUCTASE AND METHODS OF MAKING AND USING

(71) Applicant: Genomatica, Inc., San Diego, CA (US)

(72) Inventors: Amit M. Shah, San Diego, CA (US); Harish Nagarajan, San Diego, CA (US); Michael Angus Noble, San Diego, CA (US)

(21) Appl. No.: 19/389,696

(22) Filed: Nov. 14, 2025

Related U.S. Application Data
(63) Continuation of application No. 17/605,120, filed on Apr. 24, 2020, now abandoned.
(60) Provisional application No. 62/837,888, filed on Apr. 24, 2019.
(60) Provisional application No. 62/860,160, filed on Jun. 11, 2019.
(60) Provisional application No. 62/860,123, filed on Jun. 11, 2019.

(51) Int. Cl. C12P 13/00; C12N 1/20; C12N 9/02; C12N 9/10; C12N 15/52; C12N 15/70; C12P 7/18; C12P 7/40; C12P 7/44; C12P 7/62; C12P 17/08; C12P 17/10

(52) U.S. Cl. CPC C12P 13/001; C12N 1/20; C12N 9/0008; C12N 9/001; C12N 9/1096; C12N 15/52; C12N 15/70; C12P 7/18; C12P 7/40; C12P 7/44; C12P 7/62; C12P 13/005; C12P 17/08; C12P 17/10; C12Y 103/01044; C12Y 206/01; C12Y 103/01038

(57) ABSTRACT

Disclosed are trans-enoyl CoA reductase (TER) enzymes and nucleic acids encoding them. In some cases, the TER enzymes are non-natural, engineered trans-enoyl CoA reductase. Also disclosed are biosynthetic methods and engineered microorganisms that enhance or improve the biosynthesis of 6-aminocaproate, hexamethylenediamine, caproic acid, caprolactone, or caprolactam. The engineered microorganisms include exogenous TER and in some cases engineered TER.

### PRIORITY CLAIM

This application is a continuation of U.S. application Ser. No. 17/605,120, filed Oct. 20, 2021, which claims priority to International Application No. PCT/US2020/029788, filed Apr. 24, 2020, which claims the benefit of U.S. Provisional Patent Application Ser. No. 62/837,888, filed Apr. 24, 2019; 62/860,123, filed Jun. 11, 2019; and 62/860,160, filed Jun. 11, 2019, the disclosures of which are incorporated by reference herein in their entirety.

### REFERENCE TO SEQUENCE LISTING

This application contains a Sequence Listing in a computer readable form, submitted via Patent Center. The entire contents of the text file entitled “GN00098US2_Sequence_Listing.txt” created on Jun. 17, 2022, and having a size of 154,446 bytes, is incorporated herein by reference.

### BACKGROUND

Nylons are polyamides that can be synthesized by the condensation polymerization of a diamine with a dicarboxylic acid or the condensation polymerization of lactams. Nylon 6,6 is produced by reaction of hexamethylenediamine (HMD) and adipic acid, while nylon 6 is produced by a ring opening polymerization of caprolactam. Therefore, adipic acid, hexamethylenediamine, and caprolactam are important intermediates in nylon production.

Microorganisms have been engineered to produce some of the nylon intermediates. However, engineered microorganisms can produce undesirable byproducts as a result of undesired enzymatic activity on pathway intermediates and final products. Such byproducts and impurities therefore increase, cost, and complexity of biosynthesizing compounds and can decrease efficiency or yield of the desired products.

### SUMMARY

Provided herein are non-naturally occurring engineered trans-enoyl CoA reductase (TER) enzyme capable of converting 5-carboxy-2-pentenoyl-CoA (CPCoA) substrate to form adipyl-CoA product.

Also provided herein are non-naturally occurring microbial organisms having a 6-aminocaproic acid pathway, caprolactam pathway, hexamethylenediamine pathway, caprolactone pathway, 1,6-heaxanediol pathway, or a combination of one or more of these pathways. The non-naturally occurring microbial organisms comprise at least one exogenous nucleic acid encoding a TER enzyme that reacts with 5-carboxy-2-pentenoyl-CoA (CPCoA) substrate to form adipylCoA. The TER enzyme has greater activity, greater catalytic efficiency, or a combination thereof for CPCoA substrate as compared to crotonylCoA.

The non-naturally occurring microbial organisms may further comprise additional exogenous nucleic acids encoding enzymes necessary for producing 6-aminocaproic acid, 1,6-hexanediol, caprolactone, caprolactam, hexamethylenediamine in a sufficient amount to produce the respective product. In some cases, one or more of these exogenous nucleic acids may be heterologous to the microbial organisms.

Also disclosed are methods for producing 6-aminocaproic acid, 1,6-hexanediol, caprolactone, caprolactam, hexamethylenediamine. The methods can include culturing a 6-aminocaproic acid, 1,6-hexanediol, caprolactone, caprolactam, and/or hexamethylenediamine producing non-naturally occurring microbial organisms, where the microbial organisms express at least one exogenous nucleic acid encoding a trans-enoylCoA reductase enzyme that reacts with with 5-carboxy-2-pentenoyl-CoA (CPCoA) substrate to form adipylCoA. The methods include culturing the non-naturally occurring microbial organisms under conditions and for a sufficient period of time to produce 6-aminocaproic acid, 1,6-hexanediol, caprolactone, caprolactam, hexamethylenediamine.

In one aspect is a TER comprising an amino acid sequence having at least 50% sequence identity to at least 25 or more contiguous amino acids of any of SEQ ID NO:1, 4-9, 11, 14, 15, or 17-26.

In one aspect, an engineered TER enzyme comprises at least one alteration of an amino of SEQ ID NO:1 or SEQ ID NO: 24. In some embodiments, an engineered TER enzyme comprises one or more amino acid alterations at one or more amino acid positions of SEQ ID NO:1 or SEQ ID NO: 24. In some embodiments, the engineered TER enzyme comprises at least two, three, four, five, six, seven, eight, or more amino acid substitutions corresponding to the related wild-type sequence. In some embodiments, the amino acid substitutions are conservative substitutions. In some embodiments, the amino acid substitutions are non-conservative substitutions. In some embodiments, the engineered TER has greater catalytic efficiency for 5-carboxyl-2-pentenoyl-CoA as the substrate compared to crotonyl-CoA as the substrate.

In one aspect, the engineered TER has one or more amino acid alterations at one or more amino acid positions corresponding to residues Q11, A39, S48, Q52, S59, G97, S103, H104, V105, N106, F107, I147, S148, V149, L152, L156, R200, D201, R202, V301, T302, E303, K306, N307, N308, L316, or combinations thereof of the amino acid alterations and amino acid postions of SEQ ID NO:1. In some embodiments, the engineered TER has one or more amino acid substitutions disclosed in Tables 5 and 6F below.

In one aspect, the engineered TER has alterations of the amino acids selected from positions corresponding to residues S96, L98, M274, T275 of SEQ ID NO: 24. In some embodiments, the engineered TER comprises one or more amino acid substitutions: S96G, L98M, M274R, T275R corresponding to SEQ ID NO: 24.

In one aspect the selected and engineered TERs reacts with 5-carboxyl-2-pentenoyl-CoA to form adipyl-CoA. In some embodiments, the engineered TER comprises activity that is at least 20% higher than the activity of the trans-enoyl CoA reductase of SEQ ID NO:1 or SEQ ID NO; 24 with no amino acid alterations.

In one aspect, the TER enzyme preferentially uses NADH as a cofactor. In other aspects the TER enzyme preferentially uses NADPH as a cofactor.

In one aspect is a non-naturally occurring microbial organism comprising at least one exogenous enzyme, the at least one exogenous enzyme comprising a TER converting 5-carboxyl-2-pentenoyl-CoA to adipyl CoA, the TER selected from:
(i) a TER comprising the amino acid sequence of SEQ ID NO:1, 4-9, 11, 14, 15, or 17-26; (ii) comprising an amino acid sequence having at least 50% sequence identity to at least 25 or more contiguous amino acids of any of SEQ ID NO:1, 4-9, 11, 14, 15, or 17-26; and (iii) an engineered TER of SEQ ID NO; 1 or 24.

In some embodiments, the non-naturally occurring microbial organism comprising an exogenous enzyme encoding a TER further comprises genes encoding a 3-oxoadipyl-CoA thiolase, a 3-oxoadipyl-CoA dehydrogenase, and a 3-oxoadipyl-CoA dehydratase. In some embodiments, the non-naturally occurring microbial organism is able to produce at least 10% more adipate than a control cell that expresses the wild type TER.

In one aspect, provided are recombinant nucleic acid molecules comprising a nucleic acid sequence encoding an engineered TER. In some embodiments, the engineered TER is operably linked to a heterologous promoter. In some embodiments, a vector comprises the recombinant nucleic acid molecule.

In one aspect, provided are methods of producing adipyl CoA comprising culturing a non-naturally occurring microorganism of any one of the above aspects and embodiments for a sufficient time period and conditions for producing adipyl CoA.

In one aspect, provided are methods of producing 6-aminocaproic acid (6ACA) comprising culturing a non-naturally occurring microbial organism of any one the above aspect and embodiments for a sufficient time period and conditions for producing 6ACA. In some embodiments, the methods further include recovering 6ACA from the microbial organism, fermentation broth, or both.

In one aspect provided are methods of producing hexamethylene diamine comprising culturing a non-naturally occurring microbial organism of any one of the above aspects and embodiments for a sufficient time period and conditions for producing hexamethylene diamine. In some embodiments, the methods further include recovering hexamethylene diamine from the microbial organism, fermentation broth, or both. In some embodiments, the non-naturally occurring microbial organism comprises two, three, four, five, six or seven exogenous nucleic acid sequences each encoding a hexamethylene diamine pathway enzyme.

In one aspect, provided are methods of producing 6-aminocaproic acid, 1,6-hexanediol, caprolactone, caprolactam, hexamethylenediamine comprising culturing a non-naturally occurring microbial organism of any one of the above aspects and embodiments for a sufficient time period and conditions for producing 6-aminocaproic acid, 1,6-hexanediol, caprolactone, caprolactam, hexamethylenediamine. In some embodiments, the methods further include recovering caprolactam from the microbial organism, fermentation broth, or both. In some embodiments, the non-naturally occurring microbial organism comprises two, three, four, five, six or seven exogenous nucleic acid sequences each encoding 6-aminocaproic acid, 1,6-hexanediol, caprolactone, caprolactam, hexamethylenediamine pathway enzymes.

In one aspect, provided are bioderived 6-aminocaproic acid, hexamethylenediamine, or caprolactam synthesized using the above disclosed methods.

In some embodiments, the TER enzyme of the non-naturally occurring microbial organism has higher catalytic efficiency for CPCoA substrate compared to crotonyl-CoA substrate. In some embodiments, the catalytic efficiency of the TER enzyme for CPCoA substrate is at least twice as high as the catalytic efficiency for crotonyl-CoA substrate.

In some embodiments, the aldehyde dehydrogenase enzyme of the non-naturally occurring microbial organism further reacts with 6-aminocaproyl-CoA to form 6-aminocaproate semialdehyde.

In some embodiments, the non-naturally occurring microbial organism comprising at least one exogenous nucleic acid encoding a TER enzyme converts more CPCoA to adipylCoA than a control microbial organism substantially identical to the non-naturally occurring microbial organism, with the exception that the control microbial organism does not comprise the exogenous nucleic acid encoding a TER enzyme.

In some embodiments, the non-naturally occurring microbial organism comprising at least one exogenous nucleic acid encoding a TER enzyme converts more CPCoA to adipylCoA than its wild-type microbial organism substantially identical to the non-naturally occurring microbial organism, with the exception that the wild-type microbial organism does not comprise the exogenous nucleic acid encoding an engineered TER enzyme.

In some embodiments, at least one exogenous nucleic acid encoding TER enzyme that reacts with CPCoA to form adipyl-CoA is heterologous to the microbial organism.

In some embodiments, the non-naturally occurring microbial organism comprises a 6-aminocaproic acid pathway. In some embodiments, the 6-aminocaproic acid pathway comprises: (i) transaminase, (ii) 6-aminocaproate dehydrogenase, or both (iii) transaminase and 6-aminocaproate dehydrogenase enzymes. In some embodiments, the non-naturally occurring microbial organism further comprises one or more additional exogenous nucleic acids encoding one or more of the 6-aminocaproic acid pathway enzymes. In some embodiments, the exogenous nucleic acids encoding one or more of the 6-aminocaproic acid pathway enzymes are heterologous to the microbial organism.

In some embodiments, the non-naturally occurring microbial organism comprises a hexamethylenediamine pathway. In some embodiments, the hexamethylenediamine pathway comprises (i) 6-aminoacaproyl CoA transferase, (ii) 6-amino caproyl CoA synthase, (iii) 6-amino caproyl CoA reductase, (iv) hexamethylenediamine transaminase, (v) hexamethylenediamine dehydrogenase, (v) or a combination of one or more of the enzymes (i)-(v). In some embodiments, the microbial organism further comprises one or more additional exogenous nucleic acids encoding one or more of the hexamethylenediamine pathway enzymes such as carboxylic acid reductase (CAR) that converts 6-aminocaproate to 6-aminocaproate semialdehyde. The 6-aminocaproate semialdehyde can subsequently be converted to hexamethylene diamine. In some embodiments, the exogenous nucleic acids encoding one or more of the hexamethylenediamine pathway enzymes are heterologous to the microbial organism.

In some embodiments, the non-naturally occurring microbial organism comprises a caprolactam pathway. In some embodiments, the caprolactam pathway comprises aminohydrolase enzyme. In some embodiments, the microbial organism further comprises one or more additional exogenous nucleic acids encoding aminohydrolase enzyme. In some embodiments, the exogenous nucleic acids encoding aminohydrolase enzyme is heterologous to the microbial organism.

In some embodiments, the non-naturally occurring microbial organism comprises a 1, 6-hexanediol pathway. In some embodiments, the 1, 6-hexanediol pathway comprises the following enzymes: a 6-aminocaproyl-CoA transferase or synthetase catalyzing conversion of 6ACA to 6-aminocaproyl-CoA; a 6-aminocaproyl-CoA reductase catalyzing conversion of 6-aminocaproyl-CoA to 6-aminocaproate semialdehyde; a 6-aminocaproate semialdehyde reductase catalyzing conversion of 6-aminocaproate semialdehyde to 6-aminohexanol; a 6-aminocaproate reductase catalyzing conversion of 6ACA to 6-aminocaproate semialdehyde; an adipyl-CoA reductase adipyl-CoA to adipate semialdehyde; an adipate semialdehyde reductase catalyzing conversion of adipate semialdehyde to 6-hydroxyhexanoate; a 6-hydroxyhexanoyl-CoA transferase or synthetase catalyzing conversion of 6-hydroxyhexanoate to 6-hydroxyhexanoyl-CoA; a 6-hydroxyhexanoyl-CoA reductase catalyzing conversion of 6-hydroxyhexanoyl-CoA to 6-hydroxyhexanal; a 6-hydroxyhexanal reductase catalyzing conversion of 6-hydroxyhexanal to HDO; a 6-aminohexanol aminotransferase or oxidoreductases catalyzing conversion of 6-aminohexanol to 6-hydroxyhexanal; a 6-hydroxyhexanoate reductase catalyzing conversion of 6-hydroxyhexanoate to 6-hydroxyhexanal; an adipate reductase catalyzing conversion of ADA to adipate semialdehyde; and an adipyl-CoA transferase, hydrolase or synthase catalyzing conversion of adipyl-CoA to ADA.

In some embodiments, the non-naturally occurring microbial organism comprises pathways from adipate or adipyl-CoA to caprolactone. In some embodiments, the pathways from adipate or adipyl-CoA to caprolactone comprise the following enzymes: adipyl-CoA reductase, adipate semialdehyde reductase, 6-hydroxyhexanoyl-CoA transferase or synthetase, 6-hydroxyhexanoyl-CoA cyclase or spontaneous cyclization, adipate reductase, adipyl-CoA transferase, synthetase or hydrolase, 6-hydroxyhexanoate cyclase, 6-hydroxyhexanoate kinase, 6-hydroxyhexanoyl phosphate cyclase or spontaneous cyclization, phosphotrans-6-hydroxyhexanoylase.

In some embodiments, the TER of the non-naturally occurring microbial organism is derived from a prokaryotic species. In some embodiments, the TER enzyme is derived species shown in Table 3.

In some embodiments, the non-naturally occurring microbial organism comprises a species of Acinetobacter, Actinobacillus, Anaerobiospirillum, Aspergillus, Bacillus, Clostridium, Corynebacterium, Escherichia, Gluconobacter, Klebsiella, Kluyveromyces, Lactococcus, Lactobacillus, Mannheimia, Pichia, Pseudomonas, Rhizobium, Rhizopus, Saccharomyces, Schizosaccharomyces, Streptomyces, and Zymomonas. In some embodiments, the non-naturally occurring microbial organism is a strain of Escherichia. coli.

In some embodiments, the culturing is performed in a fermentation broth comprising a sugar.

### BRIEF DESCRIPTION OF DRAWINGS

FIG. 1 shows exemplary pathways from succinyl-CoA and acetyl-CoA to 6-aminocaproate, hexamethylenediamine (HMDA), and caprolactam. The enzymes are designated as follows: A) 3-oxoadipyl-CoA thiolase, B) 3-oxoadipyl-CoA reductase, C) 3-hydroxyadipyl-CoA dehydratase, D) 5-carboxy-2-pentenoyl-CoA reductase, E) 3-oxoadipyl-CoA/acyl-CoA transferase, F) 3-oxoadipyl-CoA synthase, G) 3-oxoadipyl-CoA hydrolase, H) 3-oxoadipate reductase, I) 3-hydroxyadipate dehydratase, J) 5-carboxy-2-pentenoate reductase, K) adipyl-CoA/acyl-CoA transferase, L) adipyl-CoA synthase, M) adipyl-CoA hydrolase, N) adipyl-CoA reductase (Aldehyde forming), 0) 6-aminocaproate transaminase, P) 6-aminocaproate dehydrogenase, Q) 6-aminocaproyl-CoA/acyl-CoA transferase, R) 6-aminocaproyl-CoA synthase, S) amidohydrolase, T) spontaneous cyclization, U) 6-aminocaproyl-CoA reductase (Aldehyde forming), V) HMDA transaminase, W) HMDA dehydrogenase, X) adipate reductase, Y) adipate kinase, Z) adipylphosphate reductase.

FIG. 2 is a graphical representation of the in vivo adipate production of the wild-type SEQ ID NO: 1 to variants of SEQ ID NO: 1. The in vivo adipate concentration is in nM.

FIG. 3 shows the TER amino acid sequence alignment of SEQ ID NO: 1 (Candida) and SEQ ID NO: 24 (Drosophila).

FIG. 4 shows an exemplary pathway for synthesis of 6-amino caproic acid and adipate using lysine as a starting point.

FIG. 5 shows an exemplary caprolactam synthesis pathway using adipyl-CoA as a starting point.

FIG. 6 shows exemplary pathways to 6-aminocaproate from pyruvate and succinic semialdhyde. Enzymes are A) HODH aldolase, B) OHED hydratase, C) OHED reductase, D) 2-OHD decarboxylase, E) adipate semialdhyde aminotransferase and/or adipate semialdhyde oxidoreductase (aminating), F) OHED decarboxylase, G) 6-OHE reductase, H) 2-OHD aminotransferase and/or 2-OHD oxidoreductase (aminating), I) 2-AHD decarboxylase, J) OHED aminotransferase and/or OHED oxidoreductase (aminating), K) 2-AHE reductase, L) HODH formate-lyase and/or HODH dehydrogenase, M) 3-hydroxyadipyl-CoA dehydratase, N) 2,3-dehydroadipyl-CoA reductase, 0) adipyl-CoA dehydrogenase, P) OHED formate-lyase and/or OHED dehydrogenase, Q) 2-OHD formate-lyase and/or 2-OHD dehydrogenase. Abbreviations are: HODH=4-hydroxy-2-oxoheptane-1,7-dioate, OHED=2-oxohept-4-ene-1,7-dioate, 2-OHD=2-oxoheptane-1,7-dioate, 2-AHE=2-aminohept-4-ene-1,7-dioate, 2-AHD=2-aminoheptane-1,7-dioate, and 6-OHE=6-oxohex-4-enoate.

FIG. 7 shows exemplary pathways to hexamethylenediamine from 6-aminocapropate. Enzymes are A) 6-aminocaproate kinase, B) 6-AHOP oxidoreductase, C) 6-aminocaproic semialdhyde aminotransferase and/or 6-aminocaproic semialdhyde oxidoreductase (aminating), D) 6-aminocaproate N-acetyltransferase, E) 6-acetamidohexanoate kinase, F) 6-AAHOP oxidoreductase, G) 6-acetamidohexanal aminotransferase and/or 6-acetamidohexanal oxidoreductase (aminating), H) 6-acetamidohexanamine N-acetyltransferase and/or 6-acetamidohexanamine hydrolase (amide), I) 6-acetamidohexanoate CoA transferase and/or 6-acetamidohexanoate CoA ligase, J) 6-acetamidohexanoyl-CoA oxidoreductase, K) 6-AAHOP acyltransferase, L) 6-AHOP acyltransferase, M) 6-aminocaproate CoA transferase and/or 6-aminocaproate CoA ligase, N) 6-aminocaproyl-CoA oxidoreductase. Abbreviations are: 6-AAHOP=[(6-acetamidohexanoyl)oxy]phosphonate and 6-AHOP=[(6-aminohexanoyl)oxy]phosphonate.

FIG. 8 shows exemplary biosynthetic pathways leading to 1,6-hexanediol. A) is a 6-aminocaproyl-CoA transferase or synthetase catalyzing conversion of 6ACA to 6-aminocaproyl-CoA; B) is a 6-aminocaproyl-CoA reductase catalyzing conversion of 6-aminocaproyl-CoA to 6-aminocaproate semialdehyde; C) is a 6-aminocaproate semialdhyde reductase catalyzing conversion of 6-aminocaproate semialdhyde to 6-aminohexanol; D) is a 6-aminocaproate reductase catalyzing conversion of 6ACA to 6-aminocaproate semialdhyde; E) is an adipyl-CoA reductase adipyl-CoA to adipate semialdhyde; F) is an adipate semialdhyde reductase catalyzing conversion of adipate semialdhyde to 6-hydroxyhexanoate; G) is a 6-hydroxyhexanoyl-CoA transferase or synthetase catalyzing conversion of 6-hydroxyhexanoate to 6-hydroxyhexanoyl-CoA; H) is a 6-hydroxyhexanoyl-CoA reductase catalyzing conversion of 6-hydroxyhexanoyl-CoA to 6-hydroxyhexanal; I) is a 6-hydroxyhexanal reductase catalyzing conversion of 6-hydroxyhexanal to HDO; J) is a 6-aminohexanol aminotransferase or oxidoreductases catalyzing conversion of 6-aminohexanol to 6-hydroxyhexanal; K) is a 6-hydroxyhexanoate reductase catalyzing conversion of 6-hydroxyhexanoate to 6-hydroxyhexanal; L) is an adipate reductase catalyzing conversion of ADA to adipate semialdhyde; and M) is an adipyl-CoA transferase, hydrolase or synthase catalyzing conversion of adipyl-CoA to ADA.

FIG. 9 shows exemplary pathways from adipate or adipyl-CoA to caprolactone. Enzymes are A. adipyl-CoA reductase, B. adipate semialdhyde reductase, C. 6-hydroxyhexanoyl-CoA transferase or synthetase, D. 6-hydroxyhexanoyl-CoA cyclase or spontaneous cyclization, E. adipate reductase, F. adipyl-CoA transferase, synthetase or hydrolase, G. 6-hydroxyhexanoate cyclase, H. 6-hydroxyhexanoate kinase, I. 6-hydroxyhexanoyl phosphate cyclase or spontaneous cyclization, J. phosphotrans-6-hydroxyhexanoylase.

### DETAILED DESCRIPTION

Unless defined otherwise, all technical and scientific terms used herein have the same meaning as commonly understood by those of ordinary skill in the art to which the invention belongs. Any methods, devices and materials similar or equivalent to those described herein can be used in the practice of this invention. The following definitions are provided to facilitate understanding of certain terms used frequently herein and are not meant to limit the scope of the present disclosure. All references referred to herein are incorporated by reference in their entirety.

Trans-enoyl CoA reductase (TER) enzymes can reduce a 5-carboxy-2-pentenoyl-CoA (CPCoA) substrate to form adiplyCoA. This enzyme maybe used in various pathways leading to nylon intermediates. However, many TERs have low activity on CPCoA, can reduce longer chain carbon enoyl-CoA substrates, and can react with crotonyl-CoA to produce butryl-CoA.

Described are TER enzymes that were identified to be active on CPCoA. In some embodiments, the disclosed TER enzyme can use CPCoA and and crotonyl-CoA as substrates. In some embodiments, disclosed TER enzymes are non-natural variants. In some embodiments, the variant TER enzymes have higher affinity CPCoA as substrate as compared to their wild-type counterpart.

In one embodiment, a TER enzyme from Candida topicalis encoded by the Ter gene (SEQ ID NO: 1) was identified. To identify other TER enzymes, SEQ ID NO: 1 was used. Homologous enzymes were identified as set out in Table 3.

In some embodiments, some identified TERs are engineered to have greater catalytic efficiency and activity for the 5-carboxy-2-pentenoyl-CoA (CPCoA) substrate compared to the related wild-type enzyme (viz., unmodified enzyme). In some embodiments, the TER has greater catalytic efficacy and activity for the 5-carboxy-2-pentenoyl-CoA substrate as compared to crotonyl CoA substrate. This improves the efficiency and in turn the production of the nylon intermediates.

Also described are non-naturally occurring microbial organisms engineered to express exogenous TER enzymes having greater catalytic efficiency and activity for the 5-carboxy-2-pentenoyl-CoA substrate as compared to its unmodified, wild-type TER (e.g. SEQ ID NO: 1 or 24) or greater catalytic efficiency and activity for the CPCoA substrate compared to crotonyl-CoA.

In some embodiments, 5-trans-enoyl CoA reductase enzymes or sequences are identified by BLAST. In some embodiments, the trans-enoyl CoA reductases share at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences of the TERs of Table 3.

In some embodiments, 5-trans-enoyl CoA reductase enzymes or sequences are identified by BLAST. In some embodiments, the trans-enoyl CoA reductases share at least about 40%, 45%, 50%, 55%, 60%, 65%, 70%, 75%, 80%, 85%, 90%, 95%, 96%, 97%, 98%, 99% or 100% sequence identity, to at least 25, 50, 75, 100, 150, 200, 250, 300 or more contiguous amino acids of the amino acid sequences SEQ ID NO: 1, 4-9, 11, 14, 15, or 17-26.

The trans-enoyl CoA reductase enzymes have conserved domains. Based on the multiple sequence alignments and hidden Markov models (HMMs), the TER enzymes are grouped into Pfam PF00107 of the Pfam database from the European Bioinformatics Institute (pfam.xfam.org).

In some embodiments, the engineered TER enzymes have greater catalytic efficiency, and/or activity when 5-carboxy-2-pentenoyl-CoA is the substrate as compared to crotonyl-CoA. In some embodiments, the trans-enoyl CoA reductase enzyme comprises an amino acid sequence having at least about 50% amino acid sequence identity to at least 25, 50, 75, 100, 150, 200, 250, 300, or more contiguous amino acids of any of SEQ ID NOs: 1, 4-9, 11, 14, 15, or 17-26. In some embodiments the amino acid sequence of the trans-enoyl CoA reductase enzyme that reacts with 5-carboxy-2-pentenoyl-CoA to form adipyl-CoA are selected from the amino acid sequences of SEQ ID NOs: 1, 4-9, 11, 14, 15, or 17-26.

In some embodiments, the TER enzyme has at least a catalytic efficiency for 5-carboxy-2-pentenoyl-CoA substrate that is at least 1.5×, at least 2.5×, 5×, at least 10×, at least 25×, or 1.5-25× as compared to crotonyl-CoA substrate.

In some embodiments, the enzymatic conversion of 5-carboxy-2-pentenoyl-CoA by trans-enoyl CoA reductase enzyme under known standard conditions is at least 10, at least 20, at least 30, at least 40, at least 50, at least 60, at least 70, at least 80, or at least 90 percent more than the enzymatic activity of the enzyme for crotonyl-CoA.

In some embodiments, the trans-enoyl CoA reductase enzyme is engineered to have greater specificity for the CPCoA substrate than its corresponding wild-type sequence or greater specificity for the CPCoA compared to crotonyl-CoA substrate.

As used herein “engineered” or “variant” when used in the context of a polypeptide or nucleic acid refers to a sequence having at least one variation or alteration at an amino acid position or nucleic acid position as compared to an unmodified, wild-type sequence.

In some embodiments, the engineered TERS have at least one alteration of an amino of SEQ ID NO: 1 or SEQ ID NO: 24.

In some embodiments, amino acid positions were identified for mutation in SEQ ID NO: 1 or 24 by examination of the crystal structure of the protein, and the gene encoding SEQ ID NO:1 or 24 were subjected to saturation mutagenesis at selected amino acid positions. Catalytically-relevant residues were identified that can be subject to change to provide a variant amino acid with activity better than the wild-type (unmodified) SEQ ID NO: 1 or SEQ ID NO: 24.

In some embodiments, trans-enoyl CoA reductase enzyme is engineered to have greater specificity for the CPCoA substrate than its corresponding wild-type. The engineered TER having greater specificity for CPCoA substrate than its corresponding wild-type are variants of SEQ ID NO: 1 and are numbered variant number 2-69, 85,100-103, 106-111 or combinations thereof. In some embodiments, the engineered TER having greater specificity for CPCoA substrate than its corresponding wild-type are variants of SEQ ID NO: 1 and are numbered variant number 2, 3, 4, 9, 19, 22, 23, 32, 37, 38, 39, 40, 46, 48, 51, 53, 85, 100, 101, 102, 103, 106, 107, 108, 109, 110 or 111 or combinations thereof.

In some embodiments, the engineered trans-enoyl CoA reductase enzyme has one or more amino acids replaced at one or more amino acid positions as shown in Tables 5 and 6 or combinations thereof of the amino acid positions and amino acid alterations.

In some embodiments, the TER is NADPH preferring and selected from variant numbers 2-68, 106, 11 or combinations thereof of SEQ ID NO: 1.

In some embodiments, the TER is NADH preferring and selected from variant numbers 85, 100-105, 107, 108, or combinations thereof of SEQ ID NO: 1

In some embodiments the engineered TERS have alterations in amino acid sequences that have at least one, two, at least three, at least four, at least five, at least six, at least seven, or at least eight alterations of an amino acid with respect to SEQ ID NO:1 or SEQ ID NO; 24.

In some embodiments, the engineered TER have at least one amino acid alteration selected from one or more positions corresponding to residues Q11, A39, S48, Q52, S59, G97, S103, H104, V105, N106, F107, I147, S148, V149, L152, L156, R200, D201, R202, V301, T302, E303, K306, N307, N308, L316, and combinations thereof of the amino acid positions and amino acid alterations of SEQ ID NO:1.

In some embodiments, the engineered TER have one or more amino acid alterations selected from positions corresponding to residues Q11, A39, S48, Q52, S59, G97, S103, H104, V105, N106, F107, I147, S148, V149, L152, L156, R200, D201, R202, V301, T302, E303, K306, N307, N308, and L316 or combinations thereof of SEQ ID NO:1.

In some embodiments the one amino acid alteration of the engineered protein is a substitution of a conservative or non-conservative amino acid at a position corresponding to residue Q11, A39, S48, Q52, S59, G97, S103, H104, V105, N106, F107, I147, S148, V149, L152, T153, L156, R200, D201, R202, V301, T302, E303, K306, N307, N308, L316 or combinations thereof of SEQ ID NO:1.

In some embodiments, the engineered TER has one or more amino acid alterations of SEQ ID NO: 1 at a positions corresponding to residues shown in Table 5 and 6 and combinations thereof.

In some embodiments, the engineered TER enzyme has at least a catalytic efficiency for 5-carboxy-2-pentenoyl-CoA substrate that is at least 1.5×, at least 2.5×, 5×, at least 10×, at least 25×, or 1.5-25× as compared to crotonyl-CoA substrate or the corresponding wild-type of SEQ ID NO: 1 and SEQ ID NO: 24.

In some embodiments, the enzymatic conversion of 5-carboxy-2-pentenoyl-CoA by the engineered trans-enoyl CoA reductase enzyme under known standard conditions is at least 10, at least 20, at least 30, at least 40, at least 50, at least 60, at least 70, at least 80, or at least 90 percent more than the enzymatic activity of the enzyme for crotonyl-CoA or the corresponding wild-type of SEQ ID NO: 1 and SEQ ID NO: 24.

In some embodiments, to provide a TER variant, Candida tropicalis TER represented by SEQ ID NO: 1 of the disclosure is selected as a template. Variants, as described herein, can be created by introducing into the template one or more amino acid alterations (e.g. substitutions) to test for increased activity and increased specificity to CPCoA or analogs thereof.

For the purpose of amino acid position numbering, in some embodiments, SEQ ID NO: 1 is used as the reference sequence. Therefore, for example, mention of amino acid position 79 in reference to SEQ ID NO:1, but in the context of a different TER sequence (a target sequence or other template sequence) the corresponding amino acid position for variant creation may have the same or different position number, (e.g. 78, 79 or 80). In some cases, the original amino acid and its position on the SEQ ID NO: 1 reference template will precisely correlate with the original amino acid and position on the target TER. In other cases, the original amino acid and its position on the SEQ ID NO: 1 template will correlate with the original amino acid, but its position on the target will not be in the corresponding template position. However, the corresponding amino acid on the target can be a predetermined distance from the position on the template, such as within 10, 9, 8, 7, 6, 5, 4, 3, 2, or 1 amino acid positions from the template position. In other cases, the original amino acid on the SEQ ID NO: 1 template will not precisely correlate with the original amino acid on the target. However, one can understand what the corresponding amino acid on the target sequence is based on the general location of the amino acid on the template and the sequence of amino acids in the vicinity of the target amino acid. It is understood that additional alignments can be generated with TER sequences not specifically disclosed herein, and such alignments can be used to understand and generate new TER variants in view of the current disclosure. In some modes of practice, the alignments can allow one to understand common or similar amino acids in the vicinity of the target amino acid, and those amino acids may be viewed as “sequence motif” having a certain amount of identity or similarity to between the template and target sequences. Those sequence motifs can be used to describe portions of TER sequences where variant amino acids are located, and the type of variation(s) that can be present in the motif.

In some cases, it can be useful to use the Basic Local Alignment Search Tool (BLAST) algorithm to understand the sequence identity between an amino acid motif in a template sequence and a target sequence. Therefore, in preferred modes of practice, BLAST is used to identify or understand the identity of a shorter stretch of amino acids (e.g. a sequence motif) between a template and a target protein. BLAST finds similar sequences using a heuristic method that approximates the Smith-Waterman algorithm by locating short matches between the two sequences. The (BLAST) algorithm can identify library sequences that resemble the query sequence above a certain threshold. Exemplary parameters for determining relatedness of two or more sequences using the BLAST algorithm, for example, can be as set forth below. Briefly, amino acid sequence alignments can be performed using BLASTP version 2.0.8 (Jan. 5 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

Site-directed mutagenesis or sequence alteration (e.g., site-specific mutagenesis or oligonucleotide-directed) can be used to make specific changes to a target TER DNA sequence to provide a variant DNA sequence encoding TER with the desired amino acid substitution. As a general matter, an oligonucleotide having a sequence that provides a codon encoding the variant amino acid is used. In some embodiments, artificial gene synthesis of the entire coding region of the variant TER DNA sequence is performed as preferred TER targeted for substitution are generally less than 150 amino acids long.

Exemplary techniques using mutagenic oligonucleotides for generation of a variant TER sequence include the Kunkel method which may utilize a TER gene sequence placed into a phagemid. The phagemid in E. coli TA ssDNA which is the template for mutagenesis using an oligonucleotide which is a primer extended on the template.

Depending on the restriction enzyme sites flanking a location of interest in the TER DNA, cassette mutagenesis may be used to create a variant sequence of interest. For cassette mutagenesis, a DNA fragment is synthesized inserted into a plasmid, cleaved with a restriction enzyme, and then subsequently ligated to a pair of complementary oligonucleotides containing the TER variant mutation. The restriction fragments of the plasmid and oligonucleotide can be ligated to one another.

In other embodiments, another technique used to generate the variant TER sequence is PCR site directed mutagenesis. Mutagenic oligonucleotide primers are used to introduce the desired mutation and to provide a PCR fragment carrying the mutated sequence. Additional oligonucleotides may be used to extend the ends of the mutated fragment to provide restriction sites suitable for restriction enzyme digestion and insertion into the gene.

Commercial kits for site-directed mutagenesis techniques are also available. For example, the Quikchange™ kit uses complementary mutagenic primers to PCR amplify a gene region using a high-fidelity non-strand-displacing DNA polymerase such as pfu polymerase. The reaction generates a nicked, circular DNA which is relaxed. The template DNA is eliminated by enzymatic digestion with a restriction enzyme such as DpnI which is specific for methylated DNA.

In some embodiments, an optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >104). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol. Eng 22:1-9 (2005); and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Km), including broadening substrate binding to include non-natural substrates; inhibition (Ki), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well-known to those skilled in the art. Any of these can be used to alter and/or optimize the activity of a 6-amino caproic acid, hexamethylenediamine or capralactam pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e17 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional ts mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

A cell having the desired enzymatic activity can be identified using any method known in the art. For example, enzyme activity assays can be used to identify cells having enzyme activity, see, for example, Enzyme Nomenclature, Academic Press, Inc., New York 2007. Other assays that may be used to determine reaction between of TER on CPCoA include GC/MS analysis. In other examples, levels of NADH/NADPH may be monitored. For example, the NADH/NADPH may be monitored colorimetrically or spectroscopically using NADP/NADPH assay kits (e.g. ab65349 available from ABCAM™).

The disclosed TER enzyme can be used in pathways for the production of the nylon intermediates. In some embodiments, a non-naturally occurring microorganism may be used in the production of adipate semialdehyde or other nylon intermediates that are produced using the adipate semialdehyde as an intermediate.

In some embodiments, genetically modified cells (e.g. non-naturally occurring microorganisms) are capable of producing the nylon intermediates such as 6-aminocaproic acid, caprolactam, and hexamethylenediamine.

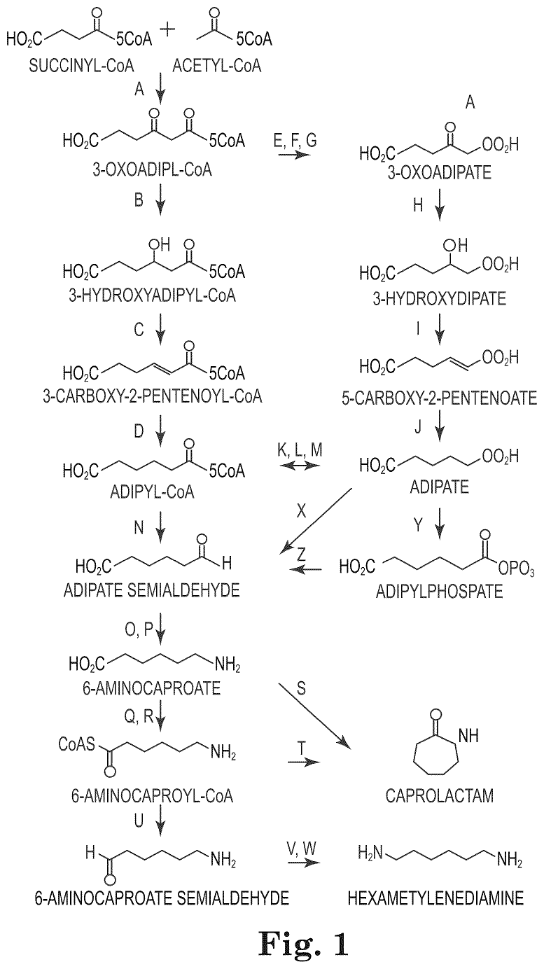

In some embodiments, the nylon intermediates are biosynthesized using the pathway described in FIG. 1. In some embodiments, FIG. 1 pathway is provided in genetically modified cell described herein (e.g., a non-naturally occurring microorganism) where the pathway includes at least one exogenous nucleic acid encoding a pathway enzyme expressed in a sufficient amount to produce 6-aminocaproic acid, caprolactam, and hexamethylenediamine.

In some embodiments the pathway is an HMD pathway as set forth in FIG. 1. The HMD pathway is provided in genetically modified cell described herein (e.g., a non-naturally occurring microorganism) where the HMD pathway includes at least one exogenous nucleic acid encoding a HMD pathway enzyme expressed in a sufficient amount to produce HMD. The enzymes are 1A is a 3-oxoadipyl-CoA thiolase; 1B is a 3-oxoadipyl-CoA reductase; 1C is a 3-hydroxyadipyl-CoA dehydratase; 1D is a 5-carboxy-2-pentenoyl-CoA reductase; 1E is a 3-oxoadipyl-CoA/acyl-CoA transferase; 1F is a 3-oxoadipyl-CoA synthase; 1G is a 3-oxoadipyl-CoA hydrolase; 1H is a 3-oxoadipate reductase; 1I is a 3-hydroxyadipate dehydratase; 1J is a 5-carboxy-2-pentenoate reductase; 1K is an adipyl-CoA/acyl-CoA transferase; 1L is an adipyl-CoA synthase; 1M is an adipyl-CoA hydrolase; 1N is an adipyl-CoA reductase (Aldehyde forming); 10 is a 6-aminocaproate transaminase; 1P is a 6-aminocaproate dehydrogenase; 1Q is a 6-aminocaproyl-CoA/acyl-CoA transferase; 1R is a 6-aminocaproyl-CoA synthase; 1S is an amidohydrolase; 1T is spontaneous cyclization; 1U is a 6-aminocaproyl-CoA reductase (Aldehyde forming); 1V is a HMDA transaminase; and 1W is a HMDA dehydrogenase.

With reference to FIG. 1, in some embodiments, the non-naturally occurring microorganism has one or more of the following pathways: ABCDNOPQRUVW; ABCDNOPQRT; or: ABCDNOPS. Other exemplary pathways that include the TER enzyme to produce adipate semialdhyde include those described in U.S. Pat. No. 8,377,680 incorporated herein by reference in their entireties.

FIG. 1 also shows a pathway from 6-aminocaproate to 6-aminocaproyl-CoA by a transferase or synthase enzyme (FIG. 1, Step Q or R) followed by the spontaneous cyclization of 6-aminocaproyl-CoA to form caprolactam (FIG. 1, Step T). In other embodiments, 6-aminocaproate is activated to 6-aminocaproyl-CoA (FIG. 1, Step Q or R), followed by a reduction (FIG. 1, Step U) and amination (FIG. 1, Step V or W) to form HMDA. 6-Aminocaproic acid can also be activated to 6-aminocaproyl-phosphate instead of 6-aminocaproyl-CoA. 6-Aminocaproyl-phosphate can spontaneously cyclize to form caprolactam. In some embodiments, 6-aminocaproyl-phosphate can be reduced to 6-aminocaproate semialdehyde, which can be then converted to HMDA as depicted in FIG. 1.

As used herein, the term “non-naturally occurring” when used in reference to a microbial organism or microorganism is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes within a 6-aminocaproic acid, caprolactam, hexamethylenediamine or levulinic acid biosynthetic pathway.

A metabolic modification refers to a biochemical reaction that is altered from its naturally occurring state. Therefore, non-naturally occurring microorganisms can have genetic modifications to nucleic acids encoding metabolic polypeptides or, functional fragments thereof. Exemplary metabolic modifications are disclosed herein.

As used herein, the terms “microbial,” “microbial organism” or “microorganism” has been used interchangeably and is intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, “adipate,” having the chemical formula —OOC—(CH2)4-COO— (see FIG. 1) (IUPAC name hexanedioate), is the ionized form of adipic acid (IUPAC name hexanedioic acid), and it is understood that adipate and adipic acid can be used interchangeably throughout to refer to the compound in any of its neutral or ionized forms, including any salt forms thereof. It is understood by those skilled understand that the specific form will depend on the pH.

As used herein, “6-aminocaproate,” having the chemical formula —OOC—(CH2)5-NH2 (see FIG. 1, and abbreviated as 6-ACA), is the ionized form of 6-aminocaproic acid (IUPAC name 6-aminohexanoic acid), and it is understood that 6-aminocaproate and 6-aminocaproic acid can be used interchangeably throughout to refer to the compound in any of its neutral or ionized forms, including any salt forms thereof. It is understood by those skilled understand that the specific form will depend on the pH.

As used herein, “caprolactam” (IUPAC name azepan-2-one) is a lactam of 6-aminohexanoic acid (see FIG. 1, and abbreviated as CPO).

As used herein, “hexamethylenediamine,” also referred to as 1,6-diaminohexane or 1,6-hexanediamine, has the chemical formula H2N(CH2)6NH2 (see FIG. 1 and abbreviated as HMD).

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

As used herein, the term “osmoprotectant” when used in reference to a culture or growth condition is intended to mean a compound that acts as an osmolyte and helps a microbial organism as described herein survive osmotic stress. Osmoprotectants include, for example, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine.

As used herein, the term “growth-coupled” when used in reference to the production of a biochemical is intended to mean that the biosynthesis of the referenced biochemical is produced during the growth phase of a microorganism. In a particular embodiment, the growth-coupled production can be obligatory, meaning that the biosynthesis of the referenced biochemical is an obligatory product produced during the growth phase of a microorganism.

As used herein, “metabolic modification” is intended to refer to a biochemical reaction that is altered from its naturally occurring state. Metabolic modifications can include, for example, elimination of a biochemical reaction activity by functional disruptions of one or more genes encoding an enzyme participating in the reaction.

As used herein, the term “gene disruption,” or grammatical equivalents thereof, is intended to mean a genetic alteration that renders the encoded gene product inactive. The genetic alteration can be, for example, deletion of the entire gene, deletion of a regulatory sequence required for transcription or translation, deletion of a portion of the gene which results in a truncated gene product, or by any of various mutation strategies that inactivate the encoded gene product. One particularly useful method of gene disruption is complete gene deletion because it reduces or eliminates the occurrence of genetic reversions in the non-naturally occurring microorganisms.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism.

The term “heterologous” refers to a molecule, material, or activity derived from a source other than the referenced species whereas “homologous” refers to a molecule, material, or activity derived from the host microbial organism. Accordingly, exogenous expression of an encoding nucleic acid can utilize either or both a heterologous or homologous encoding nucleic acid.

As used herein the term “about” means±10% of the stated value. The term “about” can mean rounded to the nearest significant digit. Thus, about 5% means 4.5% to 5.5%. Additionally, about in reference to a specific number also includes that exact number. For example, about 5% also includes exact 5%.

A used herein, the term turnover number (also termed as kcat) is defined as the maximum number of chemical conversions of substrate molecules per second that a single catalytic site will execute for a given enzyme concentration [ET]. It can be calculated from the maximum reaction rate Vmax and catalyst site concentration [ET] as follows:

Kcat=V max/[ET]. The unit is s−1.

As used herein the term “catalytic efficiency” is a measure of how efficiently an enzyme converts substrates into products. A comparison of catalytic efficiencies can also be used as a measure of the preference of an enzyme for different substrates (i.e., substrate specificity). The higher the catalytic efficiency, the more the enzyme “prefers” that substrate. It can be calculated from the formula: kcat/KM, where kcat is the turnover number and KM is the Michaelis constant, KM is the substrate concentration at which the reaction rate is half of Vmax. The unit of catalytic efficiency can be expressed as s−1M−1.

As used herein the term “bioderived” in the context of 6-aminocaproic acid, 1,6-hexanediol, caprolactone, caprolactam, or hexamethylenediamine means that these compounds are synthesized in a microbial organism.

It is understood that when more than one exogenous nucleic acid is included in a microbial organism, the exogenous nucleic acids refer to the referenced encoding nucleic acid or biosynthetic activity, as discussed above. It is further understood, as disclosed herein, that such exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, which are not integrated into the host chromosome, and the plasmids remain as extra-chromosomal elements, and still be considered as two or more exogenous nucleic acids. The number of referenced exogenous nucleic acids or biosynthetic activities refers to the number of encoding nucleic acids or the number of biosynthetic activities, not the number of separate nucleic acids introduced into the host organism.

The non-naturally occurring microbial organisms can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

In the case of gene disruptions, a particularly useful stable genetic alteration is a gene deletion. The use of a gene deletion to introduce a stable genetic alteration is particularly useful to reduce the likelihood of a reversion to a phenotype prior to the genetic alteration. For example, stable growth-coupled production of a biochemical can be achieved, for example, by deletion of a gene encoding an enzyme catalyzing one or more reactions within a set of metabolic modifications. The stability of growth-coupled production of a biochemical can be further enhanced through multiple deletions, significantly reducing the likelihood of multiple compensatory reversions occurring for each disrupted activity.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less than 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms having 6-aminocaproic acid, caprolactam, hexamethylenediamine or levulinic acid biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. In gene disruption strategies, evolutionally related genes can also be disrupted or deleted in a host microbial organism, paralogs or orthologs, to reduce or eliminate activities to ensure that any functional redundancy in enzymatic activities targeted for disruption do not short circuit the designed metabolic modifications.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.2.29+(Jan. 14, 2014) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

It is understood that any of the pathways disclosed herein, including those as described in the Figures can be used to generate a non-naturally occurring microbial organism that produces any pathway intermediate or product, as desired. As disclosed herein, such a microbial organism that produces an intermediate can be used in combination with another microbial organism expressing downstream pathway enzymes to produce a desired product. However, it is understood that a non-naturally occurring microbial organism that produces a 6-aminocaproic acid, caprolactam, hexamethylenediamine or levulinic acid pathway intermediate can be utilized to produce the intermediate as a desired product.

Described herein with general reference to the metabolic reaction, reactant or product thereof, or with specific reference to one or more nucleic acids or genes encoding an enzyme associated with or catalyzing the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze the referenced reaction, reactant or product. Likewise, given the well-known fields of metabolic biochemistry, enzymology and genomics, reference herein to a gene or encoding nucleic acid also constitutes a reference to the corresponding encoded enzyme and the reaction it catalyzes as well as the reactants and products of the reaction.

The non-naturally occurring microbial organisms can be produced by introducing expressible nucleic acids encoding one or more of the enzymes participating in one or more 6-aminocaproic acid, caprolactam, hexamethylenediamine or levulinic acid biosynthetic pathways. Depending on the host microbial organism chosen for biosynthesis, nucleic acids for some or all of a particular 6-aminocaproic acid, caprolactam, hexamethylenediamine or levulinic acid biosynthetic pathway can be expressed. For example, if a chosen host is deficient in one or more enzymes for a desired biosynthetic pathway, then expressible nucleic acids for the deficient enzyme(s) are introduced into the host for subsequent exogenous expression. Alternatively, if the chosen host exhibits endogenous expression of some pathway genes, but is deficient in others, then an encoding nucleic acid is needed for the deficient enzyme(s) to achieve 6-aminocaproic acid, caprolactam, hexamethylenediamine or levulinic acid biosynthesis. Thus, a non-naturally occurring microbial organism can be produced by introducing exogenous enzyme activities to obtain a desired biosynthetic pathway or a desired biosynthetic pathway can be obtained by introducing one or more exogenous enzyme activities that, together with one or more endogenous enzymes, produce a desired product such as 6-aminocaproic acid, caprolactam, hexamethylenediamine or levulinic acid.

Depending on the 6-aminocaproic acid, caprolactam, hexamethylenediamine or levulinic acid biosynthetic pathway constituents of a selected host microbial organism, the non-naturally occurring microbial organisms will include at least one exogenously expressed 6-aminocaproic acid, caprolactam, hexamethylenediamine or levulinic acid pathway-encoding nucleic acid and up to all encoding nucleic acids for one or more adipate, 6-aminocaproic acid or caprolactam biosynthetic pathways. For example, 6-aminocaproic acid, caprolactam, hexamethylenediamine or levulinic acid biosynthesis can be established in a host deficient in a pathway enzyme through exogenous expression of the corresponding encoding nucleic acid. In a host deficient in all enzymes of a 6-aminocaproic acid, caprolactam, hexamethylenediamine or levulinic acid pathway, exogenous expression of all enzymes in the pathway can be included, although it is understood that all enzymes of a pathway can be expressed even if the host contains at least one of the pathway enzymes.

Given the teachings and guidance provided herein, those skilled in the art will understand that the number of encoding nucleic acids to introduce in an expressible form will, at least, parallel the adipate, 6-aminocaproic acid, caprolactam, hexamethylenediamine or levulinic acid pathway deficiencies of the selected host microbial organism. Therefore, a non-naturally occurring microbial organism can have at least one, two, three, four, five, six, seven, eight, nine, ten, eleven or twelve, up to all nucleic acids encoding the above enzymes constituting a 6-aminocaproic acid, caprolactam, hexamethylenediamine or levulinic acid biosynthetic pathway. In some embodiments, the non-naturally occurring microbial organisms also can include other genetic modifications that facilitate or optimize 6-aminocaproic acid, caprolactam, hexamethylenediamine or levulinic acid biosynthesis or that confer other useful functions onto the host microbial organism. One such other functionality can include, for example, augmentation of the synthesis of one or more of the 6-aminocaproic acid, caprolactam, hexamethylenediamine or levulinic acid pathway precursors such as succinyl-CoA and/or acetyl-CoA in the case of adipate synthesis, or adipyl-CoA or adipate in the case of 6-aminocaproic acid or caprolactam synthesis, including the adipate pathway enzymes disclosed herein, or pyruvate and succinic semialdhyde, glutamate, glutaryl-CoA, homolysine or 2-amino-7-oxosubarate in the case of 6-aminocaprioate synthesis, or 6-aminocaproate, glutamate, glutaryl-CoA, pyruvate and 4-aminobutanal, or 2-amino-7-oxosubarate in the case of hexamethylenediamine synthesis.

In some embodiments, a non-naturally occurring microbial organism has at least one exogenous nucleic acid encoding a trans-enoyl-CoA reductase that reacts with CPCoA to form adipyl CoA and selected from TERs comprising the amino acid sequences having at least about 50% amino acid sequence identity to at least 25, 50, 75, 100, 150, 200, 250, 300, or more contiguous amino acids of any of any of SEQ ID NOs: 1, 4-9, 11, 14, 15, or 17-26.

Generally, a host microbial organism is selected such that it produces the precursor of a 6-aminocaproic acid, caprolactam, or hexamethylenediamine pathway, either as a naturally produced molecule or as an engineered product that either provides de novo production of a desired precursor or increased production of a precursor naturally produced by the host microbial organism. A host organism can be engineered to increase production of a precursor, as disclosed herein. In addition, a microbial organism that has been engineered to produce a desired precursor can be used as a host organism and further engineered to express enzymes or proteins of a 6-aminocaproic acid, caprolactam, hexamethylenediamine or levulinic acid pathway.

In some embodiments, a non-naturally occurring microbial organism is generated from a host that contains the enzymatic capability to synthesize 6-aminocaproic acid, caprolactam, hexamethylenediamine or levulinic acid. In this specific embodiment it can be useful to increase the synthesis or accumulation of a 6-aminocaproic acid, caprolactam, hexamethylenediamine or levulinic acid pathway product to, for example, drive 6-aminocaproic acid, caprolactam, hexamethylenediamine or levulinic acid pathway reactions toward 6-aminocaproic acid, caprolactam, hexamethylenediamine or levulinic acid production. Increased synthesis or accumulation can be accomplished by, for example, overexpression of nucleic acids encoding one or more of the above-described 6-aminocaproic acid, caprolactam, hexamethylenediamine or levulinic acid pathway enzymes. Over expression of the 6-aminocaproic acid, caprolactam, hexamethylenediamine or levulinic acid pathway enzyme or enzymes can occur, for example, through exogenous expression of the endogenous gene or genes, or through exogenous expression of the heterologous gene or genes. Therefore, naturally occurring organisms can be readily generated to be non-naturally occurring microbial organisms, for example, producing 6-aminocaproic acid, caprolactam, hexamethylenediamine or levulinic acid, through overexpression of at least one, two, three, four, five, six, seven, eight, nine, ten, eleven, twelve, thirteen, fourteen, that is, up to all nucleic acids encoding 6-aminocaproic acid, caprolactam, hexamethylenediamine or levulinic acid biosynthetic pathway enzymes. In addition, a non-naturally occurring organism can be generated by mutagenesis of an endogenous gene that results in an increase in activity of an enzyme in the 6-aminocaproic acid, caprolactam, hexamethylenediamine or levulinic acid biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

In some embodiments, a non-naturally occurring microbial organism includes one or more gene disruptions, where the organism produces a 6-ACA, adipate and/or HMDA. The disruptions occur in genes encoding an enzyme that couples production of adipate, 6-ACA and/or HMDA to growth of the organism when the gene disruption reduces the activity of the enzyme, such that the gene disruptions confer increased production of adipate, 6-ACA and/or HMDA onto the non-naturally occurring organism. Thus, in some embodiments is provided a non-naturally occurring microbial organism, comprising one or more gene disruptions, the one or more gene disruptions occurring in genes encoding proteins or enzymes wherein the one or more gene disruptions confer increased production of adipate, 6-ACA and/or HMDA in the organism. As disclosed herein, such an organism contains a pathway for production of adipate, 6-ACA and/or HMDA.

It is understood that, in methods, any of the one or more exogenous nucleic acids can be introduced into a microbial organism to produce a non-naturally occurring microbial organism. The nucleic acids can be introduced so as to confer, for example, a 6-aminocaproic acid, caprolactam, hexamethylenediamine or levulinic acid biosynthetic pathway onto the microbial organism. Alternatively, encoding nucleic acids can be introduced to produce an intermediate microbial organism having the biosynthetic capability to catalyze some of the required reactions to confer 6-aminocaproic acid, caprolactam, hexamethylenediamine or levulinic acid biosynthetic capability. For example, a non-naturally occurring microbial organism having a 6-aminocaproic acid, caprolactam, hexamethylenediamine or levulinic acid biosynthetic pathway can comprise at least two exogenous nucleic acids encoding desired enzymes. In the case of adipate production, at least two exogenous nucleic acids can encode the enzymes such as the combination of succinyl-CoA: acetyl-CoA acyl transferase and 3-hydroxyacyl-CoA dehydrogenase, or succinyl-CoA: acetyl-CoA acyl transferase and 3-hydroxyadipyl-CoA dehydratase, or 3-hydroxyadipyl-CoA and 5-carboxy-2-pentenoyl-CoA reductase, or 3-hydroxyacyl-CoA and adipyl-CoA synthetase, and the like. In the case of caprolactam production, at least two exogenous nucleic acids can encode the enzymes such as the combination of CoA-dependent trans-enoyl-CoA reductase and transaminase, or CoA-dependent trans-enoyl-CoA reductase and amidohydrolase, or transaminase and amidohydrolase. In the case of 6-aminocaproic acid production, at least two exogenous nucleic acids can encode the enzymes such as the combination of an 4-hydroxy-2-oxoheptane-1,7-dioate (HODH) aldolase and a 2-oxohept-4-ene-1,7-dioate (OHED) hydratase, or a 2-oxohept-4-ene-1,7-dioate (OHED) hydratase and a 2-aminoheptane-1,7-dioate (2-AHD) decarboxylase, a 3-hydroxyadipyl-CoA dehydratase and a adipyl-CoA dehydrogenase, a glutamyl-CoA transferase and a 6-aminopimeloyl-CoA hydrolase, or a glutaryl-CoA beta-ketothiolase and a 3-aminopimelate 2,3-aminomutase. In the case of hexamethylenediamine production, at least two exogenous nucleic acids can encode the enzymes such as the combination of 6-aminocaproate kinase and [(6-aminohexanoyl)oxy]phosphonate (6-AHOP) oxidoreductase, or a 6-acetamidohexanoate kinase and an [(6-acetamidohexanoyl)oxy]phosphonate (6-AAHOP) oxidoreductase, 6-aminocaproate N-acetyltransferase and 6-acetamidohexanoyl-CoA oxidoreductase, a 3-hydroxy-6-aminopimeloyl-CoA dehydratase and a 2-amino-7-oxoheptanoate aminotransferase, or a 3-oxopimeloyl-CoA ligase and a homolysine decarboxylase. Thus, it is understood that any combination of two or more enzymes of a biosynthetic pathway can be included in a non-naturally occurring microbial organism.

Similarly, it is understood that any combination of three or more enzymes of a biosynthetic pathway can be included in a non-naturally occurring microbial organism, for example, in the case of adipate production, the combination of enzymes succinyl-CoA: acetyl-CoA acyl transferase, 3-hydroxyacyl-CoA dehydrogenase, and 3-hydroxyadipyl-CoA dehydratase; or succinyl-CoA: acetyl-CoA acyl transferase, 3-hydroxyacyl-CoA dehydrogenase and 5-carboxy-2-pentenoyl-CoA reductase; or succinyl-CoA: acetyl-CoA acyl transferase, 3-hydroxyacyl-CoA dehydrogenase and adipyl-CoA synthetase; or 3-hydroxyacyl-CoA dehydrogenase, 3-hydroxyadipyl-CoA dehydratase and adipyl-CoA: acetyl-CoA transferase, and so forth, as desired, so long as the combination of enzymes of the desired biosynthetic pathway results in production of the corresponding desired product. In the case of 6-aminocaproic acid production, the at least three exogenous nucleic acids can encode the enzymes such as the combination of an 4-hydroxy-2-oxoheptane-1,7-dioate (HODH) aldolase, a 2-oxohept-4-ene-1,7-dioate (OHED) hydratase and a 2-oxoheptane-1,7-dioate (2-OHD) decarboxylase, or a 2-oxohept-4-ene-1,7-dioate (OHED) hydratase, a 2-aminohept-4-ene-1,7-dioate (2-AHE) reductase and a 2-aminoheptane-1,7-dioate (2-AHD) decarboxylase, or a 3-hydroxyadipyl-CoA dehydratase, 2,3-dehydroadipyl-CoA reductase and a adipyl-CoA dehydrogenase, or a 6-amino-7-carboxyhept-2-enoyl-CoA reductase, a 6-aminopimeloyl-CoA hydrolase and a 2-aminopimelate decarboxylase, or a glutaryl-CoA beta-ketothiolase, a 3-aminating oxidoreductase and a 2-aminopimelate decarboxylase, or a 3-oxoadipyl-CoA thiolase, a 5-carboxy-2-pentenoate reductase and a adipate reductase. In the case of hexamethylenediamine production, at least three exogenous nucleic acids can encode the enzymes such as the combination of 6-aminocaproate kinase, [(6-aminohexanoyl)oxy]phosphonate (6-AHOP) oxidoreductase and 6-aminocaproic semialdhyde aminotransferase, or a 6-aminocaproate N-acetyltransferase, a 6-acetamidohexanoate kinase and an [(6-acetamidohexanoyl)oxy]phosphonate (6-AAHOP) oxidoreductase, or 6-aminocaproate N-acetyltransferase, a [(6-acetamidohexanoyl)oxy]phosphonate (6-AAHOP) acyltransferase and 6-acetamidohexanoyl-CoA oxidoreductase, or a 3-oxo-6-aminopimeloyl-CoA oxidoreductase, a 3-hydroxy-6-aminopimeloyl-CoA dehydratase and a homolysine decarboxylase, or a 2-oxo-4-hydroxy-7-aminoheptanoate aldolase, a 2-oxo-7-aminohept-3-enoate reductase and a homolysine decarboxylase, or a 6-acetamidohexanoate reductase, a 6-acetamidohexanal aminotransferase and a 6-acetamidohexanamine N-acetyltransferase. Similarly, any combination of four or more enzymes of a biosynthetic pathway as disclosed herein can be included in a non-naturally occurring microbial organism, as desired, so long as the combination of enzymes of the desired biosynthetic pathway results in production of the corresponding desired product.

In addition to the biosynthesis of 6-aminocaproic acid, caprolactam, hexamethylenediamine or levulinic acid as described herein, the non-naturally occurring microbial organisms and methods also can be utilized in various combinations with each other and with other microbial organisms and methods well known in the art to achieve product biosynthesis by other routes. For example, one alternative to produce 6-aminocaproic acid, caprolactam, hexamethylenediamine or levulinic acid other than use of the 6-aminocaproic acid, caprolactam, hexamethylenediamine or levulinic acid producers is through addition of another microbial organism capable of converting an adipate, 6-aminocaproic acid or caprolactam pathway intermediate to 6-aminocaproic acid, caprolactam, hexamethylenediamine or levulinic acid. One such procedure includes, for example, the fermentation of a microbial organism that produces a 6-aminocaproic acid, caprolactam, hexamethylenediamine or levulinic acid pathway intermediate. The 6-aminocaproic acid, caprolactam, hexamethylenediamine or levulinic acid pathway intermediate can then be used as a substrate for a second microbial organism that converts the 6-aminocaproic acid, caprolactam, hexamethylenediamine or levulinic acid pathway intermediate to 6-aminocaproic acid, caprolactam, hexamethylenediamine or levulinic acid. The 6-aminocaproic acid, caprolactam, hexamethylenediamine or levulinic acid pathway intermediate can be added directly to another culture of the second organism or the original culture of 6-aminocaproic acid, caprolactam, hexamethylenediamine or levulinic acid pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods can be assembled in a wide variety of sub pathways to achieve biosynthesis of, for example, 6-aminocaproic acid, caprolactam, hexamethylenediamine or levulinic acid. In these embodiments, biosynthetic pathways for a desired product can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of 6-aminocaproic acid, caprolactam, hexamethylenediamine or levulinic acid can be accomplished by constructing a microbial organism that contains biosynthetic pathways for conversion of one pathway intermediate to another pathway intermediate or the product. Alternatively, 6-aminocaproic acid, caprolactam, hexamethylenediamine or levulinic acid also can be biosynthetically produced from microbial organisms through co-culture or co-fermentation using two organisms in the same vessel, where the first microbial organism produces a 6-aminocaproic acid, caprolactam, hexamethylenediamine or levulinic acid intermediate and the second microbial organism converts the intermediate to 6-aminocaproic acid, caprolactam, hexamethylenediamine or levulinic acid.

Given the teachings and guidance provided herein, those skilled in the art will understand that a wide variety of combinations and permutations exist for the non-naturally occurring microbial organisms and methods together with other microbial organisms, with the co-culture of other non-naturally occurring microbial organisms having sub pathways and with combinations of other chemical and/or biochemical procedures well known in the art to produce 6-aminocaproic acid, caprolactam, hexamethylenediamine or levulinic acid.

Similarly, it is understood by those skilled in the art that a host organism can be selected based on desired characteristics for introduction of one or more gene disruptions to increase production of 6-aminocaproic acid, caprolactam, hexamethylenediamine or levulinic acid. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods can be applied to any suitable host microorganism to identify the cognate metabolic alterations needed to construct an organism in a species of interest that will increase 6-aminocaproic acid, caprolactam, hexamethylenediamine or levulinic acid biosynthesis. In a particular embodiment, the increased production couples biosynthesis of 6-aminocaproic acid, caprolactam, hexamethylenediamine or levulinic acid to growth of the organism, and can obligatorily couple production of 6-aminocaproic acid, caprolactam, hexamethylenediamine or levulinic acid to growth of the organism if desired and as disclosed herein.

Sources of encoding nucleic acids for a 6-aminocaproic acid, caprolactam, hexamethylenediamine or levulinic acid pathway enzyme can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. In some embodiments, the source of the encoding nucleic acids for a 6-aminocaproic acid, caprolactam, hexamethylenediamine or levulinic acid pathway enzyme is shown in Table 3. In some embodiments, the source of the encoding nucleic acids for trans-enoyl-CoA reductase enzyme is shown in Table 3. In other embodiments, the source of the encoding nucleic acids for trans-enoyl-CoA reductase enzyme is Candida, Drosophila, Arabidopsis, or Homo sapiens. In some embodiments, the source of the encoding nucleic acids for a 6-aminocaproic acid, caprolactam, hexamethylenediamine or levulinic acid pathway enzyme are species such as, Escherichia coli, Escherichia coli str. K12, Escherichia coli C, Escherichia coli Crooks (ATCC 8739), Escherichia coli W, Pseudomonas sp, Pseudomonas knackmussii, Pseudomonas sp. Strain B13, Pseudomonas putida, Pseudomonas fluorescens, Pseudomonas stutzeri, Pseudomonas mendocina, Rhodopseudomonas palustris, Mycobacterium tuberculosis, Vibrio cholera, Heliobacter pylori, Klebsiella pneumoniae, Serratia proteamaculans, Streptomyces sp. 2065, Pseudomonas aeruginosa, Pseudomonas aeruginosa PAO1, Ralstonia eutropha, Ralstonia eutropha H16, Clostridium acetobutylicum, Euglena gracilis, Treponema denticola, Clostridium kluyveri, Homo sapiens, Rattus norvegicus, Acinetobacter sp. ADP1, Acinetobacter sp. Strain M−1, Streptomyces coelicolor, Eubacterium barkeri, Peptostreptococcus asaccharolyticus, Clostridium botulinum, Clostridium botulinum A3 str, Clostridium tyrobutyricum, Clostridium pasteurianum, Clostridium thermoaceticum (Moorella thermoaceticum), Moorella thermoacetica Acinetobacter calcoaceticus, Mus musculus, Sus scrofa, Flavobacterium sp, Arthrobacter aurescens, Penicillium chrysogenum, Aspergillus niger, Aspergillus nidulans, Bacillus subtilis, Saccharomyces cerevisiae, Zymomonas mobilis, Mannheimia succiniciproducens, Clostridium ljungdahlii, Clostridium carboxydivorans, Geobacillus stearothermophilus, Agrobacterium tumefaciens, Achromobacter denitrificans, Arabidopsis thaliana, Haemophilus influenzae, Acidaminococcus fermentans, Clostridium sp. M62/1, Fusobacterium nucleatum, Bos taurus, Zoogloea ramigera, Rhodobacter sphaeroides, Clostridium beijerinckii, Metallosphaera sedula, Thermoanaerobacter species, Thermoanaerobacter brockii, Acinetobacter baylyi, Porphyromonas gingivalis, Leuconostoc mesenteroides, Sulfolobus tokodaii, Sulfolobus tokodaii 7, Sulfolobus solfataricus, Sulfolobus solfataricus, Sulfolobus acidocTERarius, Salmonella typhimurium, Salmonella enterica, Thermotoga maritima, Halobacterium salinarum, Bacillus cereus, Clostridium difficile, Alkaliphilus metalliredigenes, Thermoanaerobacter tengcongensis, Saccharomyces kluyveri, Helicobacter pylori, Corynebacterium glutamicum, Clostridium saccharoperbutylacetonicum, Pseudomonas chlororaphis, Streptomyces clavuligerus, Campylobacter jejuni, Thermus thermophilus, Pelotomaculum thermopropionicum, Bacteroides capillosus, Anaerotruncus colihominis, Natranaerobius thermophilius, Archaeoglobus fulgidus, Archaeoglobus fulgidus DSM 4304, Haloarcula marismortui, Pyrobaculum aerophilum, Pyrobaculum aerophilum str. IM2, Nicotiana tabacum, Menthe piperita, Pinus taeda, Hordeum vulgare, Zea mays, Rhodococcus opacus, Cupriavidus necator, Bradyrhizobium japonicum, Bradyrhizobium japonicum USDA110, Ascarius suum, butyrate-producing bacterium L2-50, Bacillus megaterium, Methanococcus maripaludis, Methanosarcina mazei, Methanosarcina mazei, Methanocarcina barkeri, MethanocTERococcus jannaschii, Caenorhabditis elegans, Leishmania major, Methylomicrobium alcaliphilum 20Z, Chromohalobacter salexigens, Archaeglubus fulgidus, Chlamydomonas reinhardtii, Trichomonas vaginalis G3, Trypanosoma brucei, Mycoplana ramose, Micrococcus luteas, Acetobacter pasteurians, Kluyveromyces lactis, Mesorhizobium loti, Lactococcus lactis, Lysinibacillus sphaericus, Candida boidinii, Candida albicans SC5314, Burkholderia ambifaria AMMD, Ascaris suun, Acinetobacter baumanii, Acinetobacter calcoaceticus, Burkholderia phymatum, Candida albicans, Clostridium subterminale, Cupriavidus taiwanensis, Flavobacterium lutescens, Lachancea kluyveri, Lactobacillus sp. 30a, Leptospira interrogans, Moorella thermoacetica, Myxococcus xanthus, Nicotiana glutinosa, Nocardia iowensis (sp. NRRL 5646), Pseudomonas reinekei MT1, Ralstonia eutropha JMP134, Ralstonia metallidurans, Rhodococcus jostii, Schizosaccharomyces pombe, Selenomonas ruminantium, Streptomyces clavuligenus, Syntrophus aciditrophicus, Vibrio parahaemolyticus, Vibrio vulnificus, as well as other exemplary species disclosed herein or available as source organisms for corresponding genes (see Examples). However, with the complete genome sequence available for now more than 550 species (with more than half of these available on public databases such as the NCBI), including 395 microorganism genomes and a variety of yeast, fungi, plant, and mammalian genomes, the identification of genes encoding the requisite 6-aminocaproic acid, caprolactam, hexamethylenediamine or levulinic acid biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations enabling biosynthesis of 6-aminocaproic acid, caprolactam, hexamethylenediamine or levulinic acid described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when a 6-aminocaproic acid, caprolactam, hexamethylenediamine or levulinic acid biosynthetic pathway exists in an unrelated species, 6-aminocaproic acid, caprolactam, hexamethylenediamine or levulinic acid biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods can be applied to all microbial organisms using the cognate metabolic alterations to those exemplified herein to construct a microbial organism in a species of interest that will synthesize 6-aminocaproic acid, caprolactam, hexamethylenediamine or levulinic acid.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and the like. For example, E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Methods for constructing and testing the expression levels of a non-naturally occurring 6-aminocaproic acid, caprolactam, hexamethylenediamine or levulinic acid-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

Exogenous nucleic acid sequences involved in a pathway for production of 6-aminocaproic acid, caprolactam, hexamethylenediamine or levulinic acid can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more 6-aminocaproic acid, caprolactam, hexamethylenediamine or levulinic acid biosynthetic pathway encoding nucleic acids as exemplified herein operably linked to expression control sequences functional in the host organism. Expression vectors applicable for use in the microbial host organisms include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments are methods for producing a desired intermediate or product such as adipate, 6-aminocaproic acid, caprolactam, hexamethylenediamine or levulinic acid. For example, a method for producing adipate can involve culturing a non-naturally occurring microbial organism having an adipate pathway, the pathway including at least one exogenous nucleic acid encoding an adipate pathway enzyme expressed in a sufficient amount to produce adipate, under conditions and for a sufficient period of time to produce adipate, the adipate pathway including succinyl-CoA: acetyl-CoA acyl transferase, 3-hydroxyacyl-CoA dehydrogenase, 3-hydroxyadipyl-CoA dehydratase, 5-carboxy-2-pentenoyl-CoA reductase, and adipyl-CoA synthetase or phosphotransadipylase/adipate kinase or adipyl-CoA: acetyl-CoA transferase or adipyl-CoA hydrolase. Additionally, a method for producing adipate can involve culturing a non-naturally occurring microbial organism having an adipate pathway, the pathway including at least one exogenous nucleic acid encoding an adipate pathway enzyme expressed in a sufficient amount to produce adipate, under conditions and for a sufficient period of time to produce adipate, the adipate pathway including succinyl-CoA: acetyl-CoA acyl transferase, 3-oxoadipyl-CoA transferase, 3-oxoadipate reductase, 3-hydroxyadipate dehydratase, and 2-enoate reductase.

Further, a method for producing 6-aminocaproic acid can involve culturing a non-naturally occurring microbial organism having a 6-aminocaproic acid pathway, the pathway including at least one exogenous nucleic acid encoding a 6-aminocaproic acid pathway enzyme expressed in a sufficient amount to produce 6-aminocaproic acid, under conditions and for a sufficient period of time to produce 6-aminocaproic acid, the 6-aminocaproic acid pathway including CoA-dependent trans-enoyl-CoA reductase and transaminase or 6-aminocaproate dehydrogenase. Additionally, a method for producing caprolactam can involve culturing a non-naturally occurring microbial organism having a caprolactam pathway, the pathway including at least one exogenous nucleic acid encoding a caprolactam pathway enzyme expressed in a sufficient amount to produce caprolactam, under conditions and for a sufficient period of time to produce caprolactam, the caprolactam pathway including CoA-dependent aldehyde dehydrogenase, transaminase or 6-aminocaproate dehydrogenase, and amidohydrolase.

Suitable purification and/or assays to test for the production of 6-aminocaproic acid, caprolactam, hexamethylenediamine or levulinic acid can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme activities from the exogenous DNA sequences can also be assayed using methods well known in the art.

The 6-aminocaproic acid, caprolactam, hexamethylenediamine or levulinic acid can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products. For example, the 6-aminocaproic acid, caprolactam, hexamethylenediamine or levulinic acid producers can be cultured for the biosynthetic production of 6-aminocaproic acid, caprolactam, hexamethylenediamine or levulinic acid.

For the production of 6-aminocaproic acid, caprolactam, hexamethylenediamine or levulinic acid, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in U.S. Pat. No. 7,947,483 issued May 24, 2011. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microorganism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the methods include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms for the production of 6-aminocaproic acid, caprolactam, hexamethylenediamine or levulinic acid.

In addition to renewable feedstocks such as those exemplified above, the 6-aminocaproic acid, caprolactam, hexamethylenediamine, or levulinic acid microbial organisms also can be modified for growth on syngas as its source of carbon. In this specific embodiment, one or more proteins or enzymes are expressed in the 6-aminocaproic acid, caprolactam, hexamethylenediamine or levulinic acid producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H2 and CO and can be obtained from the gasification of any organic feedstock, including but not limited to coal, coal oil, natural gas, biomass, and waste organic matter. Gasification is generally carried out under a high fuel to oxygen ratio. Although largely H2 and CO, syngas can also include C02 and other gases in smaller quantities. Thus, synthesis gas provides a cost effective source of gaseous carbon such as CO and additionally, C02.

The Wood-Ljungdahl pathway catalyzes the conversion of CO and H2 to acetyl-CoA and other products such as acetate. Organisms capable ofutilizing CO and syngas also generally have the capability of utilizing C02 and C02/H2 mixtures through the same basic set of enzymes and transformations encompassed by the Wood-Ljungdahl pathway. H2-dependent conversion of C02 to acetate by microorganisms was recognized long before it was revealed that CO also could be used by the same organisms and that the same pathways were involved. Many acetogens have been shown to grow in the presence of C02 and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO2+4H2+nADP+nPi→CH3COOH+2H2O+nATP

Hence, non-naturally occurring microorganisms possessing the Wood-Ljungdahl pathway can utilize CO2 and H2 mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-TiF) whereas the carbonyl branch converts methyl-THF to acetyl-CoA. The reactions in the methyl branch are catalyzed in order by the following enzymes: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: cobalamide corrinoid/iron-sulfur protein, methyltransferase, carbon monoxide dehydrogenase, acetyl-CoA synthase, acetyl-CoA synthase disulfide reductase and hydrogenase, and these enzymes can also be referred to as methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a 6-aminocaproic acid, caprolactam, hexamethylenediamine or levulinic acid pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms such that the modified organism contains the complete Wood-Ljungdahl pathway will confer syngas utilization ability.

Additionally, the reductive (reverse) tricarboxylic acid cycle coupled with carbon monoxide dehydrogenase and/or hydrogenase activities can also be used for the conversion of CO, CO2 and/or H2 to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate: ferredoxin oxidoreductase, succinyl-CoA synthetase, succinyl-CoA transferase, fumarate reductase, fumarase, malate dehydrogenase, NAD(P)Ferredoxin oxidoreductase, carbon monoxide dehydrogenase, and hydrogenase. Specifically, the reducing equivalents extracted from CO and/or H2 by carbon monoxide dehydrogenase and hydrogenase are utilized to fix CO2 via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to the p-toluate, terepathalate, or (2-hydroxy-3-methyl-4-oxobutoxy) phosphonate precursors, glycerAldehyde-3-phosphate, phosphoenolpyruvate, and pyruvate, by pyruvate: ferredoxin oxidoreductase and the enzymes of gluconeogenesis. Following the teachings and guidance provided herein for introducing a sufficient number of encoding nucleic acids to generate a p-toluate, terephthalate or (2-hydroxy-3-methyl-4-oxobutoxy) phosphonate pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms such that the modified organism contains the complete reductive TCA pathway will confer syngas utilization ability.

Given the teachings and guidance provided herein, those skilled in the art will understand that a non-naturally occurring microbial organism can be produced that secretes the biosynthesized compounds when grown on a carbon source such as a carbohydrate. Such compounds include, for example, 6-aminocaproic acid, caprolactam, hexamethylenediamine or levulinic acid and any of the intermediate metabolites in the 6-aminocaproic acid, caprolactam, hexamethylenediamine or levulinic acid pathway. All that is required is to engineer in one or more of the required enzyme activities to achieve biosynthesis of the desired compound or intermediate including, for example, inclusion of some or all of the 6-aminocaproic acid, caprolactam, hexamethylenediamine or levulinic acid biosynthetic pathways. Accordingly, some embodiments provide a non-naturally occurring microbial organism that produces and/or secretes 6-aminocaproic acid, caprolactam, hexamethylenediamine or levulinic acid when grown on a carbohydrate and produces and/or secretes any of the intermediate metabolites shown in the 6-aminocaproic acid, caprolactam, hexamethylenediamine or levulinic acid pathway when grown on a carbohydrate. For example, an adipate producing microbial organisms can initiate synthesis from an intermediate, for example, 3-oxoadipyl-CoA, 3-hydroxyadipyl-CoA, 5-carboxy-2-pentenoyl-CoA, or adipyl-CoA (see FIG. 1), as desired. In addition, an adipate producing microbial organism can initiate synthesis from an intermediate, for example, 3-oxoadipyl-CoA, 3-oxoadipate, 3-hydroxyadipate, or hexa-2-enedioate. The 6-aminocaproic acid producing microbial organism can initiate synthesis from an intermediate, for example, adipate semialdhyde. The caprolactam producing microbial organism can initiate synthesis from an intermediate, for example, adipate semialdhyde or 6-aminocaproic acid (see FIG. 1), as desired.

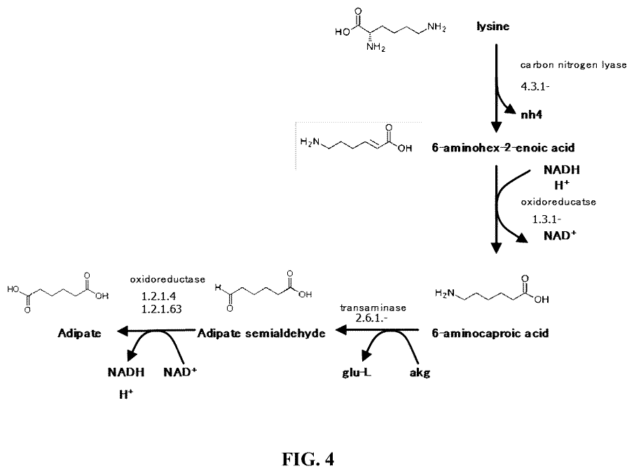

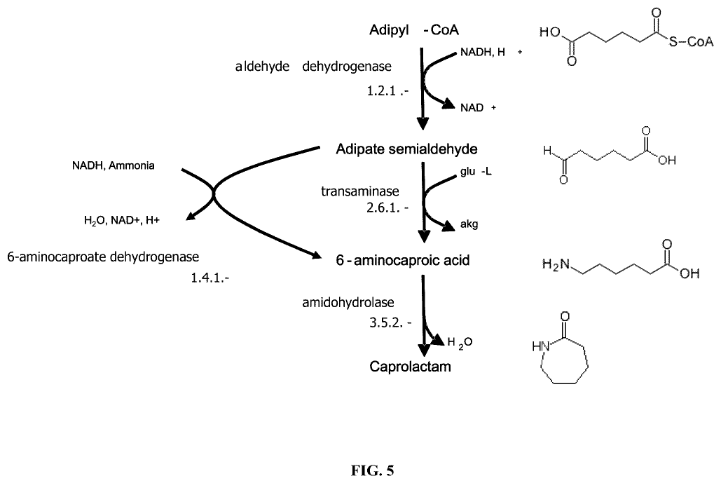

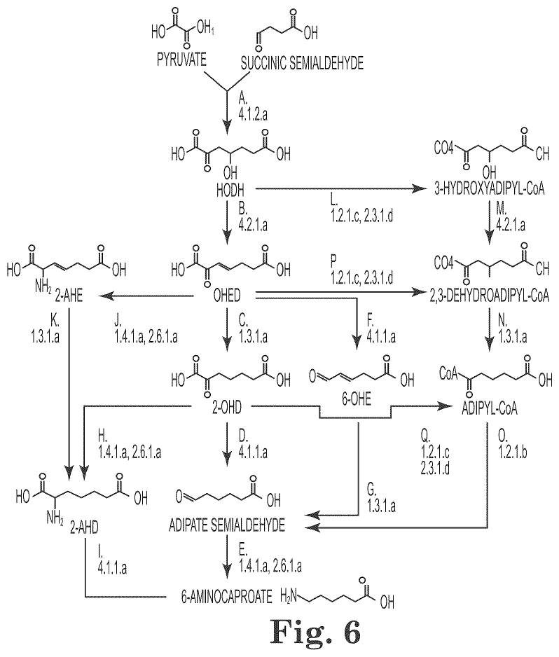

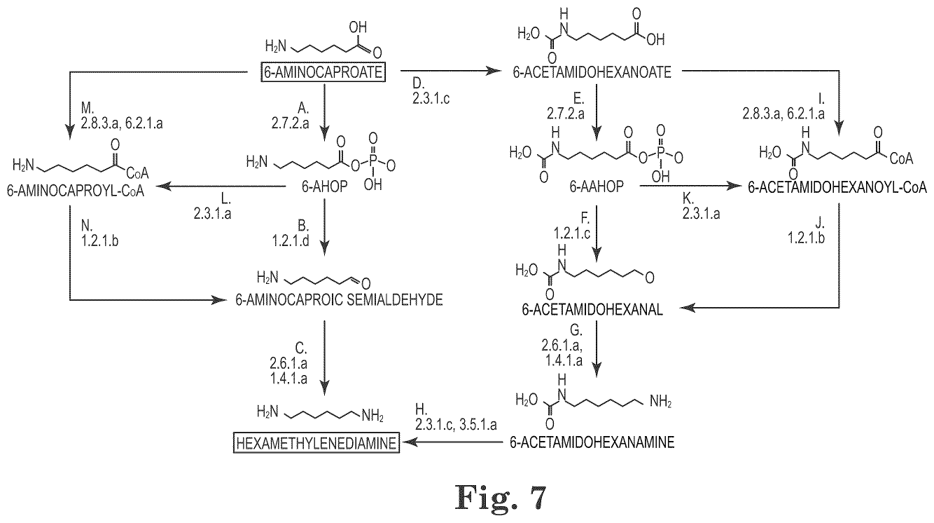

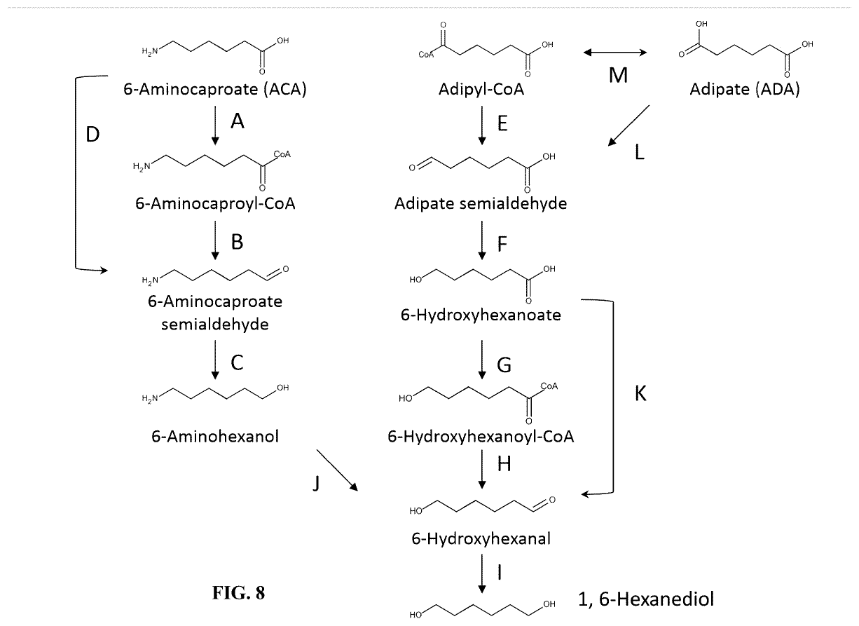

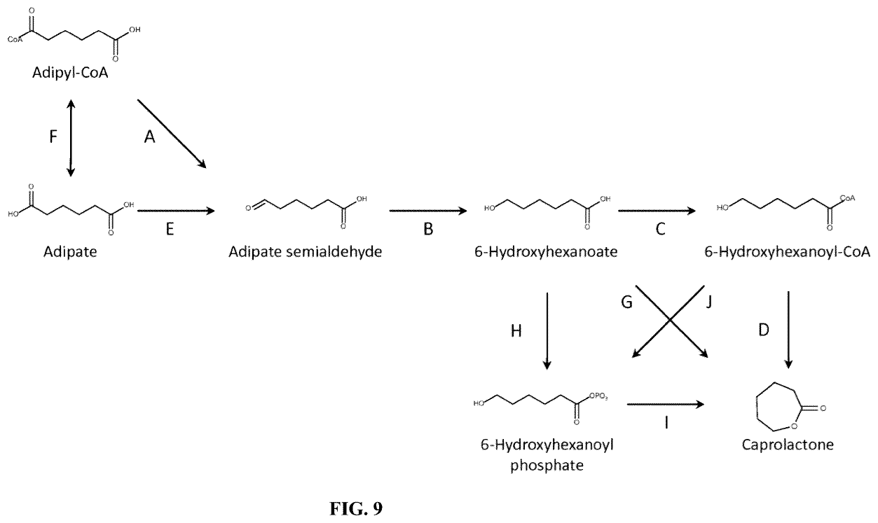

In some embodiments, the non-naturally occurring microorganisms can generate adipate, 6ACA, hexamethylenediamine, caprolatone, or caproclactam as shown in FIG. 4-9.

In some embodiments, the non-naturally occurring microbial organisms further include an exogenously expressed nucleic acid encoding an aldehyde dehydrogenase (ALD) or a transaminase (TA) or both. The ALD reacts with adipyl-CoA to produce adipate semialdehyde, whereas the TA reacts with adipate semialdehyde to produce 6-aminocaproic acid.

In some embodiments, the ALD enzymes have greater catalytic efficiency and activity for the adipyl CoA substrate as compared to succinyl-CoA, or acetyl-CoA, or both substrates. In some embodiments, the ALD enzymes are as shown in Table 1.

TABLE 1
Activity of Aldehyde Dehydrogenases on Adipyl-CoA

| | | Accession No/ | Activity - | Activity - |
|---|---|---|---|---|
| NO. | Organism | SEQ ID NO. | NADH | NADPH |
| 1 | Clostridium kluyveri DSM555 | SEQ ID NO: 27 | − | + |
| 2 | Porphyromonas gingivalis W83 | SEQ ID NO: 28 | + | − |
| 3 | Clostridium difficile 630 | SEQ ID NO: 29 | − | + |
| 4 | Kluyvera intestini | WP_071196317.1 | + | − |
| 5 | Clostridium neonatale | WP_058295546.1 | − | − |
| 6 | Aerococcus sp. HMSC062B07 | WP_070558456.1 | − | − |
| 7 | Peptostreptococcaceae bacterium | WP_021676458.1 | + | − |
| | oral | | | |
| 8 | Dasania marina | WP_026244399.1 | − | − |
| 9 | Porphyromonadaceae bacterium | WP_036830068.1 | − | − |
| | COT-184 | | | |
| 10 | Clostridium lundense | WP_027623222.1 | − | − |
| 11 | Anaerocolumna jejuensis | WP_073279774.1 | + | − |
| 12 | Clostridium homopropionicum | WP_052222510.1 | − | − |
| 13 | Geosporobacter ferrireducens | WP_069981616.1 | − | − |
| 14 | Listeria ivanovii | WP_038407128.1 | − | − |
| 15 | Bacillus soli | WP_066062455.1 | + | − |
| 16 | Enterococcus rivorum | WP_069697141.1 | − | − |
| 17 | Desnuesiella massiliensis | WP_055665162.1 | + | − |
| 18 | Bacteroidales bacterium KA00251 | WP_066041885.1 | − | − |
| 19 | Caldanaerobius | WP_026487268.1 | + | − |
| | polysaccharolyticus | | | |
| 20 | Clostridium sp. ASF356 | WP_004036483.1 | − | − |
| 21 | Clostridiales bacterium DRI-13 | WP_034420506.1 | − | − |
| 22 | Fusobacterium ulcerans ATCC | WP_005981617.1 | − | − |
| | 49185 | | | |
| 23 | Anaerocolumna jejuensis | WP_073279351.1 | − | − |
| 24 | Cellulosilyticum sp. I15G10I2 | WP_070001026.1 | + | − |
| 25 | Geosporobacter ferrireducens | WP_083273866.1 | + | − |
| 26 | Pelosinus sp. UFO1 | WP_038668911.1 | − | − |
| 27 | Bacillus korlensis | WP_084362095.1 | + | − |
| 28 | Acidaminococcus massiliensis | WP_075579339.1 | + | − |
| 29 | Eubacterium sp. SB2 | WP_050640767.1 | − | − |
| 30 | Erwinia teleogrylli | WP_058911295.1 | − | − |
| 31 | Lachnospiraceae bacterium 32 | WP_016223553.1 | + | − |
| 32 | Eubacterium plexicaudatum | WP_004061597.1 | + | − |
| 33 | Clostridium sp. KNHs205 | WP_033166114.1 | + | − |
| 34 | Butyricimonas virosa | WP_027200274.1 | − | − |
| 35 | Malonomonas rubra | WP_072908980.1 | − | − |
| 36 | Robinsoniella peoriensis | WP_044292972.1 | + | − |
| 37 | Clostridium taeniosporum | WP_069679818.1 | − | − |
| 38 | Caldithrix abyssi | WP_006928331.1 | + | − |
| 39 | Piscicoccus intestinalis | WP_084343789.1 | − | − |
| 40 | Sporomusa sphaeroides | WP_075753933.1 | + | − |
| 41 | Bacillus sp. FJAT-25547 | WP_057762439.1 | + | − |
| 42 | Dorea sp. D27 | WP_049729435.1 | + | − |
| 43 | Oscillibacter sp. 13 | WP_081646270.1 | − | − |
| 44 | Enterococcus phoeniculicola | WP_010767571.1 | + | − |
| 45 | Blautia schinkii | WP_044941637.1 | + | − |
| 46 | Shuttleworthia satelles DSM 14600 | WP_006905683.1 | − | − |
| 47 | Clostridium intestinale | WP_073018444.1 | + | − |
| 48 | Massilioclostridium coli | WP_069989048.1 | − | − |
| 49 | Cloacibacillus porcorum | WP_066745012.1 | − | − |
| 50 | Clostridium sp. CL-2 | WP_032120205.1 | − | − |
| 51 | Clostridia bacterium UC5.1-1D10 | WP_054330586.1 | − | − |
| 52 | Methylobacterium sp. CCH5-D2 | WP_082772960.1 | − | − |
| 53 | Sporosarcina globispora | WP_053435653.1 | + | + |
| 54 | Lachnospiraceae bacterium | WP_031546337.1 | − | − |
| | AC3007 | | | |
| 55 | Lachnospiraceae bacterium 28-4 | WP_016290199.1 | − | − |
| 56 | Enterococcus avium | WP_034875865.1 | − | − |
| 57 | Desulfotomaculum | WP_027356260.1 | − | − |
| | thermocisternum | | | |
| 58 | Rhodobacter aestuarii | WP_076486054.1 | + | − |
| 59 | Clostridium grantii | WP_073337420.1 | + | − |
| 60 | Collinsella sp. GD7 | WP_066830323.1 | + | − |
| 61 | Clostridium estertheticum | WP_071611886.1 | − | − |
| 62 | bacterium MS4 | WP_038325413.1 | − | − |
| 63 | Clostridium glycyrrhizinilyticum | WP_009268007.1 | + | − |
| 64 | Bacillus horikoshii | WP_082892049.1 | − | − |
| 65 | Thermincola ferriacetica | WP_052218568.1 | + | − |
| 66 | Lachnospiraceae bacterium | WP_035653923.1 | + | − |
| | AC3007 | | | |
| 67 | Eubacterium sp. 14-2 | WP_016216571.1 | + | − |
| 68 | Candidates Marispirochaeta | WP_069895590.1 | − | − |
| | associata | | | |
| 69 | Clostridium drakei | WP_032078293.1 | − | − |
| 70 | Halanaerobium kushneri | WP_076543773.1 | − | − |
| 71 | Clostridium fallax | WP_072896506.1 | − | − |
| 72 | Flavonifractor plautii | WP_009261118.1 | − | − |
| 73 | Clostridium propionicum | WP_066049640.1 | − | − |
| 74 | Anaerosalibacter massiliensis | WP_042682918.1 | + | − |
| 75 | Clostridium indolis DSM 755 | WP_024295710.1 | + | − |
| 76 | Gabonibacter massiliensis | WP_059027034.1 | − | − |
| 77 | Catabacter hongkongensis | WP_046444791.1 | + | + |
| 78 | Desulfitibacter alkalitolerans | WP_028307735.1 | − | − |
| 79 | Porphyromonas levii | WP_018357742.1 | − | − |
| 80 | Bacillus thermotolerans | WP_039235348.1 | + | − |
| 81 | Desulfitibacter alkalitolerans | WP_028307055.1 | − | − |
| 82 | Gracilibacillus kekensis | WP_073203236.1 | + | + |
| 83 | Lactonifactor longoviformis | WP_072848455.1 | − | − |
| 84 | Propionispora sp. 2/2-37 | WP_054258533.1 | + | − |
| 85 | Erysipelothrix larvae | WP_067632640.1 | − | − |
| 86 | Clostridium chauvoei | WP_021875658.1 | + | − |
| 87 | Thermoanaerobacterium | WP_014757178.1 | + | − |
| | aotearoense | | | |
| 88 | Ruminococcus sp. AT10 | WP_059066688.1 | + | − |
| 89 | Porphyromonas sp. HMSC077F02 | WP_070707924.1 | − | − |
| 90 | Acetobacterium dehalogenans | WP_026396046.1 | + | − |
| 91 | Spirochaeta alkalica | WP_018526526.1 | + | − |
| 92 | Alistipes sp. ZOR0009 | WP_047449305.1 | − | − |
| 93 | Clostridiisalibacter paucivorans | WP_026895448.1 | − | − |
| 94 | Clostridium caminithermale DSM | WP_073149471.1 | + | + |
| | 15212 | | | |
| 95 | Caldanaerobius fijiensis | WP_073341480.1 | + | − |
| 96 | Clostridium kluyveri | WP_073539833.1 | − | − |
| 97 | Pelosinus fermentans | WP_007958399.1 | + | − |
| 98 | Halanaerobium saccharolyticum | WP_005487288.1 | − | − |
| | subsp. saccharolyticum DSM 6643 | | | |
| 99 | Anaeroarcus burkinensis DSM | WP_018702299.1 | − | − |
| | 6283 | | | |
| 100 | Blautia wexlerae | WP_026648408.1 | + | − |
| 101 | Paenibacillus sp. OSY-SE | WP_019424162.1 | + | − |
| 102 | Brachyspira intermedia PWSA | WP_014488056.1 | − | − |
| 103 | Spirochaetes bacterium | OHD32879.1 | + | − |
| | GWC2_52_13 | | | |
| 104 | Thermoanaerobacterales bacterium | KUK31085.1 | − | − |
| | 50_218 | | | |
| 105 | Cohaesibacter marisflavi | WP_090072157.1 | − | − |
| 106 | Gracilibacillus ureilyticus | WP_089739945.1 | − | − |
| 107 | Romboutsia lituseburensis DSM | WP_092724914.1 | + | − |
| 108 | uncultured Clostridium sp. | SCJ29526.1 | − | − |
| 109 | Clostridium sp. CAG: 448 | CDC62685.1 | + | − |
| 110 | Clostridium ultunense Esp | CCQ95129.1 | − | − |
| 111 | Yersinia bercovieri ATCC 43970 | WP_005274635.1 | + | − |
| 112 | Proteocatella sphenisci | WP_028829945.1 | + | − |
| 113 | Clostridium sp. MSTE9 | WP_009063988.1 | − | − |
| 114 | Spirochaeta africana | WP_014454236.1 | − | − |
| 115 | Deltaproteobacteria bacterium | OGQ13386.1 | − | − |
| | RIFCSPHIGHO2_02_FULL_40_11 | | | |
| 116 | Clostridiales bacterium | KKM11466.1 | − | − |
| | PH28_bin88 | | | |
| 117 | Pelosinus propionicus DSM | WP_090932308.1 | + | − |
| 118 | Propionispora vibrioides | WP_091747803.1 | − | − |
| 119 | Natronincola ferrireducens | WP_090549432.1 | − | − |
| 120 | uncultured Ruminococcus sp. | WP_112331601.1 | − | − |
| 121 | Firmicutes bacterium CAG: 41 | WP_022229858.1 | − | − |
| 122 | Tannerella sp. oral | ETK11816.1 | − | − |
| 123 | Clostridium sp. DL-VIII | WP_009171375.1 | − | − |
| 124 | Desulfobulbus japonicus | WP_028581706.1 | − | − |
| 125 | Veillonella sp. oral | WP_009353657.1 | − | − |
| 126 | Bacillus selenitireducens | WP_013174003.1 | − | − |
| 127 | Deltaproteobacteria bacterium | OGP02283.1 | − | − |
| | GWA2_38_16 | | | |
| 128 | Clostridiaceae bacterium BRH | KJS20094.1 | − | − |
| 129 | Clostridium cadaveris | WP_035770223.1 | − | − |
| 130 | Vibrio hangzhouensis | WP_103880502.1 | − | − |
| 131 | Halanaerobium congolense | SDI24694.1 | − | − |
| 132 | uncultured Eubacterium sp. | SCH28733.1 | − | − |
| 133 | Oscillibacter sp. CAG: 241 | CDB26907.1 | − | − |
| 134 | Clostridium sp. KLE | ERI68946.1 | + | − |
| 135 | Caldalkalibacillus thermarum | WP_007505383.1 | + | − |
| | TA2.A1 | | | |
| 136 | Budvicia aquatica | WP_029095874.1 | − | − |
| 137 | Caldalkalibacillus thermarum | WP_007505383.1 | + | − |
| | TA2.A1 | | | |
| 138 | Rhodospirillum rubrum ATCC | WP_011388669.1 | − | − |
| | 11170 | | | |
| 139 | Bacteroidetes bacterium | OFX78235.1 | − | − |
| | GWE2_39_28 | | | |
| 140 | Desulfosporosinus sp. BICA1 | KJS46946.1 | − | − |
| 141 | Clostridium uliginosum | WP_090094411.1 | − | − |
| 142 | Pseudobutyrivibrio sp. ACV-2 | WP_090301343.1 | − | − |
| 143 | Sporolituus thermophilus DSM | WP_093690468.1 | − | − |
| 144 | Eubacteriaceae bacterium | WP_087275421.1 | − | − |
| | CHKC1004 | | | |
| 145 | Blautia sp. CAG: 257 | CDA04862.1 | + | − |
| 146 | Listeria marthii FSL | EFR88049.1 | + | − |
| 147 | Desulfosporosinus sp. OT | WP_009624792.1 | − | − |
| 148 | Clostridium methoxybenzovorans | WP_024346771.1 | + | − |
| 149 | Bacillus sp. m3-13 | WP_010197697.1 | + | − |
| 150 | bacterium CG2_30_54_10 | OIP28307.1 | + | − |
| 151 | Halanaerobium sp. 4-GBenrich | ODS50009.1 | − | − |
| 152 | Candidates Izimaplasma sp. | KFZ26741.1 | + | + |
| 153 | Desulfotomaculum guttoideum | WP_092244224.1 | − | − |
| 154 | Bacillus daliensis | WP_090843272.1 | − | − |
| 155 | Sporomusa acidovorans | WP_093796665.1 | − | − |
| 156 | Clostridium sp. C105KSO15 | WP_089994985.1 | − | − |
| 157 | Firmicutes bacterium CAG: 41 | CCZ36420.1 | + | − |
| 158 | Fusobacterium nucleatum subsp. | WP_085057258.1 | + | − |
| 159 | Thermoanaerobacterium | WP_013788835.1 | + | − |
| | xylanolyticum LX-11 | | | |
| 160 | Enterococcus pallens | WP_010758150.1 | − | − |
| 161 | Porphyromonas uenonis | WP_007364879.1 | − | − |
| 162 | Tenericutes bacterium | OHE32257.1 | − | − |
| | GWD2_38_27 | | | |
| 163 | Clostridia bacterium BRH_c25 | KUO67763.1 | − | − |
| 164 | Listeria monocytogenes | WP_012951491.1 | + | − |
| 165 | Clostridium lavalense | WP_092361844.1 | + | − |
| 166 | Acetanaerobacterium elongatum | WP_092640331.1 | + | − |
| 167 | Alkaliphilus peptidifermentans | WP_091539210.1 | + | − |
| | DSM | | | |
| 168 | Clostridium sp. C105KSO15 | WP_089983798.1 | − | − |
| 169 | Ruminococcus sp. CAG: 17 | CCY97458.1 | − | − |
| 170 | Clostridium hylemonae DSM 15053 | EEG72288.1 | − | − |
| 171 | Acetonema longum DSM 6540 | EGO64744.1 | − | − |
| 172 | Brachyspira innocens | WP_020003501.1 | − | − |
| 173 | Clostridium saccharobutylicum | WP_022747467.1 | − | − |
| 174 | Tenericutes bacterium | OHE28831.1 | − | − |
| | GWD2_38_27 | | | |
| 175 | Bacillus sp. FJAT-25547 | WP_053476394.1 | − | − |
| 176 | Clostridium populeti | WP_092561044.1 | + | − |
| 177 | Natronincola peptidivorans | WP_090442614.1 | − | − |
| 178 | Megasphaera paucivorans | WP_091652222.1 | − | − |
| 179 | Anaerobium acetethylicum | WP_091232027.1 | − | − |
| 180 | Eubacterium limosum | ALU13318.1 | − | − |
| 181 | Porphyromonas sp. CAG: 1061 | CCY08492.1 | − | − |
| 182 | Clostridium beijerinckii strain | AAD31841.1 | − | − |
| | NRRL B593 | | | |
| 183 | Clostridium sticklandii DSM 519 | WP_013360893.1 | − | − |
| 184 | Bacillus oryziterrae | WP_017754440.1 | − | − |
| 185 | Yersinia enterocolitica | WP_005157703.1 | − | − |
| 186 | Syntrophobacterales bacterium | OHE18777.1 | − | − |
| | GWC2_56_13 | | | |
| 187 | Candidates Bacteroides | KQM08700.1 | + | − |
| | periocalifornicus | | | |
| 188 | Anaerocolumna aminovalerica | WP_091689178.1 | + | − |
| 189 | Natronincola peptidivorans | WP_090439673.1 | − | − |
| 190 | Dendrosporobacter quercicolus | WP_092070189.1 | | − |
| 191 | uncultured Flavonifractor sp. | SCJ32847.1 | − | − |
| 192 | Geobacillus sp. Y4.1MC1 | OUM85091.1 | − | − |
| 193 | Clostridium bolteae CAG: 59 | CCX97030.1 | − | − |
| 194 | Roseburia inulinivorans A2-194 | WP_118109132.1 | − | − |

In some embodiments, the TA enzymes have catalytic efficiency and activity for the adipate semialdehyde substrate similar to the succinic semialdehyde substrate. In some embodiments, the TA enzymes are capable of converting adipate semialdehyde to 6-aminocaproate. In some embodiments, the TA enzymes are as shown in Table 2A and 2B3.

TABLE 2A

| | | | | | In vivo |
|---|---|---|---|---|---|
| | | Accession NO./ | Active | Active | 6ACA |
| Homolog # | Organism | SEQ ID NO: | 6ACA | GABA | Production |
| 1 | Achromobacter | WP_013395096.1/ | ++ | ++ | + |
| | xylosoxidans | SEQ ID NO: 30 | | | |
| 2 | Metagenomic | HAC67230.1 | − | ++ | |
| 3 | Metagenomic | WP_100856578.1 | ++ | ++ | + |
| 4 | Metagenomic | WP_025513437.1 | ++ | ++ | |
| 5 | Metagenomic | WP_056075635.1 | ++ | ++ | |
| 6 | Metagenomic | WP_017521342.1 | + | ++ | |
| 7 | Metagenomic | WP_017201530.1 | ND | ND | |
| 8 | Metagenomic | WP_059316136.1 | + | ++ | |
| 9 | Metagenomic | WP_024817707.1 | ++ | ++ | |
| 10 | Metagenomic | WP_093015681.1 | + | ++ | |
| 11 | Metagenomic | WP_069902234.1 | − | ++ | |
| 12 | Metagenomic | WP_009357183.1 | ++ | ++ | |
| 13 | Paenarthrobacter | SEQ ID NO: 31 | + | ++ | + |
| | aurescens TC1 | | | | |
| 14 | Metagenomic | WP_052967527.1 | − | ++ | |
| 15 | Metagenomic | SEQ ID NO: 32 | + | ++ | |
| 16 | Metagenomic | WP_043819139.1 | − | + | |
| 17 | Metagenomic | WP_062073360.1 | + | ++ | |
| 18 | Metagenomic | WP_015937959.1 | + | ++ | |
| 19 | Metagenomic | WP_078774761.1 | + | ++ | |
| 20 | Metagenomic | WP_046815379.1 | + | ++ | |
| 21 | Metagenomic | WP_090288152.1 | − | + | |
| 22 | Metagenomic | WP_056075635.1 | ND | ND | |
| 23 | Metagenomic | WP_003220503.1 | − | − | |
| 24 | Metagenomic | WP_003255866.1 | − | + | |
| 25 | Metagenomic | WP_104171925.1 | − | + | |
| 26 | Metagenomic | WP_123429804.1 | ++ | ++ | + |
| 27 | Metagenomic | WP_030536079.1 | ++ | ++ | + |
| 28 | Metagenomic | WP_040078233.1 | + | ++ | |
| 29 | Metagenomic | EQM68262.1 | − | − | |
| 30 | Metagenomic | WP_026539700.1 | ++ | ++ | |
| 31 | Metagenomic | WP_012250302.1 | ++ | ++ | + |
| 32 | Metagenomic | WP_007983072.1 | − | − | |
| 33 | Metagenomic | WP_045059477.1 | − | + | |
| 34 | Metagenomic | WP_093432282.1 | − | ++ | |
| 35 | Metagenomic | WP_125050266.1 | − | ++ | |
| 36 | Metagenomic | SEQ ID NO: 33 | − | ++ | |
| 37 | Metagenomic | WP_134929664.1 | − | − | |
| 38 | Metagenomic | WP_056075635.1 | ++ | ++ | |
| 39 | Metagenomic | WP_065926318.1 | − | − | |
| 40 | Metagenomic | WP_123366550.1 | − | − | |
| 41 | Metagenomic | WP_019473946.1 | − | ++ | |
| 42 | Metagenomic | WP_065926966.1 | − | ++ | |
| 43 | Metagenomic | WP_121733815.1 | − | ++ | |
| 44 | Metagenomic | WP_015937959.1 | − | − | |
| 45 | Metagenomic | WP_071291640.1 | + | + | |
| 46 | Metagenomic | WP_111045681.1 | − | − | |
| 47 | Metagenomic | WP_056737712.1 | + | ++ | |
| 48 | Metagenomic | WP_042650496.1 | − | ++ | |
| 49 | Metagenomic | WP_106886181.1 | + | ++ | |
| 50 | Metagenomic | WP_056563276.1 | ++ | ++ | + |
| 51 | Metagenomic | WP_017197131.1 | + | ++ | |
| 52 | Metagenomic | WP_099790225.1 | + | ++ | + |
| 53 | Metagenomic | WP_048393168.1 | − | + | |
| 54 | Metagenomic | WP_123366550.1 | − | ++ | |
| 55 | Metagenomic | WP_017391017.1 | + | ++ | |
| 56 | Metagenomic | SEQ ID NO: 34 | + | ++ | |
| 57 | Metagenomic | WP_056075635.1 | − | − | |
| 58 | Metagenomic | WP_054597933.1 | − | − | |
| 59 | Metagenomic | WP_095746204.1 | − | − | |
| 60 | Metagenomic | WP_003220503.1 | − | − | |
| 61 | Metagenomic | WP_017197131.1 | − | − | |
| 62 | Metagenomic | WP_052967527.1 | − | ++ | |
| 63 | Metagenomic | WP_021472307.1 | − | − | |
| 64 | Arthrobacter sp. | WP_055239729.1 | ++ | ++ | + |
| | Edens01 | | | | |
| 65 | Arthrobacter | WP_074699490.1 | − | − | |
| | crytallopoietes | | | | |
| 66 | Arthrobacter sp. KI72 | WP_079941468.1 | − | ++ | |
| 67 | Arthrobacter agilis | WP_087028133.1 | + | ++ | |
| 68 | Metagenomic | WP_119966131.1 | + | ND | |
| 69 | Metagenomic | RIK89039.1 | + | ND | |
| 70 | Metagenomic | WP_016152939.1 | − | ND | |
| 71 | Metagenomic | WP_089085686.1 | + | ND | |
| 72 | Metagenomic | WP_054094475.1 | − | ND | |
| 73 | Metagenomic | WP_105516444.1 | + | ++ | |
| 74 | Metagenomic | WP_123787193.1 | ++ | ++ | |
| 75 | Metagenomic | WP_088966344.1 | − | ND | |
| 76 | Metagenomic | WP_119775651.1 | + | ND | |
| 77 | Metagenomic | SEQ ID NO: 35 | + | ++ | |
| 78 | Metagenomic | WP_011748622.1 | ++ | ND | |
| 79 | Metagenomic | WP_111824632.1 | ++ | ++ | |
| 80 | Metagenomic | HAU57754.1 | + | ND | |
| 81 | Metagenomic | SHM39441.1 | ++ | ++ | |
| 82 | Metagenomic | WP_043683590.1 | + | ND | |
| 83 | Metagenomic | SEQ ID NO: 36 | − | ND | |
| 84 | Metagenomic | WP_121579846.1 | + | ND | |
| 85 | Metagenomic | WP_101193102.1 | + | ND | |
| 86 | Metagenomic | WP_012249988.1 | + | ++ | |
| 87 | Metagenomic | WP_119775651.1 | − | ND | |
| 88 | Metagenomic | OGB44516.1 | + | ND | |
| 89 | Metagenomic | WP_102820733.1 | + | ND | |
| 90 | Metagenomic | SHM39441.1 | + | ++ | |
| 91 | Metagenomic | SHM39441.1 | ++ | ++ | |
| 92 | Metagenomic | WP_088966344.1 | + | ++ | |
| 93 | Metagenomic | RIK89039.1 | + | ND | |
| 94 | Metagenomic | WP_043683590.1 | + | ND | |
| 95 | Metagenomic | WP_137155920.1 | + | ND | |
| 96 | Metagenomic | WP_099059309.1 | + | ++ | |
| 97 | Metagenomic | WP_101193102.1 | ND | ND | |
| 98 | Metagenomic | WP_043683590.1 | + | ND | |
| 99 | Metagenomic | WP_062685190.1 | + | ++ | |
| 100 | Metagenomic | WP_081768742.1 | + | ND | |
| 101 | Metagenomic | ODT51733.1 | + | ND | |
| 102 | Metagenomic | WP_104959793.1 | + | ND | |
| 103 | Metagenomic | WP_043683590.1 | + | ND | |
| 104 | Metagenomic | HAU57754.1 | + | ND | |
| 105 | Metagenomic | WP_009615038.1 | − | ND | |
| 106 | Metagenomic | SHM39441.1 | ++ | ++ | |
| 107 | Metagenomic | WP_056323744.1 | + | ++ | |
| 108 | Metagenomic | WP_056323744.1 | ++ | ++ | |
| 109 | Metagenomic | WP_088966344.1 | + | ++ | |
| 110 | Metagenomic | SHM39441.1 | + | ND | |
| 111 | Metagenomic | WP_125868825.1 | + | ND | |
| 112 | Metagenomic | SEQ ID NO: 37 | + | ND | |
| 113 | Metagenomic | WP_124644434.1 | + | ND | |
| 114 | Metagenomic | WP_101193102.1 | + | ND | |
| 115 | Metagenomic | WP_057067610.1 | + | ND | |
| 116 | Metagenomic | ODT32214.1 | ++ | ++ | |
| 117 | Metagenomic | WP_136961719.1 | + | ND | |
| 118 | Metagenomic | WP_093109347.1 | − | ND | |
| 119 | Metagenomic | WP_043683590.1 | + | ND | |
| 120 | Metagenomic | WP_043683590.1 | − | ND | |
| 121 | Metagenomic | WP_008955628.1 | + | ND | |
| 122 | Metagenomic | SHM39441.1 | + | ND | |
| 123 | Metagenomic | WP_003832711.1 | + | ND | |
| 124 | Metagenomic | WP_116003699.1 | + | ND | |
| 125 | Metagenomic | WP_120809350.1 | − | ND | |
| 126 | Metagenomic | WP_026133539.1 | + | ++ | |
| 127 | Metagenomic | WP_119775651.1 | + | ND | |
| 128 | Metagenomic | SHM39441.1 | + | ++ | |
| 129 | Metagenomic | WP_043683590.1 | + | ND | |
| 130 | Metagenomic | WP_015475018.1 | − | ND | |
| 131 | Metagenomic | WP_047290758.1 | ND | ND | |
| 132 | Metagenomic | WP_116810265.1 | − | ND | |
| 133 | Saccharomyces | SEQ ID NO: 38 | + | ++ | |
| | cerevisiae | | | | |
| 134 | Clostridium viride | SEQ ID NO: 39 | + | ++ | |
| 135 | Gulosibacter | SEQ ID NO: 40 | + | ++ | |
| | molinativorax | | | | |
| 136 | Kocuria rhizophila | SEQ ID NO: 41 | ++ | ++ | |
| 137 | Arthrobacter sp. FB24 | SEQ ID NO: 42 | ++ | ++ | |
| 138 | Pseudomonas sp. AAC | SEQ ID NO: 43 | + | + | |
| 139 | Pseudomonas putida | SEQ ID NO: 44 | + | + | |
| | KG-4 | | | | |
| 140 | Alcaligenes A. | SEQ ID NO: 45 | + | + | |
| | faecalis | | | | |

TABLE 2B
Transaminase variants

| Variant | | Active | Active | Active |
|---|---|---|---|---|
| # | Mutations to SEQ ID NO: 30 | 6ACA | GABA | Ala |
| 1 | A113V | + | ++ | ND |
| 2 | E112K | − | − | ND |
| 3 | I49V | + | ++ | ND |
| 4 | T264S | + | ++ | ND |
| 5 | G19R C22S D70N L186V K318M G336S S416Y | + | ++ | ND |
| 6 | V111A | + | ++ | ND |
| 7 | I203L | + | ++ | ND |
| 8 | T148I | + | ++ | ND |
| 9 | G19R D70N D99E L186V K318M G336S S416N | + | ++ | ND |
| 10 | T109S | + | ++ | ND |
| 11 | T148V | + | ++ | ND |
| 12 | S136A | ++ | ++ | ND |
| 13 | Q208R | + | ++ | ND |
| 14 | L386V | + | ++ | ND |
| 15 | G144C | + | ++ | ND |
| 16 | I49V S136A T148I | + | ++ | ND |
| 17 | S136C | + | ++ | ND |
| 18 | S136G | + | ++ | ND |
| 19 | I204K | + | ++ | ND |
| 20 | M265C | + | ++ | ND |
| 21 | V207E | + | ++ | ND |
| 22 | S136A T148I V207E | ++ | ++ | ND |
| 23 | V207T | + | ++ | ND |
| 24 | I204Q | + | ++ | ND |
| 25 | I204T | + | ++ | ND |
| 26 | L386C | + | ++ | ND |
| 27 | M265N | + | ++ | ND |
| 28 | G19R D70N L186V K318M G336S S416Y | + | ++ | ND |
| 29 | A237T | + | ++ | ND |
| 30 | A237D | + | ++ | ND |
| 31 | A237V | + | ++ | ND |
| 32 | A237G | + | ++ | ND |
| 33 | A237S | ++ | ++ | ND |
| 34 | G243C | + | ++ | ND |
| 35 | I49V S136A T148I V207E | ++ | ++ | ND |
| 36 | M265A | + | ++ | ND |
| 37 | T216V | + | ++ | ND |
| 38 | G19R D70N F133L L186V K318M G336S S416Y | + | ++ | ND |
| 39 | T216A | + | ++ | ND |
| 40 | G19R C22S D70N L186V K318M G336S S416Y | + | ++ | ND |
| 41 | T216C | + | ++ | ND |
| 42 | T242A | + | ++ | ND |
| 43 | T264P | + | ++ | ND |
| 44 | F137W Y154N | + | ++ | ND |
| 45 | Y154N | + | ++ | ND |
| 46 | Y154I | + | ++ | ND |
| 47 | G19R D70N L186V K318M G336S S416D | + | ++ | ND |
| 48 | Y154L | + | ++ | ND |
| 49 | Y154V | + | ++ | ND |
| 50 | Y154F | + | ++ | ND |
| 51 | Y154T | + | ++ | ND |
| 52 | Y154C | + | + | ND |
| 53 | Y154M | + | ++ | ND |
| 54 | S136A T148I | ++ | ++ | ND |
| 55 | F137T Y154K | − | ND | + |
| 56 | F137I | − | ND | + |
| 57 | G19R D70N K150R L186V L234I K318M G336S V390D S416N | + | ++ | ND |
| 58 | T148D | + | ND | + |
| 59 | L386A | + | ND | + |
| 60 | R94H S178T A315V R338L | + | ++ | ND |
| 61 | K226T R338L | + | ++ | ND |
| 62 | R338L | + | ++ | ND |
| 63 | L386C | + | ND | + |
| 64 | G19R D70N L186V K318M G336S A406E S416Y | + | ++ | ND |
| 65 | G19R D70N L186V K318M G336S L386P S416Y A421E | + | ++ | ND |

The non-naturally occurring microbial organisms are constructed using methods well known in the art as exemplified herein to exogenously express at least one nucleic acid encoding a 6-aminocaproic acid, caprolactam, hexamethylenediamine or levulinic acid pathway enzyme in sufficient amounts to produce 6-aminocaproic acid, caprolactam, hexamethylenediamine or levulinic acid. It is understood that the microbial organisms are cultured under conditions sufficient to produce 6-aminocaproic acid, caprolactam, hexamethylenediamine or levulinic acid. Following the teachings and guidance provided herein, the non-naturally occurring microbial organisms can achieve biosynthesis of 6-aminocaproic acid, caprolactam, hexamethylenediamine or levulinic acid resulting in intracellular concentrations between about 0.1-200 mM or more. Generally, the intracellular concentration of 6-aminocaproic acid, caprolactam, hexamethylenediamine or levulinic acid is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. Pat. No. 7,947,483, issued May 24, 2011. Any of these conditions can be employed with the non-naturally occurring microbial organisms as well as other anaerobic conditions well known in the art. Under such anaerobic conditions, the 6-aminocaproic acid, caprolactam, hexamethylenediamine or levulinic acid producers can synthesize 6-aminocaproic acid, caprolactam, hexamethylenediamine or levulinic acid at intracellular concentrations of 5-10 mM or more as well as all other concentrations exemplified herein. It is understood that, even though the above description refers to intracellular concentrations, 6-aminocaproic acid, caprolactam, hexamethylenediamine or levulinic acid producing microbial organisms can produce 6-aminocaproic acid, caprolactam, hexamethylenediamine or levulinic acid intracellularly and/or secrete the product into the culture medium.

The culture conditions can include, for example, liquid culture procedures as well as fermentation and other large scale culture procedures. As described herein, particularly useful yields of the biosynthetic products can be obtained under anaerobic or substantially anaerobic culture conditions.

As described herein, one exemplary growth condition for achieving biosynthesis of 6-aminocaproic acid, caprolactam, hexamethylenediamine or levulinic acid includes anaerobic culture or fermentation conditions. In certain embodiments, the non-naturally occurring microbial organisms can be sustained, cultured or fermented under anaerobic or substantially anaerobic conditions. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N2/CO2 mixture or other suitable non-oxygen gas or gases.

The culture conditions described herein can be scaled up and grown continuously for manufacturing of 6-aminocaproic acid, caprolactam, hexamethylenediamine or levulinic acid. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of 6-aminocaproic acid, caprolactam, hexamethylenediamine or levulinic acid. Generally, and as with non-continuous culture procedures, the continuous and/or near-continuous production of 6-aminocaproic acid, caprolactam, hexamethylenediamine or levulinic acid will include culturing a non-naturally occurring 6-aminocaproic acid, caprolactam, hexamethylenediamine or levulinic acid producing organism in sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, organisms can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of 6-aminocaproic acid, caprolactam, hexamethylenediamine or levulinic acid can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the 6-aminocaproic acid, caprolactam, hexamethylenediamine or levulinic acid producers for continuous production of substantial quantities of 6-aminocaproic acid, caprolactam, hexamethylenediamine or levulinic acid, the 6-aminocaproic acid, caprolactam, hexamethylenediamine or levulinic acid producers also can be, for example, simultaneously subjected to chemical synthesis procedures to convert the product to other compounds or the product can be separated from the fermentation culture and sequentially subjected to chemical conversion to convert the product to other compounds, if desired. As described herein, an intermediate in the adipate pathway utilizing 3-oxoadipate, hexa-2-enedioate, can be converted to adipate, for example, by chemical hydrogenation over a platinum catalyst.

As described herein, exemplary growth conditions for achieving biosynthesis of 6-aminocaproic acid, caprolactam, hexamethylenediamine or levulinic acid includes the addition of an osmoprotectant to the culturing conditions. In certain embodiments, the non-naturally occurring microbial organisms can be sustained, cultured or fermented as described above in the presence of an osmoprotectant. Briefly, an osmoprotectant means a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. For example, as described in Example XXII, Escherichia coli in the presence of varying amounts of 6-aminocaproic acid is suitably grown in the presence of 2 mM glycine betaine. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

Successfully engineering a pathway involves identifying an appropriate set of enzymes with sufficient activity and specificity. This entails identifying an appropriate set of enzymes, cloning their corresponding genes into a production host, optimizing fermentation conditions, and assaying for product formation following fermentation. To engineer a production host for the production of 6-aminocaproic acid or caprolactam, one or more exogenous DNA sequence(s) can be expressed in a host microorganism. In addition, the microorganism can have endogenous gene(s) functionally deleted. These modifications will allow the production of 6-aminocaproate or caprolactam using renewable feedstock.

In some embodiments minimizing or even eliminating the formation of the cyclic imine or caprolactam during the conversion of 6-aminocaproic acid to HMDA entails adding a functional group (for example, acetyl, succinyl) to the amine group of 6-aminocaproic acid to protect it from cyclization. This is analogous to ornithine formation from L-glutamate in Escherichia coli. Specifically, glutamate is first converted to N-acetyl-L-glutamate by N-acetylglutamate synthase. N-Acetyl-L-glutamate is then activated to N-acetylglutamyl-phosphate, which is reduced and transaminated to form N-acetyl-L-ornithine. The acetyl group is then removed from N-acetyl-L-ornithine by N-acetyl-L-ornithine deacetylase forming L-ornithine. Such a route is necessary because formation of glutamate-5-phosphate from glutamate followed by reduction to glutamate-5-semialdhyde leads to the formation of (S)-1-pyrroline-5-carboxylate, a cyclic imine formed spontaneously from glutamate-5-semialdhyde. In the case of forming HMDA from 6-aminocaproic acid, the steps can involve acetylating 6-aminocaproic acid to acetyl-6-aminocaproic acid, activating the carboxylic acid group with a CoA or phosphate group, reducing, aminating, and deacetylating.

### EXPERIMENTS

### Example 1. Identification of 5-carboxy-2-pentenoyl-CoA Reductases

Genes encoding 5-carboxy-2-pentenoyl-CoA reductases (Ter) were identified from the literature and bioinformatically from public databases using a basic local alignment search tool (BLAST) (Table 3). Genes encoding each of the reductases were synthesized, expressed in E. coli, and evaluated for 5-carboxy-2-pentenoyl-CoA (CPCoA) reductase activity using an enzyme coupled assay.

The genes encoding the TER enzyme candidates of Table 3 were cloned into a low-copy vector under a constitutive promoter and the constructs were transformed into E. coli using standard techniques. Transformants were cultured in LB medium in the presence of antibiotic overnight at 35° C., after which the cells were spun down at 15,000 rpm at room temperature. To make lysates, the supernatants were removed and E. coli cells expressing the Ter gene were resuspended in a chemical lysis solution containing lysozyme, nuclease, and 10 mM DTT. Lysates were used immediately.

The materials used in the coupled assay included three purified enzymes: 3-oxoadipyl-CoA thiolase (“thiolase” or Thl), 3-oxoadipyl-CoA dehydrogenase (“Hbd”), and 3-oxoadipyl-CoA dehydratase (“crotonase” or Crt), and the substrates included: succinyl-CoA and acetyl-CoA to generate CPCoA. Lysates of E. coli expressing the Ter genes were tested for their activity in the presence of these Thl, Hbd, and Crt substrate-producing enzymes.

The enzyme coupled assay was made up of three components:
1) the substrate solution containing 0.1 M Tris-HCl, pH 7.5, 1 mM acetyl-CoA, 1 mM succinyl-CoA, 0.25 mM NADH, and 0.25 mM NADPH;
2) the coupling enzyme mix, which contained 5 μM Thl, 1 μM Hbd, and 1 μM Crt; and
3) the TER lysate.

Acetyl-CoA, succinyl-CoA, NADH, and NADPH were purchased from Sigma.

The assay was initiated with the simultaneous addition of the coupling enzyme mix (5 μL) and the TER lysate (5 μL) to the substrate solution (30 μL). The reaction mix was incubated at room temperature and activity was monitored by a linear decrease in absorbance of NAD(P)H at 340 nm relative to a lysate that contained no TER protein. In addition, some TER candidates were evaluated for activity on crotonyl-CoA. Crotonyl-CoA was purchased from a commercial supplier. TER enzyme candidates that were significantly active relative to the wild-type control (Candida tropicalis, SEQ ID NO: 1) were defined as positive (+) and little to no detectable activity was designated as a minus (−). ND=not determined.

TABLE 3
5-carboxy-2-pentenoyl-CoA reductases.

| | | | | Activity | Activity |
|---|---|---|---|---|---|
| Candidate # | Species | SEQ ID NO. | Accession No | CP-CoA | Cr-CoA |
| 1 | Candida tropicalis | SEQ ID NO: 1 | XP_026596596.1/ | + | + |
| 2 | Coraliomargarita | | WP_013043307.1 | − | ND |
| | akajimensis | | | | |
| | DSM 45221 | | | | |
| 3 | Vibrio | | WP_009599222.1 | − | ND |
| | caribbenthicus | | | | |
| | ATCC BAA-2122 | | | | |
| 4 | Cephaloticoccus | SEQ ID NO: 4 | WP_068629633.1 | + | ND |
| | primus | | | | |
| 5 | Opitutaceae TSB47 | SEQ ID NO: 5 | WP_068772922.1 | + | ND |
| 6 | Puniceicoccaceae | SEQ ID NO: 6 | PDH30576.1 | + | ND |
| | MEDG31 | | | | |
| 7 | Rubritalea marina | SEQ ID NO: 7 | WP_018969548.1 | + | ND |
| 8 | Rubritalea | SEQ ID NO: 8 | WP_105041881.1 | + | ND |
| | profundi | | | | |
| 9 | Rubritalea | SEQ ID NO: 9 | SHK09753.1 | + | ND |
| | squalenifa | | | | |
| 10 | Verrucomicrobiae | | PAW64593.1 | − | ND |
| | bacterium | | | | |
| 11 | Vibrio hepatarius | SEQ ID NO: 11 | WP_053410267.1 | + | ND |
| 12 | Vibrio natriegens | | WP_065302224.1 | − | ND |
| 13 | Vibrio owensii | | WP_009705827.1 | − | ND |
| | LMG 25430 | | | | |
| 14 | Vibrio sp. EJY3 | SEQ ID NO: 14 | WP_014231565.1 | + | ND |
| 15 | Arabidopsis | SEQ ID NO: 15 | CAB75790.1 | + | ND |
| | thaliana | | | | |
| 16 | Rattus norvegicus | | NP_058905.1 | − | ND |
| 17 | Coraliomargarita | SEQ ID NO: 17 | OUU72757.1 | + | ND |
| | sp. | | | | |
| 18 | Haloferula sp. | SEQ ID NO: 18 | WP_035604676.1 | + | ND |
| 19 | Opitutaceae | SEQ ID NO: 19 | KRP37089.1 | + | ND |
| | BACL24 | | | | |
| 20 | Opitutaceae EW11 | SEQ ID NO: 20 | WP_107744621.1 | + | ND |
| 21 | Puniceicoccaceae | SEQ ID NO: 21 | PDH31188.1 | + | ND |
| | MED-G30 | | | | |
| 22 | Terrimicrobium | SEQ ID NO: 22 | WP_075078790.1 | + | ND |
| | sacchariphilum | | | | |
| 23 | Vibrio owensii | SEQ ID NO: 23 | WP_038894812.1 | + | ND |
| | ATCC | | | | |
| 24 | Drosophila | SEQ ID NO: 24 | NP_610914.2 | + | + |
| | melanogaster | | SEQ ID NO: 24 | | |
| 25 | Homo sapiens | SEQ ID NO: 25 | NP_057095.4 | + | ND |
| 26 | Euglena gracilis | SEQ ID NO: 26 | Truncated version of | + | + |
| | | | Q5EU90.1 | | |

### Example 2. In Vivo Activity of 5-carboxy-2-pentenoyl-CoA Reductases

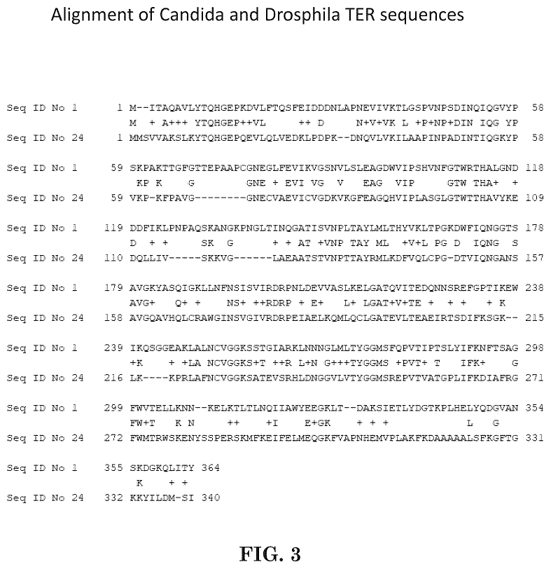

Two of the identified 5-carboxy-2-pentenoyl-CoA reductases were also tested in an in vivo assay, in which an E. coli strain that was modified to express genes encoding 3-oxoadipyl-CoA thiolase (Thl), a 3-oxoadipyl-CoA dehydrogenase (Hbd), and a 3-oxoadipyl-CoA dehydratase (“crotonase” or Crt) was transformed with a construct that included a 5-carboxy-2-pentenoyl-CoA reductase gene. A gene encoding the Candida tropicalis TER enzyme (SEQ ID NO: 1, referred to as Candidate #1) and a gene encoding the Drosophila melanogaster TER (SEQ ID NO:24, Candidate #24) were separately cloned in a low copy plasmid vector under a constitutive promoter. The amino acid sequences of the Candida and Drosophila TER are approximately 4000 identical; an amino acid sequence alignment is shown in FIG. 3. The vectors for expressing the Ter genes were transformed into the Thl/Hbd/Crt E. coli strain. Transformants were then tested for adipate production. The engineered E. coli cells were fed 2% glucose in minimal media, and after an 18 hours incubation at 35° C., the cells were harvested, and the supernatants were evaluated by analytical HPLC or standard LC/MS analytical method for adipate production. As shown in Table 4, expression of genes encoding the Candida and Drosophila TER enzymes in E. coli that included Thl, Hbd, and Crt genes resulted in adipate production by these strains.

In addition, to test the function of the Candida and Drosophila Ter genes in the 6-aminocaproate (6ACA) pathway, the constructs for expression of the Candida and Drosophila Ter genes were transformed into an E. coli strain that included, in addition to the Thl, Hbd, and Crt genes, genes encoding an aldehyde dehydrogenase (TER) and a transaminase (TA). This Thl, Hbd, Crt, TER, TA E. coli strain included all of the pathway enzymes necessary for producing 6-aminocaproate (6ACA), with the exception of the TER enzyme. The Thl, Hbd, Crt, Ter, Ald genes used are reported in in U.S. Pat. No. 8,377,680 (e. g., Example 8).

Transformation of the Thl, Hbd, Crt TER, TA E. coli strain with the Candida and Drosophila Ter constructs was performed and transformants carrying the Candida and Drosophila Ter genes were cultured for 18 hours at 35° C., as described above. After harvesting the E. coli cells by centrifugation, supernatants were analyzed for 6ACA using standard analytical methods.

TABLE 4
In vivo activity of 5-carboxy-2-pentenoyl-CoA reductases.

| | | | In vivo | In vivo |
|---|---|---|---|---|
| Candidate | | Amino acid | Adipate | ACA |
| # | Species | sequence | production | production |
| 1 | Candida | SEQ ID NO: 1 | + | + |
| | tropicalis | | | |
| 24 | Drosophila | SEQ ID NO: 24 | + | + |
| | melanogaster | | | |

Table 4 shows that the addition of the Ter gene to a strain that includes genes encoding the enzymes Thl, Hbd, Crt TER, and TA results in the production of 6ACA.

### Example 3. Targeted Mutagenesis of Ter Genes

Using structure guided design, variants of the gene encoding the Candida TER enzyme (SEQ ID NO:1) were then generated by mutating the gene encoding the Candida TER enzyme (SEQ ID NO:1) at codons for Q52, V105, S148, V149, T153, V301, T302, and N307 as well as the codons for Q11, A39, 548, S59, G97, 5103, H104, N106, F107, I147, L152, L156, R200, D201, R202, E303, K306, N308, and L316. Similarly, using structure guided design, variants ofthe gene encoding the Candida TER enzyme (SEQ ID NO:1) were then generated by mutating the gene encoding the Candida TER enzyme (SEQ ID NO: 1) at codons for S96, L98, V129, M274, T275. Substitutions at the amino acid positions were made using degenerate primer sequences and PCR, where the altered gene sequence mixtures were transformed into E. coli.

Transformants were tested in lysate assays as described in Example 1, and clones that provided lysates showing activity higher than wild type controls (SEQ ID NO:1 or SEQ ID NO: 24) in the assays were retested in triplicate lysate assays. Clones that showed higher than wild type activity were then processed for sequence analysis of the Ter genes they contained. The mutations found in the variant Ter gene sequences of the active clones are provided in Table 5. Variants demonstrating higher than wild type activity, denoted “++” or “+++”, included single mutations and combinatorial (multiple) mutations in the Ter gene.

TABLE 5
Engineered Variants of 5-carboxy-2-pentenoyl-CoA reductase

| | | Active - | Active - | Cofactor |
|---|---|---|---|---|
| Variant # | Mutations to SEQ ID NO: 1 | CPCoA | CrCoA | pref |
| 1 | None-SEQ ID NO: 1 | + | + | NADPH |
| 2 | V105G V149I V301R | +++ | ND | NADPH |
| 3 | V105N V149I V301R | +++ | ND | NADPH |
| 4 | V105R V149I V301R | +++ | ND | NADPH |
| 5 | V105K V149S V301V | ++ | ND | NADPH |
| 6 | V105R V301K | ++ | ND | NADPH |
| 7 | V149A V301K | ++ | ND | NADPH |
| 8 | V105K V149I V301R | ++ | ND | NADPH |
| 9 | V105K V149I V301L | +++ | ND | NADPH |
| 10 | V105R V149I V301K | ++ | ND | NADPH |
| 11 | Q52H | ++ | ND | NADPH |
| 12 | V105N | ++ | ND | NADPH |
| 13 | V105R | ++ | ND | NADPH |
| 14 | V105A | ++ | ND | NADPH |
| 15 | V105C | ++ | ND | NADPH |
| 16 | V149C | ++ | ND | NADPH |
| 17 | V149S | ++ | ND | NADPH |
| 18 | T153S S148R | ++ | ND | NADPH |
| 19 | T302R | +++ | ND | NADPH |
| 20 | V301M N307K | ++ | ND | NADPH |
| 21 | V301L | ++ | ND | NADPH |
| 22 | V105G V149I V301L T302R | +++ | ND | NADPH |
| 23 | V105G V149I V301K T302R | +++ | ND | NADPH |
| 24 | V105G V149I T302R | ++ | ND | NADPH |
| 25 | V105K V301I T302R | ++ | ND | NADPH |
| 26 | V105R V301L T302R | ++ | ND | NADPH |
| 27 | V105K V301L T302R | ++ | ND | NADPH |
| 28 | V105A V149I V301L T302R | ++ | ND | NADPH |
| 29 | V105G V149I V301I T302R | ++ | ND | NADPH |
| 30 | V105K V149I V301R T302R | ++ | ND | NADPH |
| 31 | V105C V149I V301R T302R | ++ | ND | NADPH |
| 32 | V105G V149I V301R T302R | +++ | ND | NADPH |
| 33 | V105G V149I T153S V301K T302R | ++ | ND | NADPH |
| 34 | V105K S148R V149S T153S V301L | ++ | ND | NADPH |
| 35 | V105G S148R V149I T153S T302R | ++ | ND | NADPH |
| 36 | V105A S148R V149S T153S | ++ | ND | NADPH |
| 37 | V105G T302R | +++ | ND | NADPH |
| 38 | V105N S148R V149S V301L N307K | +++ | ND | NADPH |
| 39 | V105K V149S V301L N307K | +++ | ND | NADPH |
| 40 | V105A S148R V149S V301L N307K | +++ | ND | NADPH |
| 41 | V105G S148R V149S T153S V301K T302R | ++ | + | NADPH |
| 42 | V105G V149I T153S T302R N307K | ++ | + | NADPH |
| 43 | V105G V149I V301L T302R N307K | ++ | + | NADPH |
| 44 | V105G S148R V149I T153S V301L T302R | ++ | + | NADPH |
| 45 | A32E V105G V149I V301R T302R | ++ | + | NADPH |
| 46 | S59C S48I V105G V149I V301R T302R | +++ | + | NADPH |
| 47 | G97R V105G N106C V149I V301R T302R | ++ | + | NADPH |
| 48 | V105G F107M V149I V301R T302R | +++ | + | NADPH |
| 49 | V105G I147V V149I V301R T302R | ++ | + | NADPH |
| 50 | V105G S148F V149I V301R T302R | ++ | + | NADPH |
| 51 | V105G V149I L152A V301R T302R | +++ | + | NADPH |
| 52 | V105G V149I L152M V301R T302R | ++ | + | NADPH |
| 53 | V105G V149I L156Y V301R T302R | +++ | + | NADPH |
| 54 | V105G V149I L156W V301R T302R | ++ | + | NADPH |
| 55 | V105G V149I V301R T302R E303N | ++ | + | NADPH |
| 56 | V105G V149I V301R T302R K306D | ++ | + | NADPH |
| 57 | S59V V105A S148R V149S V301L N307K | ++ | + | NADPH |
| 58 | S59Q V105A S148R V149S V301L N307K | ++ | + | NADPH |
| 59 | H104L V105A S148R V149S V301L N307K | ++ | + | NADPH |
| 60 | S103A V105A S148R V149S V301L N307K | ++ | + | NADPH |
| 61 | V105A S148R V149S V301L N307K L316T | ++ | + | NADPH |
| 62 | V105A S148R V149S L156F V301L N307K | ++ | + | NADPH |
| 63 | V105A S148R V149S V301L K306V N307K | ++ | + | NADPH |
| 64 | Q11H V105A S148R V149S V301L N307P | ++ | + | NADPH |
| 65 | V105A S148R V149S V301L N307V | ++ | + | NADPH |
| 66 | V105A S148R V149S V301L N307E | ++ | + | NADPH |
| 67 | V105A S148R V149S V301L N307Y | ++ | + | NADPH |
| 68 | V105A S148R V149S V301L N307L | ++ | + | NADPH |
| 69 | V105A S148R V149S V301L N307K N308D | ++ | + | NADPH |
| 70 | V105G F107M V149I R200D D201I R202D V301R | ND | + | NADH |
| | T302R | | | |
| 71 | V105G F107M V149I R200G D201I R202D V301R | ND | − | ND |
| | T302R | | | |
| 72 | V105G F107M V149I R200L D201I R202D V301R | ND | − | ND |
| | T302R | | | |
| 73 | V105G F107M V149I R200D D201L R202D V301R | ND | − | ND |
| | T302R | | | |
| 74 | V105G F107M V149I R200G D201L R202D V301R | ND | − | ND |
| | T302R | | | |
| 75 | V105G F107M V149I R200L D201L R202D V301R | ND | − | ND |
| | T302R | | | |
| 76 | V105G F107M V149I R200D D201V R202D V301R | ND | + | NADH |
| | T302R | | | |
| 77 | V105G F107M V149I R200G D201V R202D V301R | ND | + | NADH |
| | T302R | | | |
| 78 | V105G F107M V149I R200L D201V R202D V301R | ND | − | ND |
| | T302R | | | |
| 79 | V105G F107M V149I R200D D201I R202G V301R | ND | + | NADH |
| | T302R | | | |
| 80 | V105G F107M V149I R200G D201I R202G V301R | ND | − | ND |
| | T302R | | | |
| 81 | V105G F107M V149I R200L D201I R202G V301R | ND | − | ND |
| | T302R | | | |
| 82 | V105G F107M V149I R200D D201L R202G V301R | ND | + | NADH |
| | T302R | | | |
| 83 | V105G F107M V149I R200G D201L R202G V301R | ND | − | ND |
| | T302R | | | |
| 84 | V105G F107M V149I R200L D201L R202G V301R | ND | + | NADH |
| | T302R | | | |
| 85 | V105G F107M V149I R200D D201V R202G V301R | +++ | + | NADH |
| | T302R | | | |
| 86 | V105G F107M V149I R200G D201V R202G V301R | ND | − | ND |
| | T302R | | | |
| 87 | V105G F107M V149I R200L D201V R202G V301R | ND | − | ND |
| | T302R | | | |
| 88 | V105G F107M V149I R200D D201I R202L V301R | ND | − | ND |
| | T302R | | | |
| 89 | V105G F107M V149I R200G D201I R202L V301R | ND | − | ND |
| | T302R | | | |
| 90 | V105G F107M V149I R200L D201I R202L V301R | ND | − | ND |
| | T302R | | | |
| 91 | V105G F107M V149I R200D D201L R202L V301R | ND | + | NADH |
| | T302R | | | |
| 92 | V105G F107M V149I R200G D201L R202L V301R | ND | − | ND |
| | T302R | | | |
| 93 | V105G F107M V149I R200L D201L R202L V301R | ND | − | ND |
| | T302R | | | |
| 94 | V105G F107M V149I R200D D201V R202L V301R | ND | + | NADH |
| | T302R | | | |
| 95 | V105G F107M V149I R200G D201V R202L V301R | ND | − | ND |
| | T302R | | | |
| 96 | V105G F107M V149I R200L D201V R202L V301R | ND | − | ND |
| | T302R | | | |
| 97 | V105G F107M V149I R200D R202D V301R T302R | ND | + | NADH |
| 98 | V105G F107M V149I R200G R202D V301R T302R | ND | + | NADH |
| 99 | V105G F107M V149I R200L R202D V301R T302R | ND | + | NADH |
| 100 | V105G F107M V149I R200D R202G V301R T302R | +++ | + | NADH |
| 101 | V105G F107M V149I R200G R202G V301R T302R | +++ | + | NADH |
| 102 | V105G F107M V149I R200L R202G V301R T302R | +++ | + | NADH |
| 103 | V105G F107M V149I R200D R202L V301R T302R | +++ | + | NADH |
| 104 | V105G F107M V149I R200G R202L V301R T302R | ND | + | NADH |
| 105 | V105G F107M V149I R200L R202L V301R T302R | ND | + | NADH |
| 106 | V105G F107M V149I R200D V301R T302R | +++ | + | NADPH |
| 107 | V105G F107M V149I R200D R202H V301R T302R | +++ | + | NADH |
| 108 | V105G F107M V149I R200D R202S V301R T302R | +++ | + | NADH |
| 109 | V105G F107M V149I R200D R202K V301R T302R | +++ | + | NADPH |
| 110 | V105G F107M V149I R200D R202Q V301R T302R | +++ | + | |
| 111 | V105G F107M V149I R200D R202A V301R T302R | +++ | + | NADPH |
| 112 | V105G F107M V149I R200D R202C V301R T302R | ND | + | NADH |
| 113 | V105G F107M V149I R200D R202V V301R T302R | ND | + | NADH |
| | Mutations to SEQ ID NO: 24 | | | |
| 114 | S96G L98M V129I M274R T275R | + | | NADH |

Some variants were tested in duplicate lysate assays that included the preferred substrate (CPCoA or CrCoA) where one assay included only NADH as the cofactor, and the other included only NADPH as the cofactor. The relative activity of the variant in assays using each cofactor was used to determine the cofactor preference of the variants as provided in Table 5.

Table 5 shows that many of the variants had increased activity using the CPCoA substrate with respect to the wild type TER enzyme (SEQ ID NO: 1 or SEQ ID NO: 24). Many of the variants demonstrated activity using the crotonyl-CoA substrate. In addition, multiple variants having mutations at the R200 position and/or the R202 position were found to have an NADH substrate preference, unlike the wild type TER, which demonstrated a preference for NADPH as the cofactor.

### Example 4. In Vivo Activity of Variant 5-Carboxy-2-Pentenoyl-CoA Reductases

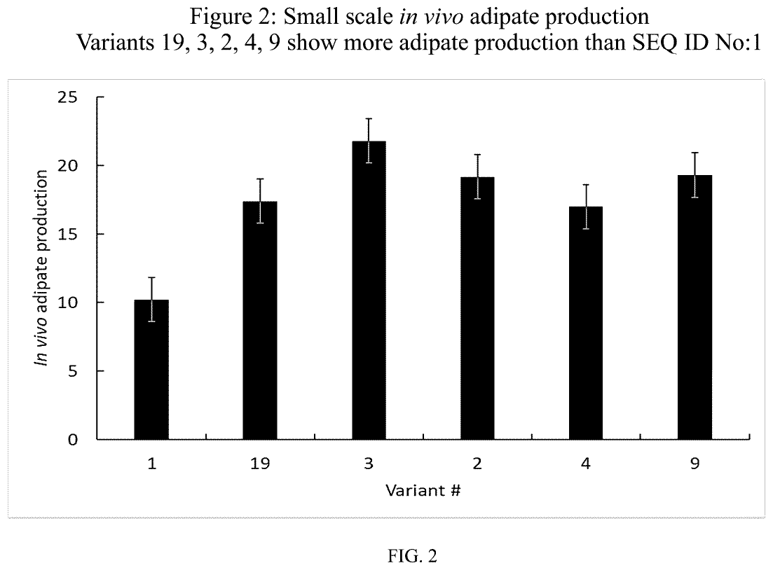

TER variants having from one to eight mutated amino acids were also assessed for their ability to function in a pathway for making adipate and a pathway for producing 6ACA using the in vivo assays described in Example 2. Many of the variants were demonstrated to work in both the adipate and the 6ACA pathways (Table 5). FIG. 2 provides an example of the assay results for the wild type Candida homolog (SEQ ID NO: 1) and Variants 2, 3, 4, 9, and 19, where the TER variant activity is provided graphically as a ratio of the wild type TER activity.

TABLE 6
Activity of TER variants in in vivo assays.

| | | in vivo | in vivo |
|---|---|---|---|
| Variant | | adipate | 6ACA |
| # | Mutations to SEQ ID NO: 1 | production | production |
| 1 | None | + | + |
| 2 | V105G V149I V301R | ++ | ND |
| 3 | V105N V149I V301R | ++ | ND |
| 4 | V105R V149I V301R | ++ | ND |
| 5 | V105K V149S V301V | ND | ND |
| 6 | V105R V301K | ND | ND |
| 7 | V149A V301K | ND | ND |
| 8 | V105K V149I V301R | ND | ND |
| 9 | V105K V149I V301L | ++ | ND |
| 10 | V105R V149I V301K | ND | ND |
| 11 | Q52H | ND | ND |
| 12 | V105N | ND | ND |
| 13 | V105R | ND | ND |
| 14 | V105A | ND | ND |
| 15 | V105C | ND | ND |
| 16 | V149C | ND | ND |
| 17 | V149S | ND | ND |
| 18 | T153S S148R | ND | ND |
| 19 | T302R | ++ | ND |
| 20 | V301M N307K | ND | ND |
| 21 | V301L | ND | ND |
| 22 | V105G V149I V301L T302R | ++ | ND |
| 23 | V105G V149I V301K T302R | ++ | ND |
| 24 | V105G V149I T302R | ND | ND |
| 25 | V105K V301I T302R | ND | ND |
| 26 | V105R V301L T302R | ND | ND |
| 27 | V105K V301L T302R | ND | ND |
| 28 | V105A V149I V301L T302R | ND | ND |
| 29 | V105G V149I V301I T302R | ND | ND |
| 30 | V105K V149I V301R T302R | ND | ND |
| 31 | V105C V149I V301R T302R | ND | ND |
| 32 | V105G V149I V301R T302R | ++ | ND |
| 33 | V105G V149I T153S V301K T302R | ND | ND |
| 34 | V105K S148R V149S T153S V301L | ND | ND |
| 35 | V105G S148R V149I T153S T302R | ND | ND |
| 36 | V105A S148R V149S T153S | ND | ND |
| 37 | V105G T302R | ++ | ND |
| 38 | V105N S148R V149S V301L N307K | ++ | ND |
| 39 | V105K V149S V301L N307K | ++ | ND |
| 40 | V105A S148R V149S V301L N307K | ++ | ++ |
| 41 | V105G S148R V149S T153S V301K T302R | ND | ND |
| 42 | V105G V149I T153S T302R N307K | ND | ND |
| 43 | V105G V149I V301L T302R N307K | ND | ND |
| 44 | V105G S148R V149I T153S V301L T302R | ND | ND |
| 45 | A32E V105G V149I V301R T302R | ND | ND |
| 46 | S59C S48I V105G V149I V301R T302R | ++ | ND |
| 47 | G97R V105G N106C V149I V301R T302R | ND | ND |
| 48 | V105G F107M V149I V301R T302R | ++ | ++ |
| 49 | V105G I147V V149I V301R T302R | ND | ND |
| 50 | V105G S148F V149I V301R T302R | ND | ND |
| 51 | V105G V149I L152A V301R T302R | ++ | ND |
| 52 | V105G V149I L152M V301R T302R | ND | ND |
| 53 | V105G V149I L156Y V301R T302R | ND | ND |
| 54 | V105G V149I L156W V301R T302R | ++ | ND |
| 55 | V105G V149I V301R T302R E303N | ND | ND |
| 56 | V105G V149I V301R T302R K306D | ND | ND |
| 57 | S59V V105A S148R V149S V301L N307K | ND | ND |
| 58 | S59Q V105A S148R V149S V301L N307K | ND | ND |
| 59 | H104L V105A S148R V149S V301L N307K | ND | ND |
| 60 | S103A V105A S148R V149S V301L N307K | ND | ND |
| 61 | V105A S148R V149S V301L N307K L316T | ND | ND |
| 62 | V105A S148R V149S L156F V301L N307K | ND | ND |
| 63 | V105A S148R V149S V301L K306V N307K | ND | ND |
| 64 | Q11H V105A S148R V149S V301L N307P | ND | ND |
| 65 | V105A S148R V149S V301L N307V | ND | ND |
| 66 | V105A S148R V149S V301L N307E | ND | ND |
| 67 | V105A S148R V149S V301L N307Y | ND | ND |
| 68 | V105A S148R V149S V301L N307L | ND | ND |
| 69 | V105A S148R V149S V301L N307K N308D | ND | ND |
| 70 | V105G F107M V149I R200D D201I R202D V301R | ND | ND |
| | T302R | | |
| 71 | V105G F107M V149I R200G D201I R202D V301R | ND | ND |
| | T302R | | |
| 72 | V105G F107M V149I R200L D201I R202D V301R | ND | ND |
| | T302R | | |
| 73 | V105G F107M V149I R200D D201L R202D V301R | ND | ND |
| | T302R | | |
| 74 | V105G F107M V149I R200G D201L R202D V301R | ND | ND |
| | T302R | | |
| 75 | V105G F107M V149I R200L D201L R202D V301R | ND | ND |
| | T302R | | |
| 76 | V105G F107M V149I R200D D201V R202D V301R | ND | ND |
| | T302R | | |
| 77 | V105G F107M V149I R200G D201V R202D V301R | ND | ND |
| | T302R | | |
| 78 | V105G F107M V149I R200L D201V R202D V301R | ND | ND |
| | T302R | | |
| 79 | V105G F107M V149I R200D D201I R202G V301R | ND | ND |
| | T302R | | |
| 80 | V105G F107M V149I R200G D201I R202G V301R | ND | ND |
| | T302R | | |
| 81 | V105G F107M V149I R200L D201I R202G V301R | ND | ND |
| | T302R | | |
| 82 | V105G F107M V149I R200D D201L R202G V301R | ND | ND |
| | T302R | | |
| 83 | V105G F107M V149I R200G D201L R202G V301R | ND | ND |
| | T302R | | |
| 84 | V105G F107M V149I R200L D201L R202G V301R | ND | ND |
| | T302R | | |
| 85 | V105G F107M V149I R200D D201V R202G V301R | ++ | ++ |
| | T302R | | |
| 86 | V105G F107M V149I R200G D201V R202G V301R | ND | ND |
| | T302R | | |
| 87 | V105G F107M V149I R200L D201V R202G V301R | ND | ND |
| | T302R | | |
| 88 | V105G F107M V149I R200D D201I R202L V301R | ND | ND |
| | T302R | | |
| 89 | V105G F107M V149I R200G D201I R202L V301R | ND | ND |
| | T302R | | |
| 90 | V105G F107M V149I R200L D201I R202L V301R | ND | ND |
| | T302R | | |
| 91 | V105G F107M V149I R200D D201L R202L V301R | ND | ND |
| | T302R | | |
| 92 | V105G F107M V149I R200G D201L R202L V301R | ND | ND |
| | T302R | | |
| 93 | V105G F107M V149I R200L D201L R202L V301R | ND | ND |
| | T302R | | |
| 94 | V105G F107M V149I R200D D201V R202L V301R | ND | ND |
| | T302R | | |
| 95 | V105G F107M V149I R200G D201V R202L V301R | ND | ND |
| | T302R | | |
| 96 | V105G F107M V149I R200L D201V R202L V301R | ND | ND |
| | T302R | | |
| 97 | V105G F107M V149I R200D R202D V301R T302R | ND | ND |
| 98 | V105G F107M V149I R200G R202D V301R T302R | ND | ND |
| 99 | V105G F107M V149I R200L R202D V301R T302R | ND | ND |
| 100 | V105G F107M V149I R200D R202G V301R T302R | ++ | ++ |
| 101 | V105G F107M V149I R200G R202G V301R T302R | ND | ND |
| 102 | V105G F107M V149I R200L R202G V301R T302R | ++ | ++ |
| 103 | V105G F107M V149I R200D R202L V301R T302R | ++ | ++ |
| 104 | V105G F107M V149I R200G R202L V301R T302R | ND | ND |
| 105 | V105G F107M V149I R200L R202L V301R T302R | ND | ND |
| 106 | V105G F107M V149I R200D V301R T302R | ND | ND |
| 107 | V105G F107M V149I R200D R202H V301R T302R | ND | ND |
| 108 | V105G F107M V149I R200D R202S V301R T302R | ND | ND |
| 109 | V105G F107M V149I R200D R202K V301R T302R | ND | ND |
| 110 | V105G F107M V149I R200D R202Q V301R T302R | ND | ND |
| 111 | V105G F107M V149I R200D R202A V301R T302R | ND | ND |
| 112 | V105G F107M V149I R200D R202C V301R T302R | ND | ND |
| 113 | V105G F107M V149I R200D R202V V301R T302R | ND | ND |

1-72. (canceled)

73. A non-naturally occurring microbial organism comprising at least one engineered trans-enoyl CoA reductase (TER) enzyme comprising one or more alteration of an amino acid of SEQ ID NO:1 or SEQ ID NO: 24,
wherein the one or more amino acid alterations is selected from one or more positions corresponding to residues Q11, A32, S48, Q52, S59, G97, S103, H104, V105, N106, F107, I147, S148, V149, L152, T153, L156, R200, D201, R202, V301, T302, E303, K306, N307, N308, L316, or combinations thereof of SEQ ID NO:1, or
wherein the alterations of the amino acid is selected from one or more positions corresponding to S96, L98, M274, T275 or combinations thereof of SEQ ID NO: 24.

74. The non-naturally occurring microbial organism of claim 73, wherein the amino acid sequence comprises at least 60% identity to at least 25 contiguous amino acids of SEQ ID NO:1 or SEQ ID NO: 24.

75. The non-naturally occurring microbial organism of claim 73, wherein the engineered trans-enoyl CoA reductase reacts with 5-carboxyl-2-pentenoyl-CoA to form adipyl-CoA.

76. The non-naturally occurring microbial organism of claim 73, wherein the engineered TER comprises activity that is at least 20% higher than the activity of the trans-enoyl CoA reductase of SEQ ID NO:1 or SEQ ID NO: 24 with no amino acid alterations.

77. The non-naturally occurring microbial organism of claim 73, wherein the non-naturally occurring microbial organism comprises genes encoding a 3-oxoadipyl-CoA thiolase, a 3-oxoadipyl-CoA dehydrogenase, and a 3-oxoadipyl-CoA dehydratase that is able to produce at least 10% more adipate than a control cell that expresses the wild type TER from which the engineered TER is derived.

78. The non-naturally occurring microbial organism of claim 73, wherein the expressed engineered TER can further use crotonyl-CoA as a substrate.

79. The non-naturally occurring microbial organism of claim 73, wherein the expressed engineered TER has greater catalytic efficiency for 5-carboxyl-2-pentenoyl-CoA as the substrate compared to crotonyl-CoA as the substrate, as compared to the catalytic efficiency of wild-type SEQ ID Nos: 1 or 24.

80. The non-naturally occurring microbial organism of claim 73, wherein the TER is derived from a bacterial, yeast, fungal, algal, animal, or plant species.

81. The non-naturally occurring microbial organism of claim 73, wherein the non-naturally occurring microbial organism comprises two, three, four, five, six, or seven exogenous nucleic acids each encoding an enzyme for a 6-aminocaproic acid pathway, hexamethylenediamine pathway, caprolactam pathway, 1,6-hexanediol pathway, caprolactone pathway, or a combination of two or more pathways.

82. The non-naturally occurring microbial organism of claim 73, wherein the non-naturally occurring microbial organism encodes an enzyme for a 6-aminocaproic acid pathway, wherein the 6-aminocaproic acid pathway comprises: (i) transaminase, (ii) 6-aminocaproate dehydrogenase, or (iii) transaminase and 6-aminocaproate dehydrogenase enzymes.

83. A method of producing adipyl-CoA comprising:
culturing a non-naturally occurring microbial organism of claim 73 for a sufficient time period and conditions for producing adipyl-CoA.

84. The method of claim 83, further comprising recovering adipyl-CoA from the non-naturally occurring microbial organism, the fermentation broth, or both.

85. The method of claim 83, wherein the non-naturally occurring microbial organism further comprises two, three, four, five, six, or seven exogenous nucleic acids each encoding an enzyme for the 6-aminocaproic acid pathway, hexamethylenediamine pathway, caprolactam pathway, 1, 6-hexanediol pathway, caprolactone pathway, or a combination of two or more pathways.

86. The method of claim 83, comprising producing hexamethylenediamine.

87. The method of claim 83, wherein the 6-aminocaproic acid pathway comprises: (i) transaminase, (ii) 6-aminocaproate dehydrogenase, or (iii) transaminase and 6-aminocaproate dehydrogenase enzymes.


SEQUENCE LISTING (49 sequences)

SEQ ID NO: 1 - protein, 364 aa, Candida tropicalis
MITAQAVLYTQHGEPKDVLFTQSFEIDDDNLAPNEVIVKTLGSPVNPSDINQIQGVYPSKPAKTTGFGTTEPAAPCGNEGLFEVIKVGSNVLSLEAGDWVIPSHVNFGTWRTHALGNDDDFIKLPNPAQSKANGKPNGLTINQGATISVNPLTAYLMLTHYVKLTPGKDWFIQNGGTSAVGKYASQIGKLLNFNSISVIRDRPNLDEVVASLKELGATQVITEDQNNSREFGPTIKEWIKQSGGEAKLALNCVGGKSSTGIARKLNNNGLMLTYGGMSFQPVTIPTSLYIFKNFTSAGFWVTELLKNNKELKTLTLNQIIAWYEEGKLTDAKSIETLYDGTKPLHELYQDGVANSKDGKQLITY

SEQ ID NO: 2-3 - skipped (2 entries)

SEQ ID NO: 4 - protein, 393 aa, Cephaloticoccus sp.
MIIKPKVRGFVCVTAHPDGCAAHVQEWIDYVKAQPAIEGGPKKVLVIGSSTGYGLASRLALAFGARASTLGVFFERPSEEGRPATPGWYNTIALTKAARAEGLYARNIMGDAFSDEIKAQTIEAIKADLGQVDLVVYSLASPRRVHPKTGTVHKSCLKPIGAPYTNKTVDTDKGLVSTITIEPASAEEVAETVAVMGGEDWEMWIDALAAAGVLAPGAQSLAYSYIGPEVTWPIYQNGTIGMAKNDLSRAAKAIDAKLGASGGRAFISVNKALVTQASSAIPVVPLYISLLYKVMKAAGLHEGCIEQMHRLFATQVYNGNSPRFDEAGRVRIDDWEMRPEIQAAVARLWPTITTENLEAETDIAGYRAEFLKLFGFGLPGVDYEAPAEPHVPL

SEQ ID NO: 5 - protein, 396 aa, Termitidicoccus sp.
  source 1..396: note = strain TSB47
MVIKPKVRGFICVTAHPAGCAAHVQQQIDYVKSKGPVRDGPKKVLVLGASTGFGLASRVTAAFGSGAATLGVFFERPSEDGRLATPGWYNSAAFTRAALDAGLYAKNINGDAFSDAVKRQAIDLIKADLGQVDLVVYSLASPRRTHPRTGATHKSCLKPIGAPYTNKTVDTDRGIVSDVTIEPATETEIADTVAVMGGEDWEMWIDALLEAGVLAPGATSVAYSYIGPEVTWPIYKNGTIGTAKIDLERAAKKIDAALKAHGYGRAFISVNKALVTQASSAIPVVPLYISILYKIMKAKGLHEGCIEQMQRLFATQLYNGSALAFDDGGRVRVDNWEMRPDVQAEAAKIWPQVTTENLAALTDIEGYRSEFLRLFGFGLPGVNYDADYEPHVPMPE

SEQ ID NO: 6 - protein, 392 aa, Opitutia sp.
  source 1..392: note = strain MEDG31
MIVKPRIRGFVCITAHPVGCTAKVQREIEVAKAARQSGGPKKVLVIGASTGYGLSSRIAAAFGSDAATLGVFYERPSLKGKPASAGWYNSAAFESAAHTSGLYAKSINGDAFSTEVKEQTIDMIKADLGQVDLVIYSLASPRRTDPASGETYRSCLKPIGGTYTNRTLDTDKAQVSDITIDAATKQEAEDTVKVMGGEDWELWMQALAQAGVVAQGAKTVAYSYIGPEITWPVYTNGTIGAAKKDVERAAAAITAQYDCDAYVSVNKAVVTQASSAIPVVPLYISILFKIMKSKGTHEDCIEQTVRLFNQRLYGGDLQLDESGRIRIDDWEMEPEIQAAVQEIWPEISSETLIELSDFTGYQKNFLNLFGFAIEGVDYDVEVEVDVPVPSIS

SEQ ID NO: 7 - protein, 399 aa, Rubritalea marina
MIVKPKIRGFICTTAHPDGCAAHVQEQIDYVKSKPKLENGPKNVLVIGSSTGYGLASRIVPAFQSGANTLGLFFEKPGSEKRPGSAGWYNSAAFEKAANAEGLFAKSLNGDAFSNELKEQAIDIIKNEMDGKIDLVVYSLASPRRTDPNTGEVYKSVLKTTGGEYTNKTLNTDKGEIDEVTIGAATSEEIDHTIKVMGGEDWEFWMDALIEADVLDSGVKTVAYSYIGPELTWPIYTDGTIGQAKKDLESSCAKIDASLKEKFNGEAFVSVNKALVTQASSAIPVVPLYISILYKEMKAKGTHEGCIEQIERLFSDRLYSGDPKLDEAGRIRIDDWEMREEIQSAVAGIWTSVSNENFGDVADFDGYQSEFLKLFGFGLDGVDYESETDTTTAVPSLAE

SEQ ID NO: 8 - protein, 399 aa, Rubritalea profundi
MIVKPKIRGFICTTSHPDGCAAHVQEQIDYVKSKPKSENGPKNVLVIGSSTGYGLASRIVPAFGAGANTLGLFFEKPGSEKRTGSAGWYNTAAFEKAATAEGIFAKSLNGDAFSNELKQQAIDIIKNDMDGKIDLIVYSLASPRRTDPNTGEVYKSVLKTTGGEYTNKTLNTDKGEVDEVTIGAATAEEIDNTVKVMGGEDWEFWVNALADANVLAEGVKTVAYSYIGPELTWPIYTDGTIGRAKKDVEKSCAKLDAKLKASLGGEAFVSVNKALVTQASSAIPVVPLYISILYKEMKAKGLHEGCIEQIERLFSDRLYAADRKLDEAGRIRIDDLEMRDDIQAAVANIWSSVSNENFADVADFKGYKDEFLKLFGFGIDGVDYDAEIDPATGVPSLAE

SEQ ID NO: 9 - protein, 398 aa, Rubritalea squalenifa
MIVSPKIRGFICTTAHPDGCAAHVQEQIDYVKSKPKLENGPKNVLVIGASTGYGLASRIVPAFASGANTLGLFFEKPGSEKRTGSAGWYNSAAFEKAATAEGLFAKSLNGDAFSHEMKAQAVDIIKNEMGGKIDLVVYSLASPRRTDPDTGEVYKSVLKTTGGDYTNKTLNTDKGEVEEVTIGAATPEEIEHTIKVMGGEDWELWMNALSDAGVLADGVKTVAYSYIGPELTWPIYTDGTIGQAKKDLEKSCAQLNEKLSSLNGEAYVSVNKALVTQASSAIPVVPLYISILYKEMKEKGLHEGCIEQIERLFADRLYSGDAKLDEAGRIRIDDWEMRDDIQSAVAEKWKTVSNENFSEIADFQGYQDEFLKLFGFGLEGVNYEADTDPATGVPSIAE

SEQ ID NO: 10 - skipped

SEQ ID NO: 11 - protein, 400 aa, Vibrio hepatarius
MIIKPRIRGFICTTTHPVGCEANVKEQIAYTKAQGPIKNAPKRVLVVGASSGYGLSSRIAAAFGGGASTIGVFFEKEGTEKKPGTAGFYNAAAFEKLAREEGLYAKSLNGDAFSNEAKQKTIELIKEDLGQVDMVVYSLASPVRKMPETGELVRSALKPIGETYTSTAVDTNKDVIIEASVEPATEEEIKDTVTVMGGEDWELWINALSEAGVLAEGCKTVAYSYIGTELTWPIYWDGALGKAKMDLDRAATALNEKLAATGGSANVAVLKSVVTQASSAIPVMPLYIAMVFKKMREEGVHEGCMEQIYRMFSQRLYKEDGSAAEVDEKNRLRLDDWELREDIQQHCRDLWPQITTENLKELTDYVEYKEEFLKLFGFGVDGVDYEADVNPAVEADFIAI

SEQ ID NO: 12-13 - skipped (2 entries)

SEQ ID NO: 14 - protein, 400 aa, Vibrio sp.
MIIKPRIRGFICTTTHPVGCEANVKEQIAYTKAQGPIKNAPKRVLVVGASSGYGLSSRIAAAFGGGASTIGVFFEKEGTEKKPGTAGFYNAAAFEKLAREEGLYAKSLNGDAFSNEAKEKTIELIKQDLGQIDMVVYSLASPVRKLPETGELIRSSLKPIGETYTSTAVDTNKDVIIEASVEPATEEEIKDTVTVMGGEDWELWINALSEAGVLAEGCKTVAYSYIGTELTWPIYWDGALGKAKMDLDRAATALNEKLAQTGGSANVAVLKSVVTQASSAIPVMPLYIAMVFKKMREEGVHEGCMEQIYRMFSQRLYKEDGSAAEVDDMNRLRLDDWELREDIQQHCRDLWPQITTENLKELTDYVEYKEEFLKLFGFGVDGVDYEADVNPAVEADFIQI

SEQ ID NO: 15 - protein, 341 aa, Arabidopsis thaliana
METPTLCIKSFSTIMSPPSKAIVYEEHGSPDSVTRLVNLPPVEVKENDVCVKMIAAPINPSDINRIEGVYPVRPPVPAVGGYEGVGEVYAVGSNVNGFSPGDWVIPSPPSSGTWQTYVVKEESVWHKIDKECPMEYAATITVNPLTALRMLEDFVNLNSGDSVVQNGATSIVGQCVIQLARLRGISTINLIRDRAGSDEAREQLKALGADEVFSESQLNGNLPEPALGFNCVGGNAASLVLKYLREGGTMVTYGGMSKKPITVSTTSFIFKDLALRGFWLQSWLSMGKVKECREMIDYLLGLARDGKLKYETELVPFEEFPVALDKALGKLGRQPKQVITF

SEQ ID NO: 16 - skipped

SEQ ID NO: 17 - protein, 393 aa, Coraliomargarita sp.
MIVKPRIRGFVCITAHPLGCEANVRSEIEKASAAQLEGGPKKVLVIGASTGYGLSSRITAAFSHGADTLGVFFERPSMKGKPASAGWYNSVAFEKAAHESGRYAKSINGDAFSNEIKEQTIQAIKDDLGQVDLVIYSLASPRRTDPSDGETYKSCLKPLGDTYTNRTLDTDKGVVSEVSIETATAEETEHTVKVMGGEDWELWMCALADAGCLADGAKTVAYSYVGPEITWPVYTNGTIGAAKKDVERAAAAIHSNHGCEAYVSVNKAVVTQASSAIPVVPLYISILFKVMKAKGTHEGCIEQMIRLFNERLYGGGLQLDEDGRIRVDDLEMEPDVQGEVAAIWPEIQTENLDERSDFAGYRQDFLRCFGFGIEGVDYEADVEVELPLPSGNQ

SEQ ID NO: 18 - protein, 399 aa, Haloferula sp.
MIISPKIRGFICTTSHPDGCAAHVQEQIDYVKSKPPLANAPKRVLVIGSSTGYGLASRIVPAFGGGAATIGVFFEKEGSEGKTGSAGWYNSVAFEQAATKAGLYAKSFNGDAFSNEVKQQVIDAIKADLGTVDMIVYSLASPRRTDPATGEVYKSVLKPTGDTYTNKNLNTDKKQLEPVTIEPASEDEIAQTVKVMGGEDWKLWIDALSQAGVLADGFKTVAYSYIGPELTWAIYTDGTIGRAKVHLEETAKEMNAAFGAGTAVVSVNKALVTQASSAIPVVPLYISILYKIMKAKGTHEGCIEQIERLFADHYGAANGPTLDDKGRIRIDDWEMRPEIQEEVEKIWGIITTENLNELSDFAGYQKEFLKLFGFGLPGVDYAAETDSVRPLPSLSSSPA

SEQ ID NO: 19 - protein, 393 aa, Lentimonas sp.
  source 1..393: note = strain BACL24
MIVKPRIRGYVCITAHPKGCEAKVREEIEVAKATKRSDGPKRVLVLGSSTGYGLSSRIATAFGYDAATFGIFFERPSTNGKPASAGWYNSVAFEKAAHEAGLYAKSINGDAFSNEVKAQAIAQIKEDLGQIDLVVYSLASPRRTDPETGEVYKSVLKPIGDQAFTNRTMDTDRDEVKDVTIEPATEEEIQHTINVMGGQDWELWMKALNDAGVLAPGAKSVAYSYIGPEATWPVYTNGTIGQAKKDVERAAAAITKAYDCAAYVAVNKAVVTQASSAIPVVPLYISIIIKLMKEKGTHEDCIEQMVRLFDDRLYGKDLQLDELGRIRVDDWEMQPDIQAKVIEIWPSITSETLDVLGDYTGYQHNFLTLFGFGLDGVDYDEDVEVDLPLPSSL

SEQ ID NO: 20 - protein, 395 aa, unidentified
  source 1..395: note = Opitutaceae bacterium strain EW11
MVIKPKVRGFVCVTAHPAGCAAHVQEQIDYVKSKGPIKNGPKKVLVIGASTGYGLASRIAASFGSGAATIGVFFERPSEEGRPATPGWYNTIGFTKAARAAGLYAKNINGDAFSNEIKQQVVDLIKADLGQVDLIVYSLASPRRTHPKTGVVHKSVLKPVGAPYTNKTVDTDKGVVSNVTIEPAADTEVSDTIAVMGGEDWEMWIHALADAKVLAPGATSVAYSYIGPEVTWPIYKNGTIGLAKNDLEAAGRRIDALLKAHGYGRAFISVNKALVTQASSAIPVVPLYISILYKIMKAKGTHEGCIEQMQRLFATQMYNNSALHFDDGGRVRVDDWEMKPDVQAEVARLWPQVTTENIEQLTDIAGYRHEFLKLFGFGLDGIDYDADIEPHLPML

SEQ ID NO: 21 - protein, 393 aa, Coraliomargarita sp.
  source 1..393: note = strain MED-G30
MIVKPRIRGFVCITAHPQGCEANVRSEIEKASAAQLEGGPKKVLVIGASTGYGLSSRITAAFSHGADTLGVFFERPSMKGKPASAGWYNSVAFEKAAHECGRYAKSINGDAFSNEIKEQAIQAIKDDLGQVDLVIYSLASPRRTDPSDGETYKSCLKPLGDTYTNRTLDTDKGVVSEVSIETATAEETEHTVKVMGGEDWELWMRALADAGCLADGAKTVAYSYVGPEITWPVYTNGTIGAAKKDVERAAAAIHSNHGCEAYVSVNKAVVTQASSAIPVVPLYISILFKVMKAKGTHEGCIEQMIRLFNERLYGGGLQLDEDGRIRVDDLEMEPDVQGEVAAIWPEIQTENLDERSDFAGYRQDFLRCFGFGIEGVDYEADVEVELPLPSGNQ

SEQ ID NO: 22 - protein, 395 aa, Terrimicrobium sp.
MIITPKIRGFICTTAHPDGCAAHVQEQIAYVKSKPSLPNAPKRVLVVGSSTGYGLASRIVAAFGGGASTVGVFFEKEPVEKKTASAGWYNSAAFEEAALKEGLYAKSFNGDAFSNEMKQKVIETIKTDLGQIDMIVYSLASPRRTDPVTGEVFKSVLKPTGETFTNKNLNTDKKEIETVSIDPASPEEIAHTVKVMGGEDWQLWIDALENAGALAEGFKTVAYSYIGPELTWAIYTDGTIGRAKLDLEKTARDINGKFGAGTALVSVNKALVTQASSAIPVVPLYISILYKVMKAKGLHEGCIEQIERLFADHYCAPEGPKLDDAGRIRIDDWEMREDVQAEVRDAWKDVSTETIDAATDFAGYQKEFYRLFGFGLSGVDYAADVDPIRKIPSLG

SEQ ID NO: 23 - protein, 400 aa, Vibrio owensii
MIIKPRIRGFICTTTHPVGCEANVKEQIAYTKAQGPIKNAPKRVLVVGASSGYGLSSRIAAAFGGGASTIGVFFEKEGTEKKPGTAGFYNSVAFEKLAREEGLYAKSLNGDAFSNEAKQKTIDLIKEDLGQVDMVVYSLASPVRKMPETGELIRSALKPIGETYTSTAVDTNKDVIIEASVEPATEQEIQDTVTVMGGEDWELWINALSEAGVLADGCKTVAYSYIGTELTWPIYWDGALGKAKMDLDRAAAALNEKLSTTGGSANVAVLKSVVTQASSAIPVMPLYIAMVFKKMREEGVHEGCMEQIFRMFSQRLYKEDGSAAEVDEVNRLRLDDWELREDIQQHCRDLWPQITSDNLKELTDYVEYKEEFLKLFGFGVDGVDYEADVNPAVETDFTQI

SEQ ID NO: 24 - protein, 339 aa, Drosophila melanogaster
MMSVVAKSLKYTQHGEPQEVLQLVEDKLPDPKDNQVLVKILAAPINPADINTIQGKYPVKPKFPAVGGNECVAEVICVGDKVKGFEAGQHVIPLASGLGTWTTHAVYKEDQLLIVSKKVGLAEAATSTVNPTTAYRMLKDFVQLCPGDTVIQNGANSAVGQAVHQLCRAWGINSVGIVRDRPEIAELKQMLQCLGATEVLTEAEIRTSDIFKSGKLKKPRLAFNCVGGKSATEVSRHLDNGGVLVTYGGMSREPVTVATGPLIFKDIAFRGFWMTRWSKENYSSPERSKMFKEIFELMEQGKFVAPNHEMVPLAKFKDAAAAALSFKGFTGKKYILDMS

SEQ ID NO: 25 - protein, 321 aa, Homo sapiens
MPAKVVELKNLELAAVRGSDVRVKMLAAPINPSDINMIQGNYGFLPELPAVGGNEGVAQVVAVGSNVTGLKPGDWVIPANAGLGTWRTEAVFSEEALIQVPSDIPLQSAATLGVNPCTAYRMLMDFEQLQPGDSVIQNASNSGVGQAVIQIAAALGLRTINVVRDRPDIQKLSDRLKSLGAEHVITEEELRRPEMKNFFKDMPQPRLALNCVGGKSSTELLRQLARGGTMVTYGGMAKQPVVASVSLLIFKDLKLRGFWLSQWKKDHSPDQFKELILTLCDLIRRGQLTAPACSQVPLQDYQSALEASMKPFISSKQILTM

SEQ ID NO: 26 - protein, 405 aa, Euglena gracilis
MAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 27 - protein, 453 aa, Clostridium kluyveri
MSNEVSIKELIEKAKVAQKKLEAYSQEQVDVLVKALGKVVYDNAEMFAKEAVEETEMGVYEDKVAKCHLKSGAIWNHIKDKKTVGIIKEEPERALVYVAKPKGVVAATTPITNPVVTPMCNAMAAIKGRNTIIVAPHPKAKKVSAHTVELMNAELKKLGAPENIIQIVEAPSREAAKELMESADVVIATGGAGRVKAAYSSGRPAYGVGPGNSQVIVDKGYDYNKAAQDIITGRKYDNGIICSSEQSVIAPAEDYDKVIAAFVENGAFYVEDEETVEKFRSTLFKDGKINSKIIGKSVQIIADLAGVKVPEGTKVIVLKGKGAGEKDVLCKEKMCPVLVALKYDTFEEAVEIAMANYMYEGAGHTAGIHSDNDENIRYAGTVLPISRLVVNQPATTAGGSFNNGFNPTTTLGCGSWGRNSISENLTYEHLINVSRIGYFNKEAKVPSYEEIWG

SEQ ID NO: 28 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 29 - protein, 463 aa, Clostridium difficile
MEKAVENFEDLSKEYINGYIERARKAQREFECYTQEQVDKIVKIVGKVVYYNAEYLAKLAVEETGMGVYEDKVAKNKSKAKVIYNNLKDKKSVGIIDIDRETGITKVAKPVGVVAAITPCTNPIVTPMSNAMFALKGRNAIIITPHHKAIGCSTKTVEMINEELEKIGAPENLIQILDQQSRENTRNLISSADVVIATGGMGMVKAAYSSGKPALGVGAGNVQCIIDRDVDIKEAVPKIIAGRIFDNGIICSGEQSVIVAEEMFDKIMDEFKNNKGFIVRDKVQKEAFRNAMFVNKSMNKDAVGQSVHTIAKIAGVEIPEDTKIIVIEADGPGEEDIIAKEKMCPVISAYKYKSFEEGVAIAKANLNVEGKGHSVSIHSNTVKNIEYAGENIEVSRFVINQCCATSAGGSFFNGLAPTNTLGCGSWGNNSISENLDYKHLINISRIAYYMPENEVPTDEELWG

SEQ ID NO: 30 - protein, 421 aa, Achromobacter xylosoxidans
MKNQDLNTRRSLATPRGVGVMCDFYAVRAENATLWDANGKEYIDFAGGIAVLNTGHLHPKIKAAVAAQLDNFTHTAYQIVPYEGYVSLAERINRLAPIDGLKKSAFFTTGVEAVENAVKIARSATGRSGVIAFSGSFHGRTMLGMALTGKVAPYKLSFGPMPGDIYHVPFPNATQSISVADSLKALDLLFKCDIDPKRVAAIIIEPVQGEGGFNITPPELMTALRKVCDEHGILLIADEVQTGFGRTGKLFAMEHHSVQADLITMAKSLGGGFPISGVVGRADVMDAPAAGGLGGTYAGNPLAVAAAHAVLDVIAEEKLCERADALGDKLRAHLEGLRAKVPGIADVRGLGSMVALELNDPATGKPDAEAVKRVQARAIEKGLILLSCGVYGNVLRFLYPLTIPDAQFDRALAILSEALAA

SEQ ID NO: 31 - protein, 456 aa, Paenarthrobacter sp.
  source 1..456: note = strain TC1
MTTTANELSYRIEQKRNINGAFPGPKSQALAERRSAVVAAGVASGVPVYVEDADGGIIRDVDGNSFIDLGSGIAVTSVGASDPAVVAAVQEAAAHFTHTCFMVTPYEGYVAVTEQLNRLTPGDHAKRTVLFNSGAEAVENAVKVARLATGRDAVVAFDHAYHGRTNLTMALTAKAMPYKTNFGPFAPEVYRMPMSYPFREENPEITGAEAAKRAITMIEKQIGGDQVAAIIIEPIQGEGGFIVPAEGFLPALSEWAKEKGIVFIADEVQSGFCRTGEWFAVDHEGVVPDIITMAKGIAGGLPLSAITGRADLLDAVHPGGLGGTYGGNPVACAAALAAIDTMEQHDLNGRARHIEELALGKLRELAAELSAGGGSVVGDIRGRGAMLAIELVQPGSKEPNAELTKAVAAACLKEGVIILTCGTYGNVIRLLPPLVISDELLIDGLEVLAAAIKAHA

SEQ ID NO: 32 - protein, 424 aa, synthetic construct
MSKTNASLMKRREAAVPRGVGQIHPIFAESAKNATVTDVEGREFIDFAGGIAVLNTGHVHPKIIAAVTEQLNKLTHTCFQVLAYEPYVEVCEKINAKVPGDFAKKTLLVTTGSEAVENSIKIARAATGRAGVIAFTGAYHGRTMMTLGLTGKVVPYSAGMGLMPGGIFRALYPNELHGVSIDDSIASIERIFKNDAEPRDIAAIIIEPVQGEGGFYVAPKEFMKRLRALCDQHGILLIADEVQTGAGRTGTFFAMEQMGVAADLTTFAKSIAGGFPLAGVCGKAEYMDAIAPGGLGGTYAGSPIACAAALAVMEVFEEEHLLDRCKAVGERLVTGLKAIQAKYPVIGEVRALGAMIAVELFVDGDSHKPNAPAVAAVVAKARDKGLILLSCGTYGNVLRVLVPLTSPDEQLDKGLAIIEECFSE

SEQ ID NO: 33 - protein, 425 aa, synthetic construct
MSKTNASLMKRREAAVPRGVGQIHPIFAESAKNATVTDVEGREFIDFAGGIAVLNTGHLHPKIIAAVTEQLNKLTHTCFQVLAYEPYVELCEKINAKVPGDFAKKTLLVTTGSEAVENAVKIARAATGRAGVIAFTGAYHGRTMMTLGLTGKVVPYSAGMGLMPGGVFRALYPNELHGVSVDDSIASIERIFKNDAEPRDIAAIIIEPVQGEGGFYVAPKEFMKRLRALCDQHGILLIADEVQTGAGRTGTFFAMEQMGVSADLTTFAKSIAGGFPLAGVCGKAEYMDAIAPGGLGGTYAGSPIACAAALAVMEVFEEEHLLDRCKAVGERLVTGLKAIQSKYPVIGEVRALGAMIAVELFVDGDSHKPNAAAVASVVAKARDKGLILLSCGTYGNVLRVLVPLTSPDEQLDKGLAIIEECFSEL

SEQ ID NO: 34 - protein, 425 aa, synthetic construct
MSKTNASLMKRREAAVPRGVGQIHPIFAESAKNATVTDVEGREFIDFAGGIAVLNTGHLHPKIIAAVTAQLNKLTHTCFQVLAYEPYVELCEKINAKVPGDFAKKTLLVTTGSEAVENAVKIARAATGRAGVIAFTGAYHGRTMMTLGLTGKVVPYSAGMGLMPGGIFRALYPNELHGVSIDDSIASIERIFKNDAEPRDIAAIIIEPVQGEGGFYVAPKEFMKRLRALCDQHGILLIADEVQTGAGRTGTFFAMEQMGVAADLTTFAKSIAGGFPLAGVCGKAEYMDAIAPGGLGGTYAGSPIACAAALAVMEVFEEEHLLDRCKAVGERLVTGLKAIQAKYPVIGEVRALGAMIAVELFVDGDSHKPNAPAVAAVVAKARDKGLILLSCGTYGNVLRVLVPLTSPDEQLDKGLAIIEECFAEL

SEQ ID NO: 35 - protein, 420 aa, synthetic construct
MKNQDLNTRRSLATPRGVGVMCDFYAVRAENATLWDAEGKEYIDFAGGIAVLNTGHLHPKVKAAVTAQLDNFTHTAYQIVPYEGYISLAERINRVAPIDGLKKTAFFTTGVEAVENAVKIARSATGRSGVIAFSGSFHGRTMLGMALTGKVAPYKLSFGPMPGDIYHVPFPNATQSISVADSLKALDLLFKVDIDPKRVAAIIIEPVQGEGGFNITPPELMTALRKVCDEHGILLIADEVQTGFGRTGKLFAMEHHTVQADIITMAKSLGGGFPISGIVGRAEIMDGPAAGGLGGTYAGNPLAVAAAHAVLDVIAEEKLCERATQLGDKLRAHLEGLRGKVPGIADVRGLGSMVALELNDAAGKPDAEAVKRVQKRALDAGLILLSCGVYGNVLRFLYPLTIPDAQFARALDILSEALAA

SEQ ID NO: 36 - protein, 422 aa, synthetic construct
MMQRRVAAVPRGVGQIHPIFADHAKNSSVVDVEGREFIDFAGGIAVLNTGHLHPKIVKAVEEQLHKLTHTCFQVLAYEPYIALCEAIAKRVPGDFAKKTLLVTSGSEAVENAVKIARAATGRAGVIAFTGAYHGRTMMTLSLTGKVVPYSAGMGLMPGGVFRALAPCPLHGISEDDAIASIERVFKNDAQPRDIAAIIIEPVQGEGGFYVNSPAFMQRLRALCDEHGILLIADEVQTGAGRTGTFFACEQLGIVPDLTTFAKSVGGGFPISGVCGKAEIMDVIAPGGLGGTYAGSPIACAAALAVMEVFEEEKLLERSQAVGEKLKAGLNAIAARHKVIGDVRGLGSMVAIELFEGGDHSKPAAELVGRIVARARDKGLLLLSCGTYYNVIRFLMPLTIPDAQLERGLAIVAECFDELASEQ

SEQ ID NO: 37 - protein, 419 aa, synthetic construct
MTDRKNAAISRGVGMTTQIYAERAENAEIWDREGRRYIDFAAGIAVVNTGHRHPKVVQAVKDQLDRFTHTCHQVVPYENYVALAERLNDLVPGDFEKKTIFATTGAEAVENAVKIARFATGRSAIIAFTGAFHGRTFMGMTLTGKVQPYKAGFGAMMPDVFHLPFPNELHGVSQDDALAALDKLFKADVDPARVAAIIVEPVQGEGGFYPVPSGFMQKLRALCDDNGILLIADEVQTGFARTGRLFAMELHGVSADITTMAKGLGGGLPISAVTGRAEIMDAPNPGGLGGTYAGNPLGVAAAHAVLDIIEEEGLCDRAERLGQRLKQRLASLRDDVPEIVDIRGPGLMNAVEFNVAGSDRPNPEMTNRVREEALKRGLILLTCGVYGNVVRFLPPLTVPDAVFDEALGILEDSIRAARG

SEQ ID NO: 38 - protein, 471 aa, Saccharomyces cerevisiae
MSICEQYYPEEPTKPTVKTESIPGPESQKQLKELGEVFDTRPAYFLADYEKSLGNYITDVDGNTYLDLYAQISSIALGYNNPALIKAAQSPEMIRALVDRPALGNFPSKDLDKILKQILKSAPKGQDHVWSGLSGADANELAFKAAFIYYRAKQRGYDADFSEKENLSVMDNDAPGAPHLAVLSFKRAFHGRLFASGSTTCSKPIHKLDFPAFHWPHAEYPSYQYPLDENSDANRKEDDHCLAIVEELIKTWSIPVAALIIEPIQSEGGDNHASKYFLQKLRDITLKYNVVYIIDEVQTGVGATGKLWCHEYADIQPPVDLVTFSKKFQSAGYFFHDPKFIPNKPYRQFNTWCGEPARMIIAGAIGQEISDKKLTEQCSRVGDYLFKKLEGLQKKYPENFQNLRGKGRGTFIAWDLPTGEKRDLLLKKLKLNGCNVGGCAVHAVRLRPSLTFEEKHADIFIEALAKSVNEL

SEQ ID NO: 39 - protein, 450 aa, Clostridium viride
MLKNALPKIVTESLPGPKAKAIIDRRANAIPSAIRCVYPVVIDRGEGAMVEDVDGNHFLDWIGGVGVLNIGYSHPEVVDAVKKQTERYFHGMFNIVTHEGYVALAEKLSTLAPVLGKEKKAFFANSGAEANENAIKIAKAYTKRPNIIVFSGAFHGRTMLTMSMTSKKAYANGMGPFPDGVYRAKFPYYYRNPVGLPQEEAVNYYIESIKSVFEEASPPEHVAAIVVEPLQGEGGFIPAPIEWVKAVRQLCDQHGILLIADEVQTGFCRAGKMFASEYWQGAGASPDILTTAKSIAAGVPISAIVARNEIMDAVPAGIIGGTYCGNPLACAAALKVIEVMERDKFAQRSTEIGKIAMERFQQFKQNYPVVGDVRGLGSMIGLEFVKDPVSKEPNAPFVNALVQEAVKRGLMIENAGTYGNVIRFLAPLTITDEQLYAGLDIMEESIKALL

SEQ ID NO: 40 - protein, 441 aa, Gulosibacter molinativorax
MEFRLEQQRKIVTEIPGPKSEELLKRRQQYVSSSIGSSLPVYIGEADGAILRDVDGNQFIDFGSGIGVTTVGNANPKVVKAIQDAAAASTHLQINTAPYGSYVDLCQKLTELTPGDFPKKAALFNSGAEAVENAVKIARKYTGRDAVIVFDHAFHGRTNLTMAMTSKSMPYKDGFGPFAPEVYRAPLSYPFRDELSGPEAAARTIDVIDKQVGAKNVAAIIIEPIVGEGGFIVPAEGFLPALVEYANENGIVFVADEVQAGMSRTGKWFCSEWEGIEPDLVTSAKGIAGGMPLSAVVGRAEIMDAPHTGGIGGTYAGNPVANAAALATIASYEEDGLQEAALRIEKTIRGVLEPLVDELDVVGEVRGRGAMLAIELVTGADKTPNPELVKTVVAEAQAQGVVFLTCGTYGNVIRFLPPLVITDELLTDGLNVLVDAIRKNA

SEQ ID NO: 41 - protein, 447 aa, Kocuria rhizophila
MAEIEYRLPQKRELVTSIPGPKSQSLAERRAQTVAAGVASSLPVFADELDGGVIKDVDGNQMVDLGSGIAVTSVGASNPKVVARVQDAVAKFTHTCFMVTPYEDYVAVGEKMAQLTPGSFDKRTALFTSGSEAVENAVKIARVRTKRQAVVVFDHAYHGRTNLTMAMTAKVMPYKQGFGPFANEVYRVPMSYPFRDPEGMTGTEAAKRALTMVEKQVGAENVAAVVIEPIQGEGGFIVPAEGFLPALSSWCRENGAVFVADEVQAGIARTGAWFASEHEGVEPDLVTFAKGIAGGMPLSGVVGRAEIMDAVHPGGLGGTYGGNPVACAAALGALEAIEEWDLVARAQEIEKVIREELGSVAESSPVVGDLRGRGAMIALEFVKPGTTEPNPDAAKQIAARCLEQGVAILTCGTYGNIVRLLPPLVIDMDLLRDALGVFAAAIQEVGA

SEQ ID NO: 42 - protein, 468 aa, Arthrobacter sp.
MVELVETRTGTHQKGIPMTATAPDITYRLEQKRRVQADFPGPKSVALTERRKAVVAAGVASSVPVFVSDADGGIIHDVDGNSFIDLGSGIAVTSVGASDPAVVGAVKEAVEHFTHTCFMVTPYEGYVAVAEQLNRLTPGDHEKRTVLFNSGAEAVENAVKVARLATGRDAVVAFDHAYHGRTNLTMALTAKAMPYKTNFGPFAPEVYRMPMSYPFREENPAITGAEAAKRAITAIEKQIGGEQVAAIIIEPIQGEGGFIVPADGFLPALAAWAKEKGIVFIADEVQSGFCRTGEWFAVNHEGVVPDIITMAKGIAGGMPLSAITGRADLLDAVHPGGLGGTYGGNPVACAAALASIGSMEEYDLNARAKHIEELATGRLRELQGEVSVIGDIRGRGAMLAIELVQAGSKEPNPELTKAVAAACLKEGVIILTCGTYGNVIRLLPPLVIGDELLLDGLEVLAAAIKAHA

SEQ ID NO: 43 - protein, 426 aa, Pseudomonas sp.
MSKTNESLLQRRQAAVPRGVGQIHPVVAERAENATVWDVEGREYIDFAGGIAVLNTGHLHPKVVAAVQEQLTKLSHTCFQVLAYEPYIELAEEIAKRVPGNFPKKTLLVTSGSEAVENAVKIARAATGRAGVIAFTGAYHGRTMMTLGLTGKVVPYSAGMGLMPGGIFRALAPCELHGISEDESIASIERIFKNDAQPRDIAAIIIEPVQGEGGFYVNSKPFMQRLRALCDEHGILLIADEVQTGAGRTGTFFATEQLGIVPDLTTFAKSVGGGFPISGVVGKAEIMDAIAPGGLGGTYAGSPIACAAALAVLKVFDEEKLLERSQAVGETLKAGLREIQAKHKVIGDVRGLGSMVAIELFEGGDVNKPAAELVSKIVVRAREKGLILLSCGTYYNVIRFLMPVTIPDAQLKQGLAILAECFDELA

SEQ ID NO: 44 - protein, 426 aa, Pseudomonas putida
MSKTNESLMQRRVAAVPRGVGQIHPIFAESARNATVTDVEGREFIDFAGGIAVLNTGHVHPKIIAAVEAQLHKLTHTCFQVLAYEPYVEVCEKINALIPGDFAKKTLLVTTGSEAVENAIKIARAATGRAGVIAFTGAYHGRTMMTLGLTGKVVPYSAGMGLMPGGIFRAIYPNELHGVSIDDSIASIERIFKNDAEAKDIAAIILEPVQGEGGFYVAPKEFMKRLRALCDQHGILLIADEVQTGAGRTGTFFAMEQMGVAPDLTTFAKSIAGGFPLAGVCGKAEYMDAIAPGGLGGTYAGSPIACAAALAVMEVFEEEKLLDRSKAVGERLVTGLRKIQDKHPIIGDVRALGSMIAVEVFDKAGSHTPNPTAVAAVVAKARDKGLILLSCGTYGNVLRILVPLTSPDEQLDKGLAIIEECFAELA

SEQ ID NO: 45 - protein, 442 aa, Alcaligenes faecalis
MSKDKAMLNAFDPAAADSLPAAERALIERRQRVLGTAYRLFYDNPLHIVRGEGVWLYDAAGERYLDAYNNVASVGHSHPRVVAAIAEQAAILNTHTRYLHDGVVEYAERLVATFPSALSQAMFTCTGSEANDLALRIARSHTGASGVIVTELAYHGVTAAVAAVSPSLGKTVPLGVDVRAVSAPDTYRHDPATIGVWFAARVQEAIDDMLRHGIRPAALLVDTVFSSDGVYTDPAPFLAPAVEAIRAAGGLFIADEVQAGFARTGSCMWGFQRHGLVPDIVTMGKPMGNGHPIAGVVARPEIFERFGRDARYFNTFGGNPVSCAAALATLDVIQDEGLQANAARTGAYLRDKFREMARKHSWIGDVRGDGLFMGIELVKDQAAKTPATEETHRFVNLMREHRVLLSATGLRGNVIKLRPQLPFSLENADQLMQAADEVFAKL

SEQ ID NO: 46 - DNA, 1095 bp, Candida tropicalis
  source 1..1095: mol_type = genomic DNA
atgatcacggcgcaagcggttctgtacacccagcacggtgaaccaaaggatgttctgtttactcaatcctttgaaatcgatgatgataacctcgcacccaatgaagtgattgttaaaaccttgggctcgccggtcaatcctagcgatatcaaccagatccagggcgtttatccatctaagccggcgaaaaccaccggattcggtactacggaaccggcggccccgtgcggcaatgaaggcttattcgaagtaattaaagtaggtagtaacgtcagctctctggaagccggggattgggtgattcctagccacgtcaatttcgggacctggcgcacacatgccctggggaacgatgacgatttcattaaactgccgaatccggcgcaaagcaaagcgaacgggaagcctaatggcctgaccattaaccaaggcgcgacgatcagcgtgaaccccttgacggcatatctcatgctcactcattacgttaaattgacaccgggtaaggattggttcatccaaaatggtgggaccagcgctgttggtaagtacgcgtcgcaaatcggcaaacttttgaactttaactcaattagtgttattcgcgatcgtcctaacctggacgaagttgtggccagccttaaagaactgggcgccacccaggtaattaccgaagatcagaataatagcagagaattcggtccgaccattaaagaatggatcaaacagtcaggtggtgaagcgaaacttgcgctgaactgcgttgggggcaaatcgtcgaccgggattgcccgtaagctgaacaacaacggcctaatgctgacctacggaggtatgagtttccaaccagtgacaatccctacctcgttgtatatcttcaagaacttcacctccgctggcttttgggtgaccgaactgctaaaaaacaacaaagaactgaaaacatctaccttaaatcagattattgcatggtatgaagagggcaaactgacggatgcgaagtcaattgaaacactgtatgatggaacgaaacctttgcacgaattataccaggatggtgtggcgaacagtaaagacggcaaacagctgattacttattag

SEQ ID NO: 47 - DNA, 1095 bp, Candida tropicalis
  source 1..1095: mol_type = genomic DNA
atgatcacggcgcaagcggttctgtacacccagcacggtgaaccaaaggatgttctgtttactcaatcctttgaaatcgatgatgataacctcgcacccaatgaagtgattgttaaaaccttgggctcgccggtcaatcctagcgatatcaaccagatccagggcgtttatccatctaagccggcgaaaaccaccggattcggtactacggaaccggcggccccgtgcggcaatgaaggcttattcgaagtaattaaagtaggtagtaacgtcagctctctggaagccggggattgggtgattcctagccacgtcaatttcgggacctggcgcacacatgccctggggaacgatgacgatttcattaaactgccgaatccggcgcaaagcaaagcgaacgggaagcctaatggcctgaccattaaccaaggcgcgacgatcagcgtgaaccccttgacggcatatctcatgctcactcattacgttaaattgacaccgggtaaggattggttcatccaaaatggtgggaccagcgctgttggtaagtacgcgtcgcaaatcggcaaacttttgaactttaactcaattagtgttattcgcgatcgtcctaacctggacgaagttgtggccagccttaaagaactgggcgccacccaggtaattaccgaagatcagaataatagcagagaattcggtccgaccattaaagaatggatcaaacagtcaggtggtgaagcgaaacttgcgctgaactgcgttgggggcaaatcgtcgaccgggattgcccgtaagctgaacaacaacggcctaatgctgacctacggaggtatgagtttccaaccagtgacaatccctacctcgttgtatatcttcaagaacttcacctccgctggcttttgggtgaccgaactgctaaaaaacaacaaagaactgaaaacatctaccttaaatcagattattgcatggtatgaagagggcaaactgacggatgcgaagtcaattgaaacactgtatgatggaacgaaacctttgcacgaattataccaggatggtgtggcgaacagtaaagacggcaaacagctgattacttattaa

SEQ ID NO: 48 - DNA, 1023 bp, Drosophila melanogaster
  source 1..1023: mol_type = genomic DNA
atgatgagcgttgttgcaaaaagcctgaaatatacccagcatggtgaaccgcaagaagttctgcagctggttgaagataaactgcctgatccgaaagataatcaggtgctggttaaaattctggcagcaccgattaatccggcagatattaacaccattcagggtaaatatccggtgaaaccgaaatttccggcagttggtggtaatgaatgtgttgccgaagttatttgcgttggcgataaagtgaaaggttttgaagcaggtcagcatgttattccgctggcaagcggtttaggcacctggaccacacatgcagtttataaagaagatcagctgctgattgtgagcaaaaaagttggtctggcagaagcagcgaccagcaccgtgaatccgaccaccgcatatcgtatgctgaaagattttgttcagctgtgtccgggtgataccgttattcagaatggtgcaaatagcgcagttggtcaggccgttcatcagctgtgccgtgcatggggtattaatagcgttggtattgttcgtgatcgtccggaaattgcagaactgaaacaaatgctgcagtgtctgggtgcaaccgaagttctgaccgaagcagaaattcgtaccagcgatatctttaaaagcggcaaactgaaaaaaccgcgtctggcatttaattgtgttggtggcaaaagcgccaccgaagttagccgtcatctggataatggtggtgttctggttacctatggtggtatgagccgtgaaccggttaccgttgcaaccggtccgctgattttcaaagatattgcatttcgtggtttctggatgacccgttggagcaaagaaaactatagcagtccggaacgtagcaagatgttcaaagaaatttttgaactgatggaacagggcaaatttgtggcaccgaatcatgaaatggtgccgctggccaaattcaaagatgcagcagcagccgcactgagctttaaaggttttaccggcaaaaaatacatcctggatatgagcatttag

SEQ ID NO: 49 - DNA, 1023 bp, Drosophila melanogaster
  source 1..1023: mol_type = genomic DNA
atgatgagcgttgttgcaaaaagcctgaaatatacccagcatggtgaaccgcaagaagttctgcagctggttgaagataaactgcctgatccgaaagataatcaggtgctggttaaaattctggcagcaccgattaatccggcagatattaacaccattcagggtaaatatccggtgaaaccgaaatttccggcagttggtggtaatgaatgtgttgccgaagttatttgcgttggcgataaagtgaaaggttttgaagcaggtcagcatgttattccgctggcaagcggtttaggcacctggaccacacatgcagtttataaagaagatcagctgctgattgtgagcaaaaaagttggtctggcagaagcagcgaccagcaccgtgaatccgaccaccgcatatcgtatgctgaaagattttgttcagctgtgtccgggtgataccgttattcagaatggtgcaaatagcgcagttggtcaggccgttcatcagctgtgccgtgcatggggtattaatagcgttggtattgttcgtgatcgtccggaaattgcagaactgaaacaaatgctgcagtgtctgggtgcaaccgaagttctgaccgaagcagaaattcgtaccagcgatatctttaaaagcggcaaactgaaaaaaccgcgtctggcatttaattgtgttggtggcaaaagcgccaccgaagttagccgtcatctggataatggtggtgttctggttacctatggtggtatgagccgtgaaccggttaccgttgcaaccggtccgctgattttcaaagatattgcatttcgtggtttctggatgacccgttggagcaaagaaaactatagcagtccggaacgtagcaagatgttcaaagaaatttttgaactgatggaacagggcaaatttgtggcaccgaatcatgaaatggtgccgctggccaaattcaaagatgcagcagcagccgcactgagctttaaaggttttaccggcaaaaaatacatcctggatatgagcatttaa